(19) United States
(12) Patent Application Publication
(10) Pub. No.: US 2005/0197312 A1
(43) Pub. Date: Sep. 8, 2005

(54) Transcription factor RNA interference reagents and methods of use thereof

(76) Inventors: Kevin Fitzgerald, Lambertville, NJ (US); Donald G. Jackson, Lawrenceville, NJ (US); Qi Guo, Newtown, PA (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 11/070,767

(22) Filed: Mar. 2, 2005

Related U.S. Application Data
(60) Provisional application No. 60/549,730, filed on Mar. 3, 2004.

(51) Int. Cl. A61K 48/00; C12N 9/00; C12N 15/85

(52) U.S. Cl. 514/44; 435/455

(57) ABSTRACT

The present invention concerns methods and reagents useful in modulating transcription factor gene expression in a variety of applications, including methods of use in therapeutic, diagnostic, target validation, and genomic discovery applications. Specifically, the invention relates to small nucleic acid molecules, such as short interfering nucleic acid (siRNA), short interfering RNA (siRNA), and doublestranded RNA (dsRNA) molecules capable of mediating RNA interference (RNAi) against E2F1, NFkB, CREB-1, and/or CDC2A gene expression, useful in the treatment of cell cycle disorders, inflammatory conditions, reproductive disorders, cancers and any other condition that responds to modulation of E2F1, NFkB, CREB-1, and/or CDC2A expression and/or activity.

This application claims benefit to provisional application U.S. Ser. No. 60/549,730, filed Mar. 3, 2004; under 35 U.S.C. 119(e). The entire teachings of the referenced applications are incorporated herein by reference.

### FIELD OF THE INVENTION

The present invention concerns methods and reagents useful in modulating transcription factor gene expression in a variety of applications, including methods of use in therapeutic, diagnostic, target validation, and genomic discovery applications. Specifically, the invention relates to small nucleic acid molecules, such as short interfering nucleic acid (siNA), short interfering RNA (siRNA), and doublestranded RNA (dsRNA) molecules capable of mediating RNA interference (RNAi) against E2F1, NFkB, CREB-1, and/or CDC2A gene expression, useful in the treatment of cell cycle disorders, inflammatory conditions, reproductive disorders, cancers and any other condition that responds to modulation of E2F1, NFkB, CREB-1, and/or CDC2A expression and/or activity.

### BACKGROUND OF THE INVENTION

A wide variety of diseases result from the over-or under-expression of one or more genes. Given cells may make insufficient amounts of a protein (e.g. insulin) or too much of a protein, be it a normal protein (e.g. TNF), a mutant protein (e.g. an oncogene), or a non-host protein (e.g. HIV tat). The ultimate goal of therapeutic intervention in such diseases is a selective modulation of gene expression.

A variety of methods of transcriptional modulation in vitro have been reported including the use of anti-sense nucleic acids capable of binding nascent message, intracellular immunization with dominant negative mutants.

With the broad potential therapeutic applications, massive efforts have been extended by prominent laboratories and clinics around the world to extend these methods in vivo. To date, the transcription factor RNA interference strategy has never been successfully adopted in vivo.

Description of the roles of transcription factors may be found in Nevins, Science 258, 424-429 (1992); Dalton, EMBO J. 11, 11797 (1992); Yee et al. ibid. 6, 2061 (1987), Weintraub et al., Nature 358, 259-261 (1992), Pagano et al., Science 255, 1144-1147 (1992). Viral coat protein-liposome mediated transfection is described by Kaneda et al., Science 243, 375 (1989). Ritzenthaler et al. (1991) Biochem J. 280, 157-162; Ritzenthaler et al (1993) J. Biol Chem 268, 13625-13631; Bielinska et al., Science 16, 997-1000 (1990) and Sullenger et al., Cell 63, 601-608 (1990) describe inhibition of transcription with double stranded nucleic acids. RNAi is its potential applications have been reviewed extensively. Dave R S, Pomerantz R J. RNA interference: on the road to an alternate therapeutic strategy!, Rev Med Virol. November-December 2003;13(6):373-85; Cheng J C, Moore T B, Sakamoto K M. RNA interference and human disease. Mol Genet Metab. September-October 2003;80(1-2):121-8. Wilson J A, Richardson C D Induction of RNA interference using short interfering RNA expression vectors in cell culture and animal systems Curr Opin Mol Ther. August 2003;5(4):389-96.

RNA interference refers to the process of sequence-specific post-transcriptional gene silencing in animals mediated by short interfering RNAs (siRNAs) (Fire et al., 1998, Nature, 391, 806). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., 1999, Trends Genet., 15, 358). Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of dsRNA in cells triggers the RNAi response though a mechanism that has yet to be fully characterized. This mechanism appears to be different from the interferon response that results from dsRNA-mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNAs) (Berstein et al., 2001, Nature, 409, 363). Short interfering RNAs derived from dicer activity are typically about 21 to about 23 nucleotides in length and comprise about 19 base pair duplexes (Elbashir et al., 2001, Genes Dev., 15, 188). Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

RNAi has been studied in a variety of systems. Fire et al., 1998, Nature, 391, 806, were the first to observe RNAi in C. elegans. Wianny and Goetz, 1999, Nature Cell Biol., 2, 70, describe RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates. (Elbashir et al., 2001, EMBO J., 20, 6877) has revealed certain requirements for siRNA length, structure, chemical composition, and sequence that are essential to mediate efficient RNAi activity. These studies have shown that 21-nucleotide siRNA duplexes are most active when containing 3′-terminal dinucleotide overhangs. Furthermore, complete substitution of one or both siRNA strands with 2′-deoxy (2′-H) or 2′-O-methyl 5 nucleotides abolishes RNAi activity, whereas substitution of the 3′-terminal siRNA overhang nucleotides with 2′-deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′ end of the guide sequence (Elbashir et al., 2001, EMBO A, 20, 6877).

Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309).

Studies have shown that replacing the 3′-terminal nucleotide overhanging segments of a 21-mer siRNA duplex having two -nucleotide 3′-overhangs with deoxyribonucleotides does not have an adverse effect on RNAi activity. Replacing up to four nucleotides on each end of the siRNA with deoxyribonucleotides has been reported to be well tolerated, whereas complete substitution with deoxyribonucleotides results in no RNAi activity (Elbashir et al., 2001, EMBO J., 20, 6877). In addition, Elbashir et al., supra, also report that substitution of siRNA with 2′-O-methyl nucleotides completely abolishes RNAi activity. Li et al., International PCT Publication No. WO 00/44914, and Beach et al., International PCT Publication No. WO 01/68836 preliminarily suggest that siRNA may include modifications to either the phosphate-sugar backbone or the nucleoside to include at least one of a nitrogen or sulfur heteroatom, however, neither 25 application postulates to what extent such modifications would be tolerated in siRNA molecules, nor provides any further guidance or examples of such modified siRNA.

Kreutzer et al., Canadian Patent Application No. 2,359,180, also describe certain chemical modifications for use in dsRNA constructs in order to counteract activation of double-stranded RNA-dependent protein kinase PKR, specifically 2′-amino or 2′-O methyl nucleotides, and nucleotides containing a 2′-O or 4′-C methylene bridge. However, Kreutzer et al. similarly fails to provide examples or guidance as to what extent these modifications would be tolerated in siRNA molecules.

Parrish et al., 2000, Molecular Cell, 6, 1977-1087, tested certain chemical modifications targeting the unc-22 gene in C. elegans using long (>25 nt) siRNA transcripts. The authors describe the introduction of thiophosphate residues into these siRNA transcripts by incorporating thiophosphate nucleotide analogs with T7 and T3 5 RNA polymerase and observed that RNAs with two phosphorothioate modified bases also had substantial decreases in effectiveness as RNAi. Further, Parrish et al. reported that phosphorothioate modification of more than two residues greatly destabilized the RNAs in. vitro such that interference activities could not be assayed. The authors also tested certain modifications at the 2′-position of the nucleotide sugar in the long siRNA transcripts and found that substituting deoxynucleotides for ribonucleotides produced a substantial decrease in interference activity, especially in the case of Uridine to Thymidine and/or Cytidine to deoxy-Cytidine substitutions.

In addition, the authors tested certain base modifications, including substituting, in sense and antisense strands of the siRNA, 4-thiouracil, 5-bromouracil, 5-iodouracil, and 3-(aminoallyl) uracil for uracil, and inosine for guanosine. Whereas 4-thiouracil and 5-bromouracil substitution appeared to be tolerated, Parrish reported that inosine produced a substantial decrease in interference activity when incorporated in either strand. Parrish also reported that incorporation of 5-iodouracil and 3-(aminoallyl)uracil in the antisense strand resulted in a substantial decrease in RNAi activity as well.

The use of longer dsRNA has been described. For example, Beach et al., International PCT Publication No. WO 01/68836, describes specific methods for attenuating gene expression using endogenously-derived dsRNA. Tuschl et al., International PCT Publication No. WO 01/75164, describe a Drosophila in vitro RNAi system and the use of specific siRNA molecules for certain functional genomic and 25 certain therapeutic applications; although Tuschl, 2001, Chen. Biocherm., 2, 239-245, doubts that RNAi can be used to cure genetic diseases or viral infection due to the danger of activating interferon response. Li et al., International PCT Publication No. WO 00/44914, describe the use of specific dsRNAs for attenuating the expression of certain target genes. Zernicka-Goetz et al., International PCT Publication No. WO 01/36646, 30 describe certain methods for inhibiting the expression of particular genes in mammalian cells using certain dsRNA molecules. Fire et al., International PCT Publication No. WO 99/32619, describe particular methods for introducing certain dsRNA molecules into cells for use in inhibiting gene expression. Plaetinck et al., International PCT

Publication No. WO 00/01846, describe certain methods for identifying specific genes responsible for conferring a particular phenotype in a cell using specific dsRNA molecules. Mello et al., International PCT Publication No. WO 01/29058, describe the identification of specific genes involved in dsRNA-mediated RNAi. Deschamps Depaillette et al., International PCT Publication No. WO 99/07409, describe specific compositions consisting of particular dsRNA molecules combined with certain anti-viral agents. Waterhouse et al., International PCT Publication No. 99/53050, describe certain methods for decreasing the phenotypic expression of a nucleic acid in plant cells using certain dsRNAs. Driscoll et al., International PCT Publication No. WO 01/49844, describe specific DNA constructs for use in facilitating gene silencing in targeted organisms. Others have reported on various RNAi and gene-silencing systems. For example, Parrish et al., 2000, Molecular Cell, 6, 1977-1087, describe specific chemically-modified siRNA constructs targeting the unc-22 gene of C. elegans. Grossniklaus, International PCT Publication No.

WO 01/38551, describes certain methods for regulating polycomb gene expression in plants using certain dsRNAs. Churikov et al., International PCT Publication No. WO 01/42443, describe certain methods for modifying genetic characteristics of an organism using certain dsRNAs. Cogoni et al., International PCT Publication No. WO 01/53475, describe certain methods for isolating a Neurospora silencing gene and uses thereof. Reed et al., International PCT Publication No. WO 01/68836, describe certain methods for gene silencing in plants. Honer et al., International PCT Publication No. WO 01/70944, describe certain methods of drug screening using transgenic nematodes as Parkinson's Disease models using certain dsRNAs. Deak et al., International PCT Publication No. WO 01/72774, describe certain Drosophila-derived gene products that may be related to RNAi tin Drosophila. Arndt et al., International PCT Publication No. WO 01/92513 describe certain methods for mediating gene suppression by using factors that enhance RNAi. Tuschl et al., International PCT Publication No. WO 02/44321, describe certain synthetic siRNA constructs. Pachok et al., International PCT Publication No. WO 00/63364, and 30 Satishchandran et al., International PCT Publication No. WO 01/04313, describe certain methods and compositions for inhibiting the function of certain polynucleotide sequences using certain dsRNAs.

Echeverri et al., International PCT Publication No. WO 02/38805, describe certain C. elegans genes identified via RNAi. Kreutzer et al., International PCT Publications Nos. WO 02/055692, WO 02/055693, and EP 1144623 B1 describes certain methods for inhibiting gene expression using RNAi. Graham et al., International PCT Publications Nos. WO 99/49029 and WO 01/70949, and AU 4037501 describe certain vector expressed siRNA molecules. Fire et al., U.S. Pat. No. 6,506,559, describe certain methods for inhibiting gene expression in vitro using certain siRNA constructs that mediate RNAi.

E2F1 is a member of the E2F1 family of transcription factors. The E2F1 family plays a crucial role in the control of the cell cycle and action of tumor suppressor proteins. Specifically, E2F1 regulates S phase entry in the cell cycle. E2F1 has also been shown to be a target of the transforming proteins of small DNA tumor viruses. The E2F1 proteins contain several evolutionary conserved domains found in most members of the family. These domains include a DNA binding domain, a dimerization domain which determines interaction with the differentiation regulated transcription factor proteins (DP), a transactivation domain enriched in acidic amino acids, and a tumor suppressor protein association domain which is embedded within the transactivation domain. The E2F1 protein, in addition to E2F1-2 and E2F1-3, have been shown to have an additional cyclin binding domain. The E2F1 protein binds preferentially to retinoblastoma protein pRB in a cell-cycle dependent manner, and has been shown to mediate both cell proliferation and p53-dependent/independent apoptosis.

Aside from its role as a transcription factor, E2F1 has also been shown to be involved in a number of other activities that have both biological as well as therapeutic significance. For example, overexpression of E2F1 has been known to induce apoptosis and increase chemosensitivity in human pancreatic carcinoma cells (Elliott M J et al, Tumour Biol. 23(2):76-86 (2002)). Cell cycle, PARP cleavage and morphology support apoptosis as the cell death mechanism in response to E2F1 overexpression. Elliot et al also demonstrated that E2F1 overexpression, in combination with roscovitine, a chemotherapeutic agent, resulted in synergistic initiation of apoptosis and growth inhibition in pancreatic carcinoma cells. According to Elliot et al, the results indicated that E2F1 therapy may provide a potentially useful therapeutic strategy for advanced pancreatic cancer.

The role of E2F1 in mediating apoptosis in carcinoma cells and tissues appears to represent a general mechanism since this role has been corroborated by several groups in various different tissues. For example, Kuhn H et al (Eur Respir J. 20(3):703-9 (2002)) have demonstrated that adenovirus-mediated E2F1 gene transfer in nonsmall-cell lung cancer induces cell growth arrest and apoptosis and likely represents an effective treatment for nonsmall-cell lung cancer. Elliott M J et al (Clin Cancer Res. 7(11):3590-7 (2001)) have demonstrated that E2F1 overexpression in two colon cancer cell lines resulted in a greater than 25-fold reduction in cell growth and greater than 90% loss of cell viability in both cell lines. Elliot et al state that E2F1 is a potentially active gene therapy agent for the treatment of colon cancer. Calbo J et al (Cancer Gene Ther. 8(10):740-50 (2001)) demonstrated that Adenovirus-mediated wt-p16 reintroduction into p16 deficient cell lines induced cell cycle arrest or apoptosis in pancreatic cancer. The pro-apoptotic effect observed by reintroduction of p16 was directly related to the E2F1 pathway. Based upon the results, Calbo et al believe that p16 replacement therapy represents a promising pancreatic cancer treatment. Dong Y B et al (Cancer. 86(10):2021-33 (1999)) demonstrate that, Adenovirus-mediated E2F1 gene transfer efficiently induces apoptosis in melanoma cells and may be effective in the treatment of melanoma. Atienza C Jr et al (Int J Mol Med. 6(1):55-63. (2000)) demonstrated that Adenovirus-mediated E2F1 gene transfer induces an apoptotic response in human gastric carcinoma cells and is likely effective for the treatment of human gastric cancer. Yang H L et al (Clin Cancer Res. 5(8):2242-50 (1999)) also demonstrate that Adenovirus-mediated E2F1 gene transfer inhibits MDM2 expression and efficiently induces apoptosis in MDM2-overexpressing tumor cells. MDM2 is an oncoprotein that binds and inactivates p53. Since MDM2-overexpressing tumors are often resistant to p53 gene therapy, adenovirus-mediated E2F1 gene therapy may be a promising alternative strategy. Moreover, Stevens C et al have demonstrated that checkpoint kinase 2 (chk2) phosphorylates and activates E2F1 in response to DNA damage, resulting in apoptosis and suggests a role for E2F1 in checkpoint control and tumour suppression (Nat Cell Biol. 5(5):401-9 (2003)).

Cell cycle division 2 kinase protein (CDK2) is a member of the Ser/Thr protein kinase family, and is a catalytic subunit of the highly conserved protein kinase complex known as M-phase promoting factor (MPF), which is essential for G1/S and G2/M phase transitions of the eukaryotic cell cycle. Mitotic cyclins stably associate with this protein and function as regulatory subunits. The kinase activity of this protein is controlled by cyclin accumulation and destruction through the cell cycle. The phosphorylation and dephosphorylation of this protein also plays important regulatory roles in cell cycle control.

Like E2F1, CDK-2 is also involved in a number of other activities that have both biological as well as therapeutic significance. For example, CDK-2 was shown to be involved in the genesis or progression of malignant lymphoma and cdc2 and to represent a useful marker for response to chemotherapy based upon the significant correlative expression of CDK-2 in various clinico-pathologic disease phenotypes (Jin Y H et al., J Korean Med Sci. 17(3):322-7 (2002)). Sui L et al determined that CDK-2 is directly implicated in the incidence of malignancy in epithelial ovarian tumors (Gynecol Oncol., 83(l):56-63 (2001)). Moreover, Sui et al were also able to demonstrate that CDK-2 expression alone or in combination with p27 and cyclin-E expression could be useful in diagnosing epithelial ovarian tumors. In particular, patients with the p27 (−)/cyclin E (++)/cdk2 (++)phenotype were significantly associated with the poorest overall survival. Sui et al state that the combined evaluation of p27/cyclin E/cdk2 may provide the most important prognostic implication in diagnosing epithelial ovarian tumors.

CDK-2 has also been implicated in Alzheimer's and other neurological disorders. Johansson A et al provide evidence demonstrating a direct association for a high frequency polymorphism in the CDK-2 gene (Ex6+7I) in patients with Alzheimer's disease and fronto-temporal dementia (Neurosci Lett., 340(l):69-73 (2003)). Milton provides additional evidence supporting this link by demonstrating that CDK-2 phosphorylates amyloid-beta at the serine 26 (Neuroreport. 4;12(17):3839-44 (2001)). Phosphorylated Abeta (pSAbeta) was found in extracts from NT-2 neurons and AD brain. In NT-2 neurons the levels of pSAbeta were increased in the presence of exogenous Abeta and this increase was prevented by a cdc2 protein kinase inhibitor, olomoucine, that also prevented Abeta cytotoxicity suggesting that Abeta phosphorylation by cdc2 could play a role in the brain pathology of AD.

NFKB is a transcription regulator that is activated by various intra- and extra-cellular stimuli including cytokines, oxidant-free radicals, ultraviolet irradiation, and bacterial or viral products. NFkB plays a critical role in plastic transformation and malignant progression of various cell types. Activated NFKB translocates into the nucleus and stimulates the expression of genes involved in a wide variety of biological functions. Inappropriate activation of NFKB has been associated with a number of inflammatory diseases while persistent inhibition of NFKB has been shown to lead to inappropriate immune cell development and/or delayed cell growth.

Like E2F1 and CDK-2, NFkB is also involved in a number of other activities that have both biological as well as therapeutic significance. For example, NFKB has been shown to have utility in the treatment of tumors. Tsai P W et al demonstrate that up-regulation of vascular endothelial growth factor C, a critical activator of tumor lymphangiogenesis that has been strongly implicated in the tumor metastasis process, in breast cancer cells by heregulin-beta 1 suggesting a critical role of p38/nuclear factor-kappa B signaling pathway. This finding is significant since NF-kappa B has been shown to be involved in interleukin-1 beta- or tumor necrosis factor-alpha-induced VEGF-C mRNA expression in human fibroblasts. Tsia provided data demonstrating that HRG-beta 1 could stimulate NF-kappa B nuclear translocation and DNA-binding activity via the I kappa B alpha phosphorylation-degradation mechanism. Moreover, blockage of the NF-kappa B activation cascade caused a complete inhibition of the HRG-beta 1-induced elevation of VEGF-C.

Fujioka S et al demonstrate that NFkappaB signaling can suppress the angiogenic potential and metastasis of pancreatic cancer (Clin Cancer Res. 9(l):346-54 (2003)). NFkappaB is known to be activated constitutively in human pancreatic adenocarcinoma and human pancreatic cancer cell lines but not in normal pancreatic tissues or in immortalized/non-tumorigenic pancreatic epithelial cells, suggesting that NFkappaB plays a critical role in development of pancreatic adenocarcinoma. Inhibition of NFkappaB signaling can suppress the angiogenic potential and metastasis of pancreatic cancer, and suggests that the NFkappaB signaling pathway is a potential target for anticancer agents.

Kim J M et al demonstrate that NF-kappaB can be a central regulator of chemokine gene expression in Bacteroides fragilis enterotoxin-stimulated intestinal epithelial cells and may be an important regulator of neutrophil migration (Clin Exp Immunol. 130(l):59-66 (2002)).

Andela V B et al show that NfkappaB is involved in bone biology as well by demonstrating that it is capable of negatively regulating osteoblast differentiation and is implicated in the pathogenesis and progression of osteosarcomas (Biochem Biophys Res Commun., 297(2):237-41(2002)). The results showed that inhibition of NFkappaB signaling activity in Saos-2 cells resulted in a marked decrease in cellular proliferation, assessed by the incorporation of radioactive thymidine into cellular DNA. Decreased cellular proliferation was accompanied by the induction of bone morphogenic proteins (BMP) 4, 7, and the osteoblast specific transcription factor, Cbfa1, heralding osteoblast differentiation.

NFkB has also been associated with the incidence of rheumatoid arthritis.

Muller-Ladner U et al provide a review on the role of NFkB in the incidence of RA (Curr Rheumatol Rep. 4(3):201-7 (2002)). Gene transcription factors such as nuclear factor kB (NFkB) are activated by extracellular signals or cell-to-cell interactions that are converted into intracellular activation signals through receptor molecules located in the cell membrane. The number of known genes being translated after NFkB activation is increasing steadily. These genes includes cytokines, chemokines, growth factors, cellular ligands, and adhesion molecules. Because many of these genes are part of the pathogenesis of RA, there is considerable interest in the evaluation of the synovium-specific effects of NFkB to unveil its potential for future therapeutic strategies. The goal is to evolve these strategies from the therapies that have a wide spectrum of effects and side effects into rheumatoid arthritis-specific therapies designed to inhibit distinct molecular pathways within the synovium.

Moreover, inappropriate activation of NF-kappaB has been linked to inflammatory events associated with autoimmune arthritis, asthma, septic shock, lung fibrosis, glomerulonephritis, atherosclerosis, and AIDS. In contrast, complete and persistent inhibition of NF-kappaB has been linked directly to apoptosis, inappropriate immune cell development, and delayed cell growth. Therefore, development of modulatory strategies targeting this transcription factor may provide a novel therapeutic tool for the treatment or prevention of various diseases (Clin Chem. 45(1):7-17 (1999)).

Most human diseases can be ascribed to the aberrant activation and expression of genes whose products are involved in the initiation and progression of pathogenesis. Such diseases include autoimmune arthritis, glomerulonephritis, asthma, inflammatory bowel disease, septic shock, lung fibrosis, carcinogenesis, and AIDS. In general, these genes are quiescent or have minimal activity in affecting biological and physiological processes. However, under certain conditions that include exposure to environmental pollutants, these genes are abruptly turned on by a preexisting genetic switch, causing their overexpression. Part of this genetic switch is controlled by nuclear factor-B (NF-B), 1 an essential transcription factor that controls the gene expression of cytokines, chemokines, growth factors, and cell adhesion molecules as well as some acute phase proteins.

Additional information on NFkB may be found by reference to the following publications which are hereby incorporated by reference in their entirety herein: [1] Baldwin A S, Jr. Annu Rev Immunol, 14:649-683 (1996); [2] Finco T S, Baldwin A S, Immunity, 3:263-272 (1995); and [3] Barnes P J, Karin M, New Engl J Med, 366:1066-1071 (1997).

cAMP-response element binding protein (CREB1) is a member of the bZIP transcription factor family, which is commonly referred to as the leucine zipper family of DNA binding proteins. This protein binds as a homodimer to the cAMP-responsive element (CRE), an octameric palindrome, in the promoter of target genes. CREB1 is phosphorylated by several protein kinases, and induces transcription of genes in response to hormonal stimulation of the cAMP pathway., both are activated by protein kinase A (PKA) phosphorylation that enables binding of CREB binding protein (CBP)

CREB1 has been shown to associate with TIF2 and found to be essential for cellular transformation as demonstrated by Deguchi K et al (Cancer Cell. 3(3):259-71 (2003)). Specifically, Deguchi et al have shown that MOZ-TIF2-induced acute myeloid leukemia requires the C2HC nucleosome recognition motif of MOZ and TIF2-mediated recruitment of CREB binding protein (CBP). The results indicated that nucleosomal targeting by MOZ and recruitment of CBP by TIF2 are critical requirements for MOZ-TIF2 transformation.

Accumulating data during recent years has established that the cAMP-dependent signal transduction pathway is a major regulatory mechanism that operates at different stages of spermatogenesis. For example, in Sertoli cells, CREB levels fluctuate in a cyclical manner that depends on the specific cell associations along the spermatogenic wave. Follicle stimulating hormone (FSH) activates the cAMP signaling pathway and consequently, CREB positively auto-regulates its own expression (by binding to a CRE like element in its promoter). Subsequently, activated CREB activates transcription of genes essential for proper germ cell differentiation. In addition, TNF-alpha secreted by round spermatids, activates NF-kappaB dependent CREB expression in Sertoli cells and thus, contributes to the elevated CREB levels as long as these cells are intimately associated. Inducible cAMP early repressor (ICER) also activated by CREB, down regulates CREB expression together with its own expression, resetting CREB to basal level that enables a new spermatogenic wave. These and other findings suggest that the expanding CREB proteins and potentially other members of the CREB family are key molecular regulators at all stages of spermatogenesis (Mol Cell Endocrinol. 22; 187(1-2):115-24 (2002)).

CREP has also been associated with antidepression, neuropathy diseases, and cognition. Lai I C et al demonstrate that phosphorylated cAMP response element-binding protein (CREB), a downstream target of the cAMP signaling pathway, has been reported to be a molecular state marker for the response to antidepressant treatment in patients with major depressive disorder (MDD). In this role, CREB has been attributed to the inherent lag observed with antidepressant therapy—such a lag is thought to be due to neural plasticity, which may be mediated by the coupling of receptors to their respective intracellular signal transduction pathways. Therefore, in order to explore the role of CREB expression in MDD, quantitative reverse transcriptase-polymerase chain reaction was used to quantify CREB messenger RNA of the peripheral lymphocytes obtained from 21 MDD patients, before and after antidepressant treatment, and in 21 normal controls The results revealed no significant difference of CREB expression between untreated MDD patients and normal controls. However, after 8 weeks of antidepressant treatment, CREB expression was significantly decreased in MDD patients (p=0.025) Importantly, the CREB change was not associated with the types of antidepressants administered and therapeutic response (Neuropsychobiology, 48(4): 182-5 (2003)).

Sugars K L et al have shown that decreased CRE-mediated transcription represents an early event in exon 1 and full-length cell models of Huntington's disease that contributes to polyglutamine pathogenesis. Specifically, three CREB-binding protein-dependent transcriptional pathways, regulated by CRE, RARE and NF-kB, show abnormalities in a stable inducible PC12 cell lines expressing wild-type or mutant forms of huntingtin exon 1 fragments or the full-length huntingtin protein our exon 1 cell model. Huntingtons disease (HD) is one of nine neurodegenerative diseases caused by an expanded polyglutamine (polyQ) tract within the disease protein. In light of these results, reduced CRE-dependent transcription may contribute to polyQ disease pathogenesis since overexpression of transcriptionally active CREB, but not an inactive form of the protein, is able to protect against polyQ-induced cell death and reduce aggregation.

Marambaud P et al show that A CBP binding transcriptional repressor produced by the PS1/epsilon-cleavage of N-cadherin is inhibited by PS1 FAD mutations. Presenilin1 (PS1), a protein implicated in Alzheimer's disease (AD), forms complexes with N-cadherin, a transmembrane protein with important neuronal and synaptic functions. The results indicate that a PS1-dependent gamma-secretase protease activity promotes an epsilon-like cleavage of N-cadherin to produce its intracellular domain peptide, N-Cad/CTF2. PS1 mutations associated with familial AD (FAD) and a gamma-secretase dominant-negative mutation inhibit N-Cad/CTF2 production and upregulate CREB-mediated transcription indicating that FAD mutations cause a gain of transcriptional function by inhibiting production of transcriptional repressor N-Cad/CTF2. These data suggest that FAD mutation-induced transcriptional abnormalities maybe causally related (Cell. 114(5):635-45 (2003)).

CREB has also been attributed with having a memory enhancer function (see Tully T et al, Nat Rev Drug Discov. 2(4):267-77 (2003)).

Aside from the role of CREB as a transcription factor per se, or its involvement in neurological processes and/or diseases, significant evidence has been obtained to link CREB to GPCR regulation as well. The majority of neuropeptides have been shown to signal via G-protein-coupled receptors (GPCRs, reviewed in Bockaert & Pin, EMBO J., 18, 1723 1729 (1999)). The GPCRs are a super family of receptors typified by seven alpha helical transmembrane spanning domains linked by intra- and extracellular loops with extracellular N-terminal and intracellular C-terminal domains. Neuropeptides (and hormones, growth factors and cytokines) mediate their affects through one subgroup of GPCRs which are characterized by the presence of particular conserved amino acids (Probst et al., DNA Cell Biol., 11, 120 (1992)); Bockaert & Pin, EMBO J., 18, 1723 1729 (1999)).

There is emerging evidence that some of these receptors are regulated by intracellular levels of c-AMP and encode c-AMP response elements (CRE) in their promoter regions (reviewed in Min et al., Proc. Natl Acad. Sci. USA, 91, 9081 9085 (1994); Minowa et al. 1996; Montminy, DNA Cell Biol., 15:759 767 (1997); Herdegen & Leah, Brain Res. Reviews, 28:379 490 (1998)).

A strategy to study GPCR was to isolate of a family of highly homologous receptor genes that are differentiated from each other by differences in their promoter regions. Interestingly, all promoters encode multimeric CREs which could theoretically bind CREB protein (Montminy, Annu. Rev. Biochem., 66:807 822 (1997)). In situ hybridization experiments have mapped the pattern of expression of the Lymnaea receptors to neurons in the CNS known to be involved in ion and water metabolism (de Witt et al., Peptides, 14:783 0789 (1993); Li et al., J. Biol. Chem., 269:30288 30292 (1994)); feeding (Yeoman et al., J. Neurophysiol., 72:1357 1363 (1994)), and learning and memory (Korneev et al., J. Neurosci., 35, 65 76 (1998)).

CREB has also been associated with chronic pain. Hoeger-Bement and Sluka demonstrate that phosphorylation of CREB and mechanical hyperalgesia is reversed by blockade of the cAMP pathway in a time-dependent manner after repeated intramuscular acid injection (J Neurosci., 23(13):5437-45 (2003)). Spinal activation of the cAMP pathway produces mechanical hyperalgesia, sensitizes nociceptive spinal neurons, and phosphorylates the transcription factor cAMP-responsive element binding protein (CREB), which initiates gene transcription. Activation of the cAMP pathway in the spinal cord phosphorylates CREB and produces mechanical hyperalgesia associated with intramuscular acid injections. The mechanical hyperalgesia and phosphorylation of CREB depend on early activation of the cAMP pathway during the first 24 hr but are independent of the cAMP pathway by 1 week after intramuscular injection of acid.

The present invention provides, for the first time, validated siRNA reagents that are useful for decreasing the level of expression and/or activity of E2F1, NFKB, CREB-1, and CDC2A transcription factors. Using the above examples, it is clear the availability of novel siRNA reagents specific for the E2F1, NFkB, CREB-1, and CDC2A transcription factors provides an opportunity for therapeutic intervention for any disorder known to be associated with E2F1, NFkB, CREB-1, or CDC2A. Moreover, such siRNA reagents may also be useful in screens to identify agonists of E2F1, NFkB, CREB-1 or CDC2A.

### BRIEF SUMMARY OF THE INVENTION

The invention provides for the therapeutic treatment of diseases associated with the binding of endogenous transcription factors to genes involved in cell growth, differentiation and signaling or to viral genes.

The invention also relates to a nucleic acid from about 8 to about 30 nucleotides in length, preferably from about 15 to about 25 nucleotides in length, more preferably from about 19 to about 23 nucleotides in length, that specifically hybridizes to a nucleic acid molecule encoding the a E2F1 polypeptide, wherein said nucleic acid inhibits the expression and/or activity of the E2F1 polypeptide. Preferred nucleic acids for targeting the coding sequence of the E2F1 polypeptide are selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and/or SEQ ID NO:12.

The invention further relates to a method of inhibiting the expression of the a E2F1 polypeptide of the present invention in human cells or tissues comprising contacting said cells or tissues in vitro, in vivo, or ex vivo with a nucleic acid of the present invention so that expression of the E2F1 polypeptide is inhibited.

The present invention is also directed to a method of identifying a compound that modulates the biological activity of E2F1, the E2F1 pathway, and/or E2F1-regulated downstream effectors, comprising the steps of, (a) combining a candidate modulator compound with E2F1 in the presence of a nucleic acid that antagonizes the expression and/or activity of the E2F1 polypeptide selected from the group consisting of SEQ ID NO:1, 2, 3, 4, 5, 6, 7, 8, 9, 10, 11, and/or 12, and (b) identifying candidate compounds that reverse the antagonizing effect of the nucleic acid.

The invention also relates to a nucleic acid 8 to 30 nucleotides in length that specifically hybridizes to a nucleic acid molecule encoding the a NFkB polypeptide, wherein said nucleic acid inhibits the expression and/or activity of the NFkB polypeptide. Preferred nucleic acids for targeting the coding sequence of the NFkB polypeptide are selected from the group consisting of: SEQ ID NO:13, SEQ ID NO:14, SEQ ID NO:15, SEQ ID NO:16, SEQ ID NO:17, SEQ ID NO:18, SEQ ID NO:19, and/or SEQ ID NO:20.

The invention further relates to a method of inhibiting the expression of the a NFkB polypeptide of the present invention in human cells or tissues comprising contacting said cells or tissues in vitro, in vivo, or ex vivo with a nucleic acid of the present invention so that expression of the NFkB polypeptide is inhibited.

The present invention is also directed to a method of identifying a compound that modulates the biological activity of NFkB, the NFkB pathway, and/or NFkB-regulated downstream effectors, comprising the steps of, (a) combining a candidate modulator compound with NFkB in the presence of a nucleic acid that antagonizes the expression and/or activity of the NFkB polypeptide selected from the group consisting of SEQ ID NO:13, 14, 15, 16, 17, 18, 19, and/or 20, and (b) identifying candidate compounds that reverse the antagonizing effect of the nucleic acid.

The invention also relates to a nucleic acid 8 to 30 nucleotides in length that specifically hybridizes to a nucleic acid molecule encoding the a CREB-1 polypeptide, wherein said nucleic acid inhibits the expression and/or activity of the CREB-1 polypeptide. Preferred nucleic acids for targeting the coding sequence of the CREB-1 polypeptide are selected from the group consisting of: SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, and/or SEQ ID NO:32.

The invention further relates to a method of inhibiting the expression of the a CREB-1 polypeptide of the present invention in cells or tissues comprising contacting said cells or tissues in vitro, in vivo, or ex vivo with a nucleic acid of the present invention so that expression of the CREB-1 polypeptide is inhibited.

The present invention is also directed to a method of identifying a compound that modulates the biological activity of CREB-1, the CREB-1 pathway, and/or CREB-1-regulated downstream effectors, comprising the steps of, (a) combining a candidate modulator compound with CREB-1 in the presence of a nucleic acid that antagonizes the expression and/or activity of the CREB-1 polypeptide selected from the group consisting of SEQ ID NO:21, 22, 23, 24, 25, 26, 27, 28, 29, 30, 31, 32, 33, 34, and/or 35, and (b) identifying candidate compounds that reverse the antagonizing effect of the nucleic acid.

The invention also relates to a nucleic acid 8 to 30 nucleotides in length that specifically hybridizes to a nucleic acid molecule encoding the a CDC2A polypeptide, wherein said nucleic acid inhibits the expression and/or activity of the CDC2A polypeptide. Preferred nucleic acids for targeting the coding sequence of the CDC2A polypeptide are selected from the group consisting of: SEQ ID NO:33, SEQ ID NO:34, and/or SEQ ID NO:35.

The invention further relates to a method of inhibiting the expression of the a CDC2A polypeptide in cells or tissues comprising contacting said cells or tissues in vitro, in vivo, or ex vivo with a nucleic acid of the present invention so that expression of the CDC2A polypeptide is inhibited.

The present invention is also directed to a method of identifying a compound that modulates the biological activity of CDC2A, the CDC2A pathway, and/or CDC2A-regulated downstream effectors, comprising the steps of, (a) combining a candidate modulator compound with CDC2A in the presence of a nucleic acid that antagonizes the expression and/or activity of the CDC2A polypeptide selected from the group consisting of SEQ ID NO:33, 34, and/or 35, and (b) identifying candidate compounds that reverse the antagonizing effect of the nucleic acid.

The present invention is also directed to a method of inhibiting one or more transcription factors, transcription factor pathways, and/or transcription factor-regulated downstream effectors in cells or tissues, comprising the step of contacting said cells or tissues in vitro, in vivo, or ex vivo with one or more of the nucleic acids of the present invention, or any combination thereof, under conditions in which the expression of said transcription factor, the activity of said transcription factor pathway, and/or the activity of said transcription factor-regulated downstream effector in said cells or tissues is inhibited.

The present invention is also directed to a method of inhibiting one or more transcription factors, transcription factor pathways, and/or transcription factor-regulated downstream effectors in cells or tissues, comprising the step of contacting said cells or tissues in vitro, in vivo, or ex vivo with one or more of the nucleic acids of the present invention, or any combination thereof, under conditions in which the expression of said transcription factor, the activity of said transcription factor pathway, and/or the activity of said transcription factor-regulated downstream effector in said cells or tissues is inhibited; wherein said nucleic acids of the present invention, or said any combination thereof, is further combined with a small molecule compound, an antibody, or any other modulator of said transcription factor, prior to, subsequent, or in conjunction with contact of said cells or tissues with said nucleic acids of the present invention, or said any combination thereof.

In one embodiment, a composition of the present invention is a double-stranded nucleic acid.

In one embodiment, a composition of the present invention is double-stranded nucleic acid comprised of RNA.

In one embodiment, a composition of the present invention is a double-stranded nucleic acid comprised of DNA.

In one embodiment, a composition of the present invention is a double-stranded nucleic acid comprised of a combination of DNA and RNA.

In one embodiment, a composition of the present invention is an RNAi reagent.

In one embodiment, a composition of the present invention is an RNAi reagent in a form capable of entering target cells of a sample.

Methods and compositions are provided for blocking the capacity of endogenous trans-activating factors to modulate gene expression and thereby regulating pathological processes including inflammation, intimal hyperplasia, angiogenesis, neoplasia, immune responses, neurological disorders, and viral infections.

In one embodiment, the present invention encompasses methods of administering in vivo a composition capable of targeting an endogenous transcription factor and specifically binding to the transcript of said transcription factor.

In another embodiment, the present invention encompasses methods of administering in vivo a composition capable of targeting an endogenous transcription factor and specifically binding to the transcript of said transcription factor, wherein said binding results in the degradation of said transcript of said transcription factor.

In yet another embodiment, the present invention encompasses methods of administering in vivo a composition capable of targeting an endogenous transcription factor and specifically binding to the transcript of said transcription factor, wherein said binding results in the degradation of said transcript of said transcription factor, and further wherein said degradation results in either a decreased level of transcription factor activity or a decreased level of transcription factor protein, or both.

In one embodiment, the present invention encompasses methods of administering ex vivo a composition capable of targeting an endogenous transcription factor and specifically binding to the transcript of said transcription factor.

In another embodiment, the present invention encompasses methods of administering ex vivo a composition capable of targeting an endogenous transcription factor and specifically binding to the transcript of said transcription factor, wherein said binding results in the degradation of said transcript of said transcription factor.

In yet another embodiment, the present invention encompasses methods of administering ex vivo a composition capable of targeting an endogenous transcription factor and specifically binding to the transcript of said transcription factor, wherein said binding results in the degradation of said transcript of said transcription factor, and further wherein said degradation results in either a decreased level of transcription factor activity or a decreased level of transcription factor protein, or both.

In one embodiment, the present invention encompasses methods of administering in vitro a composition capable of targeting an endogenous transcription factor and specifically binding to the transcript of said transcription factor.

In another embodiment, the present invention encompasses methods of administering in vitro a composition capable of targeting an endogenous transcription factor and specifically binding to the transcript of said transcription factor, wherein said binding results in the degradation of said transcript of said transcription factor.

In yet another embodiment, the present invention encompasses methods of administering in vitro a composition capable of targeting an endogenous transcription factor and specifically binding to the transcript of said transcription factor, wherein said binding results in the degradation of said transcript of said transcription factor, and further wherein said degradation results in either a decreased level of transcription factor activity or a decreased level of transcription factor protein, or both.

It is another object of this invention to provide methods of pressurized intracellular delivery of compositions that do not cause distension and trauma in the target cells or tissue.

It is yet another object of this invention to allow high-efficiency intracellular delivery of naked compositions (e.g., nucleic acids, nucleic acids free of delivery vehicles, etc).

It is yet another object to allow intracellular delivery of nucleic acids under controlled incubation pressures applied for controlled incubation periods, wherein said administration of said composition is delivered under pressure.

In one embodiment, the present invention encompasses methods of administering ex vivo a composition capable of targeting an endogenous transcription factor and specifically binding to the transcript of said transcription factor, wherein said administration of said composition is delivered under pressure.

In another embodiment, the present invention encompasses methods of administering ex vivo a composition capable of targeting an endogenous transcription factor and specifically binding to the transcript of said transcription factor, wherein said binding results in the degradation of said transcript of said transcription factor.

In yet another embodiment, the present invention encompasses methods of administering ex vivo a composition capable of targeting an endogenous transcription factor and specifically binding to the transcript of said transcription factor, wherein said binding results in the degradation of said transcript of said transcription factor, and further wherein said degradation results in either a decreased level of transcription factor activity or a decreased level of transcription factor protein, or both, wherein said administration of said composition is delivered under pressure.

In yet another embodiment, the compositions of the invention are preferably administered in any of the aforementioned methods under conditions in which binding of target endogenous transcription factor to its cognate binding site is effectively inhibited.

In yet another embodiment, the compositions of the invention are preferably administered according to any of the aforementioned methods under conditions in which binding of the target endogenous transcription factor to its cognate binding site is effectively inhibited, either directly or indirectly, preferably said binding is inhibited without significant toxicity to the cells or tissues.

In another embodiment, binding of the compositions of the invention to the target endogenous transcription factor transcript results in up-regulation of genes under the control of said target endogenous transcription factor.

In another embodiment, binding of the compositions of the invention to the target endogenous transcription factor transcript results in the down-regulation of genes under the control of said target endogenous transcription factor.

In another embodiment, binding of the compositions of the invention to the target endogenous transcription factor transcript results in the up-regulation of some genes and the down-regulation of some genes under the control of said target endogenous transcription factor.

Preferably, compositions of the present invention have pharmacokinetics sufficient for effective therapeutic use in any of the aforementioned methods.

The invention further relates to a method for preventing, treating, or ameliorating a medical condition with the RNAi reagent provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, and SEQ ID NO:35, wherein the medical condition is a member of the group consisting of: inflammatory disorders, intimal hyperplasia, angiogenesis, neoplasia, immune disorders, neurological disorders, viral infections, disorders associated with E2F1, disorders associated with aberrant E2F1 activity and/or expression, cell cycle disorders, cell cycle disorders associated with aberrant function of the S-phase check point, cell cycle disorders associated with aberrant function of the G2/S-phase check point, disorders associated with p53-dependent apoptosis, disorders associated with p53-independent apoptosis, cell cycle disorders associated with aberrant cyclin D1 regulation and/or function, cell cycle disorders associated with aberrant CDC2A regulation and/or function, cell cycle disorders associated with aberrant caspase-3 regulation and/or function, proliferative disorders, proliferative disorders of the pancreas, human pancreatic carcinoma, proliferative disorders of the lung, nonsmall-cell lung cancer, proliferative disorders of the colon, colon cancer, proliferative disorders of the skin, skin cancer, proliferative disorders of the stomach, proliferative disorders of the gastrointestinal system, gastric cancer, MDM2-dependent proliferative disorders, checkpoint kinase 2 related disorders, G1 cell cycle checkpoint disorders, G2 cell cycle checkpoint disorders, aberrant cell cycle checkpoint protein disorders, disorders associated with aberrant CDK2 protein expression and/or activity, proliferative disorders of the immune system, proliferative disorders of leukemic cells, malignant lymphoma, proliferative disorders of the ovary, epithelial ovarian tumors, neural disorders, neurodegenerative disorders, Alzheimers, disorders associated with aberrant amyloid-beta expression and/or activity, disorders associated with aberrant NFKB expression and/or activity, disorders associated with aberrant cytokine expression and/or activity, disorders associated with high levels of oxidant-free radicals, disorders associated with high levels of ultraviolet irradiation, inflammatory disorders, rheumatoid arthritis, aberrant immune cell development, aberrant immune cell growth, disorders associated with aberrant vascular endothelial growth factor C expression and/or activity, disorders associated with tumor lymphangiogenesis, tumor metastasis process, proliferative disorder of the breast, breast cancer, disorders associated with aberrant heregulin-beta 1 expression and/or activity, disorders associated with interleukin-1 beta activity and/or expression, disorders associated with aberrant angiogenic potential of tissues, tumors, pancreatic adenocarcinoma, disorders associated with aberrant neutrophil migration, bone disorders, disorders associated with aberrant osteoblast differentiation, proliferative disorders of bone cells and tissues, osteosarcomas, disorders associated with aberrant expression and/or activity of bone morphogenic proteins (BMP) 4, disorders associated with aberrant expression and/or activity of BMP7, disorders associated with aberrant expression and/or activity of Cbfa1, disorders associated with aberrant osteoblast differentiation, autoimmune disorders, arthritis, asthma, septic shock, lung fibrosis, glomerulonephritis, atherosclerosis, AIDS, aberrant apoptosis, inappropriate immune cell development, delayed cell growth, disorders associated with aberrant expression and/or activity of cAMP-response element binding protein (CREB1), acute myeloid leukemia, reproductive disorders, spermatogenesis, major depressive disorder, neuropathies, Huntington's disease, disorders associated with aberrant N-cadherin expression and/or activity, disorders associated with aberrant G-protein coupled receptor regulation and/or expression, pain disorders, chronic pain, restinosis, restinosis of vascular smooth muscle cells, disorders associated with neointima formation, proliferative lesions, and proliferative lesions in mammalian blood vessels.

The present invention is also directed to methods for treating, ameliorating, and/or preventing restenosis in a mammalian host, said method comprising introducing an E2F1-directed siRNA reagent of the present invention into vascular smooth muscle cells at the site of a vascular lesion in vitro, in vivo, or ex vivo, said cells capable of resulting in restenosis as a result of neointima formation, in an amount to inhibit said neointima formation, whereby said E2F1-directed siRNA reagent of the present invention is characterized by having a sequence specific for binding to an E2F1 transcription factor.

The present invention is also directed to methods for treating, ameliorating, and/or preventing proliferative lesion formation in a mammalian blood vessel in vitro, in vivo, or ex vivo, said method comprising introducing into vascular smooth muscle cells of said blood vessel E2F1-directed siRNA reagent of the present invention that comprises a sequence that is specific for binding to transcription factor E2F; in an amount sufficient to inhibit proliferative lesion formation in said blood vessel.

### BRIEF DESCRIPTION OF THE FIGURES/DRAWINGS

The file of this patent application contains at least one Figure executed in color. Copies of this patent with color Figure(s) will be provided by the Patent and Trademark Office upon request and payment of the necessary fee.

FIG. 1 shows transfection of 2×10⁵ HeLa cells with one of four RNAi reagents (“BMS-E2F1-1”; “BMS-E2F1-2”; “BMS-E2F1-3”; or “BMS-E2F1-4”) designed to target the E2F1 receptor transcript (Genbank Accession No. gi|NM_{—}005225), results in significant knock-down of E2F1 transcript levels. A fluorescently labeled RNAi reagent (“BMS-FITC-RNAi”) was used as a control to assess the transfection efficiency level. In addition, RNAi reagent specific to GFP B was used as a non-specific negative control (“BMS-GFP-B”). Percent knockdown was determined by measuring transcript levels in transfected versus non-transfected HeLa cells using RT-PCR as described in Example 3, generally. The experiment was performed as described in Example 4 herein.

FIG. 2 shows transfection of 2×10⁵ HeLa cells with one of four RNAi reagents (“NFkBp65-1”; “NFkBp65-2”; “NFkBp65-3”; or “NFkBp65-4”) designed to target the Homo sapiens v-rel reticuloendotheliosis viral oncogene homolog A, nuclear factor of kappa light polypeptide gene enhancer in B-cells 3, p65 (avian) (NFkB-p65; Genbank Accession No. gi|NM_{—}006509) transcript results in significant knock-down of NFkB-p65 protein levels. Untransfected HeLa cells (“Untrans”) and HeLa cells transfected with a non-specific RNAi reagent (“Mock”) were used as negative controls. Percent knockdown was determined by measuring protein levels in transfected versus non-transfected HeLa cells by quantifying the band intensity as described in Example 5.

FIG. 3 shows transfection of 2×10⁵ HeLa cells with one of four RNAi reagents (“BMS-CREB1-1”; “BMS-CREB1-2”; “BMS-CREB1-3”; or “BMS-CREB1-4”) designed to target the cAMP responsive element binding protein 1 (CREB; Genbank Accession No. gi|NM_{—}004379) transcript results in significant knock-down of CREB transcript levels. HeLa cells transfected with a RNAi reagent specific to GFP B transcript (“BMS-GFP-B”), and an RNAi reagent specific to CDC2A transcript (“BMS-CDC2A”) were both used as non-specific negative controls. Percent knockdown was determined by measuring transcript levels in transfected versus non-transfected HeLa cells using RT-PCR as described in Example 6.

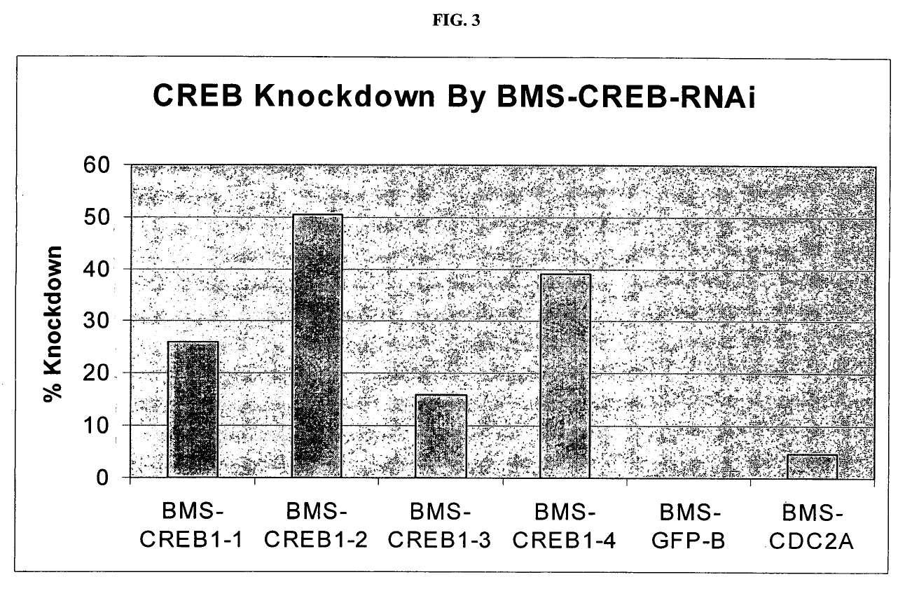

FIG. 4 shows transfection of 2×10⁵ HeLa cells with one of four RNAi reagents (“BMS-CREB1-1”; “BMS-CREB1-2”; “BMS-CREB1-3”; or “BMS-CREB1-4”) designed to target the CREB receptor transcript results in significant knock-down of CREB protein levels. HeLa cells transfected with a RNAi reagent specific to GFP B transcript (“CREB-Negative control”), and an RNAi reagent specific to CDC2A transcript (“BMS-CDC2A”) were both used as non-specific negative controls. Percent knockdown was determined by measuring protein levels in transfected versus non-transfected HeLa cells by quantifying the band intensity as described in Example 7.

FIG. 5 shows transfection of 2×10⁵ HeLa cells with one RNAi reagent (“BMS-CDC2A”) designed to target the cell division cycle 2, G1 to S and G2 to M protein (CDC2A; Genbank Accession No. gi|NM_{—}001786) transcript results in significant knock-down of CDC2A transcript levels. HeLa cells transfected with a RNAi reagent specific to GFP B transcript (“BMS-GFP-B”), and an RNAi reagent specific to CREB transcript (“BMS-CREB1-4”) were both used as non-specific negative controls. Percent knockdown was determined by measuring transcript levels in transfected versus non-transfected HeLa cells using RT-PCR as described in Example 8.

FIG. 6 shows the percent of A549 cells exhibiting nuclear fragmentation and/or swelling in response to transfection with one of the four E2F1-directed RNAi reagents disclosed herein (“E2F1-1”; “E2F1-2”; “E2F1-3”; “E2F1-4”). RNAi reagent directed against the Luciferase-4 (“Luc-4”) served as a negative control; while cells subjected to Lipofectamine 2000 alone (“LF2K”) and wells receiving no treatment (“no treatment”) were included to monitor transfection toxicity. As shown, transfection of A549 cells with RNAi reagents directed against E2F1 resulted in a significant increase in the number of cells exhibiting nuclear fragmentation and/or swelling relative to the controls, which is consistent with induction of apoptosis as a consequence of E2F1 downregulation. The experiments were performed as described in Example 9.

FIG. 7 shows histograms of DNA cell content in A549 cells transfected with either an E2F1-directed RNAi reagent (“E2F1-3”), or an RNAi reagent directed against Luciferase-4 (“Luc-4”) as determined by measuring intensity of DAPI in duplicate. Positions of diploid (“2N”) and double diploid (“4N”) are clearly indicated. The location of fragmented DNA in the cells treated with the E2F1-directed RNAi reagent E2F1-3, is labeled and indicated by an arrow. As shown, RNAi reagents directed against E2F1 resulted in a large increase in the G2/M cell population compared to the Luc-4 controls providing additional evidence that E2F1 is downregulated in response to transfection with E2F1-directed RNAi reagents. Additionally, the results show that the majority of the G2/M population of cells contain two nuclei which is an indication of a cytokinesis defect as a consequence of E2F1 downregulation. The experiments were performed as described in Example 10.

FIG. 8 shows immunocytochemistry images of A549 cells stained with TOTO-3, DAPI, and anti-α-tubulin 72 hours after transfection with either an E2F1-directed RNAi reagent (“E2F1-3”), or an RNAi reagent directed against the Luciferase-4 (“Luc-4”). Cells were subjected to Alexa-488 goat anti-rabbit IgG post primary antibody staining. Top slides show the A549 cells with dark field illumination, while the lower slides show the A549 cells under fluorescence. Cell nuclei stain blue with DAPI treatment, while α-tubulin stains green. As shown, transfection of A549 cells with RNAi reagents directed against E2F1 results in a significant increase in the number of cells exhibiting nuclear fragmentation and/or swelling relative to the Luc-4 controls, which is consistent with induction of apoptosis as a consequence of E2F1 downregulation. A representative diploid cell is denoted by a red arrow in the E2F1-3 RNAi treated cells. The experiments were performed as described in Example 9.

FIG. 9 shows a quantitative summary of the results illustrated and described in FIGS. 6, 7, and 8 for the four E2F1-directed RNAi reagents (“E2F1-1”; “E2F1-2”; “E2F1-3”; “E2F1-4”), as compared to RNAi reagent directed against the Luciferase-4 (“Luc-4”), cells subjected to Lipofectamine 2000 alone (“LF2K”), and wells receiving no treatment (“no treatment”). For these experiments, RNAi reagent directed against XIAP (X-linked Inhibitor of Apoptosis Protein) was used as a positive control (“XIAP”), since cells that lose XIAP undergo apoptosis. As shown, subjecting A549 cells with E2F1 RNAi reagent results in a significant decrease in the number of cells, in conjunction with a significant concomitant increase in the amount of caspase-3, α-tubulin, and TOTO-3 expression providing additional evidence that apoptosis is induced as a consequence of E2F1 downregulation.

FIG. 10 shows transfection of 2×10⁵ HeLa cells with one of four RNAi reagents (“BMS-E2F1-1”; “BMS-E2F1-2”; “BMS-E2F1-3”; or “BMS-E2F1-4”) designed to target the E2F1 receptor transcript (Genbank Accession No. gi|NM_{—}005225), results in significant downregulation of the Cyclin D1 gene. A fluorescently labeled RNAi reagent (“BMS-FITC-RNAi”) was used as a control to assess the transfection efficiency level. In addition, RNAi reagent specific to GFP B was used as a non-specific negative control (“BMS-GFP-B”). Percent knockdown was determined by measuring transcript levels in transfected versus GFP-B transfected HeLa cells using RT-PCR as described in Example 11.

FIG. 11 shows transfection of 2×10⁵ HeLa cells with one of four RNAi reagents (“BMS-E2F1-1”; “BMS-E2F1-2”; “BMS-E2F1-3”; or “BMS-E2F1-4”) designed to target the E2F1 receptor transcript (Genbank Accession No. gi|NM_{—}005225), results in significant downregulation of the cycle regulatory cdc2 kinase gene. A fluorescently labeled RNAi reagent (“BMS-FITC-RNAi”) was used as a control to assess the transfection efficiency level. In addition, RNAi reagent specific to GFP B was used as a non-specific negative control (“BMS-GFP-B”). Percent knockdown was determined by measuring transcript levels in transfected versus GFP-B transfected HeLa cells using RT-PCR as described in Example 12.

### DETAILED DESCRIPTION OF THE INVENTION

### DEFINITIONS

“RNAi reagent” is meant to encompass double-stranded nucleic acid molecules with high binding affinity for a particular targets nascent mRNA, and capable of silencing the gene target in a sequence specific manner. Also encompassed are the sense strand and antisense strand of each RNAi double stranded reagent and its use in silencing the gene target in a sequence specific manner. For the purposes of the present invention, “RNAi reagent” is used synonymously with the term “siRNA”.

The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of mediating RNA interference “RNAi” or gene silencing in a sequence-specific manner; see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056 15 60; McManus et al., 2002, RNA, 8, 842-850; Reinhart et al., 2002, Gene & Dev., 16, 1616-1626; and Reinhart & Bartel, 2002, Science, 297, 1831).

The term “nucleic acid” and “polynucleotide” are intended to encompass single stranded RNA, double stranded RNA, “RNAi reagents”, “short interfering nucleic acid”, “siNA”, “short interfering RNA”, “siRNA”, “short interfering nucleic acid molecule”, “short interfering oligonucleotide molecule”, “chemically-modified short interfering nucleic acid molecule”, in addition to any other nucleic acids disclosed or referenced herein that are capable of mediating RNA interference “RNAi” or gene silencing in a sequence-specific manner.

The term “acyclic nucleotide” as used herein refers to any nucleotide having an acyclic ribose sugar, for example where any of the ribose carbons (C1, C2, C3, C4, or C5), are independently or in combination absent from the nucleotide.

The term “downstream effectors” as used herein is meant to encompass any genes, polypeptides, and/or pathways, that may be directly or indirectly regulated by a transcription factor described herein, and/or an RNAi reagent described herein, wherein said genes, polypeptides, and/or pathways necessarily reside at a point downstream from the effect of said transcription factor and/or said RNAi reagent.

As used herein the terms “modulate” or “modulates” refer to an increase or decrease in the amount, quality or effect of a particular activity, DNA, RNA, or protein. The definition of “modulate” or “modulates” as used herein is meant to encompass agonists and/or antagonists of a particular activity, DNA, RNA, or protein.

Methods and compositions are provided for modulating gene expression in vitro, in vivo, and/or ex vivo. The methods involve administering a composition to a cell, tissue, and/or patient so as to introduce into a target cell molecular modulators comprising, for example, double-stranded nucleic acid, preferably RNA, more preferably an RNAi reagent, which is capable of downregulating the expression and/or activity of transcription factors thereby preventing them from binding to their cellular promoters and up or downregulating transcription as the case may be. Various methods are employed for in vitro, in vivo, and ex vivo administration of the RNAi's such that sufficient amounts enter into the target cells to inhibit transcription factor binding to an endogenous gene regulatory region, either directly or indirectly.

The compositions of the present invention preferably comprise RNAi reagents specific to transcription factors, which include, E2F1, and NFkB-p65. The targeted transcription factors are endogenous, sequence-specific double-stranded DNA binding proteins which modulate (e.g., increase or decrease) the rate of transcription of one or more specific genes in the target cell. Essentially, any transcription factor can be targeted so long as a specific RNAi capable of decreasing the transcript level of the transcription factor can be identified. Preferably, the RNAi reagent results in the effective inhibition of the transcription factors binding to one or more genes which are known to be modulated by the transcription factor. The latter is expected to be apparent if the transcript level of a particular transcription factor is decreased to a sufficient level so that the transcription factors intracellular protein levels are correspondingly decreased. Numerous transcription factors and their binding sequences are known in the art as are methods for identifying such complements, see e.g. Wang and Reed (1993) Nature 364, 121 and Wilson et al. (1991) Science 252, 1296. As used herein, endogenous means that the gene or transcription factor is present in the target cell at the time the RNAi is introduced.

The transcription factors will, for the most part and depending on the clinical indication, regulate the transcription of genes associated with cell growth, differentiation and signaling or viral genes resident in the target cell. Examples include genes necessary for mitosis, particularly going from G.sub.o to S, such as proteins associated with check points in the proliferative cycle, cyclins, cyclin dependent kinases, proteins associated with complexes, where the cyclin or cdk is part of the complex, Rosenblatt et al., Proc. Natl. Acad. Sci. 89, 2824 (1992) and Pagano et al., Science 255, 1144 (1992). Often such genes or the transcription factors themselves will be oncogene products or cellular counterparts, e.g. fos, jun, myc, etc. Other examples include genes encoding secreted proteins and peptides such as hormones e.g. growth factors, cytokines, e.g. interleukins, clotting factors, etc. Target transcription factors also include host and host-cell resident viral transcription factors which activate viral genes present in infected host cells.

Preferred target transcription factors are activated (i.e. made available in a form capable of binding DNA) in a limited number of specifically activated cells. For example, a stimulus such as a wound, allergen, infection, etc may activate a metabolic pathway that is triggered by the transient availability of one or more transcription factors. Such transcription factors may be made available by a variety of mechanisms such as release from sequestering agents or inhibitors (e.g. NF.kappa.B bound to IkB), activation by enzymes such as kinases, translation of sequestered message, etc. Desirably, the target transcription factor(s) will be associated with genes other than genes whose lack of expression results in cytotoxicity. For the most part, it is desirable not to kill the cell, but rather to inhibit or activate specific gene transcription.

Exemplary transcription factors and related cis elements, the cellular processes impacted and therapeutic indication include: E2F: cell proliferation, neointimal hyper-plasia, neoplasia glomerulonephritis, angiogenesis, inflammation: AP-1: cell growth, differentiation, neointimal hyper-growth factor expression plasia, cardiac myocyte growth/differentiation; NFkB: cytokine expression, leukocyte inflammation, immune adhesion molecule expression, response, transplant oxidant stress response, cAMP rejection, ischemia- and protein kinase C activation, reperfusion injury, Ig expression glomerulonephritis; SSRE: response to shear stress: growth neointimal hyper-factor expression vasoactive plasia, bypass grafts, substances, matrix proteins, angiogenesis, adhesion molecules, collateral formation; CREB: cAMP response, cAMP activated events, MEF-2 cardiac myocyte differentiation, cardiac myocyte, hypertrophy differentiation and growth; CarG: box cardiac myocyte differentiation, cardiac myocyte growth and differentiation, tax viral replication, HTLV infection, VP16 viral replication, Herpes infection, TAR/tat viral replication, HIV infection; GRE/HRE: glucocorticoid, mineralocorticoid, steroid hormone, MRE induced events processes e.g. (breast or prostate cell growth), heat shock, heat shock response, cellular stresses e.g. RE ischemia, hypoxia; SRE: growth factor responses, cell proliferation/ differentiation; AP-2: cAMP and protein kinase cell proliferation, retinoic acid response, sterol modulation of LDL cholesterol, hypercholesterolemia, response receptor expression element; TRE: transforming growth factor beta cell growth, TGFb induced cellular processes, entiation, migration, responsive angiogenesis, intimal element hyperplasia, matrix generation, and apoptosis.

The length, structure and nucleotide sequence of the RNAi will vary depending on the targeted transcription factor, the indication, route of administration, etc. Delivery may be as synthetically synthesized 15-50 bp double stranded RNAi or as 30-1000 base paired inverted repeats in a viral or plasmid vector which produce the RNAi molecules in vivo. Similarly, where transcription is mediated by a multimeric complex, it is often desirable to target a single transcription factor to minimize effects on non-targeted genes. For example, in the case of Herpes virus transcription, one may target the viral VP16 without concomitant targeting of the promiscuous host Oct protein.

In addition the RNAi's must be chosen for specificity. Desirably, the RNAi's will be highly specific for the target transcription factor(s) such that their effect on nontarget cells and nontargeted metabolic processes of target cells are minimized. Such selection is accomplished by genome blast programs to make sure that the chosen sequences are specific to the transcription factor in question and no other genes in the genome. In addition tests such as the upregulation of non-specific stress activated genes such as PKR genes and effects on transcripts of other genes that were not specifically targeted are monitored

The RNAi's contain sufficient nucleotide sequence to ensure target transcription factor binding specificity, specific degradation of the target transcript and binding of the Dicer Complex, and affinity sufficient for therapeutic effectiveness. For the most part, the target transcription factors will require at least 1 base pairs, usually at least about 19-50 base pairs for sufficient specificity and affinity. Frequently, providing the RNAi with flanking sequences (ranging from about 5 to 50 bp) enhance the knockdown or specificity. However the longer sequences in some circumstances can induce non-specific effects and these are monitored.

In one embodiment, the RNAi's are non-replicative oligonucleotides fewer than 100 bp, usually fewer than 50 bp and usually containing coding sequence or 5′ or 3′ UTR sequence which is primarily from the non-coding region of a gene. Alternatively, the RNAi's may comprise a portion of a larger plasmid, including viral vectors, capable of episomal maintenance or constitutive production of targeted double stranded RNAi in the target cell to provide longer term or enhanced intracellular exposure to the RNAi sequence. Plasmids are selected based on compatibility with the target cell, size and restriction sites, replicative frequency, copy number maintenance, etc. For example, plasmids with relatively short half-lives in the target cell are preferred in situations where it is desirable to maintain therapeutic transcriptional modulation for less than the lifetime of the target cell. Exemplary plasmids include pUC expression vectors driven by a beta-actin promoter and CMV enhancer, vectors containing elements derived from RSV or SV40 enhancers, etc. The adeno-associated viral vector preferentially integrates in chromosome 19 and may be utilized for longer term expression.

The oligonucleotides which are employed may be naturally occurring or other than naturally occurring, where the synthetic nucleotides may be modified in a wide variety of ways, see e.g. Bielinska et al (1990) Science 250, 997. Thus, oxygens may be substituted with nitrogen, sulfur or carbon; phosphorus substituted with carbon; deoxyribose substituted with other sugars, or individual bases substituted with an unnatural base. In each case, any change will be evaluated as to the effect of the modification on the binding of the oligonucleotide to the target transcription factor, as well as any deleterious physiological effects. These modifications have found wide application for “anti-sense” oligonucleotides, so that their safety and retention of binding affinity are well established in the literature. See, for example, Wagner et al., Science 260, 1510-1513 (1993). The strands may be synthesized in accordance with conventional ways using phosphoramidite synthesis, commercially available automatic synthesizers and commercially available RNA synthesis chemistry, and the like, or via other common chemistries.

The administered compositions may comprise individual or mixtures of RNAis. Usually the mixture will not exceed 2-4 different RNAi's. The RNAi's are administered to a host in a form permitting cellular internalization of the RNAi in an amount sufficient to result in the degradation of the targeted transcription factor and to downregulate its subsequent effects on endogenous genes. The host is typically a mammal, usually a human. The selected method of administration depends principally upon the target cell, the nature of the RNAi, the host, the size of the RNAi. Exemplary methods are described in the examples below; additional methods including transfection with a retrovirus, viral coat protein-liposome mediated transfection, lipofectin etc. are described in Dzau et al., Trends in Biotech 11, 205-210 (1993).

Where administered in liposomes, the RNAi concentration in the lumen will generally be in the range of about 0.001 uM to 50 uM per RNAi, more usually about 0.01 uM to 10 uM, most usually about 3 uM. For other techniques, usually one will determine the application rate empirically, using conventional techniques to determine desired ranges.

In some situations it may be desirable to provide the RNAi source with an agent which targets the target cells, such as an antibody specific for a surface membrane protein on the target cell, a ligand for a receptor on the target cell, etc. For example, for intervention in HIV infection, cells expressing HIV gene products or CD4 may be specifically targeted with gene product or CD4-specific binding compounds. Also, where liposomes are involved, one may wish to include proteins associated with endocytosis, where the proteins bind to a surface membrane protein associated with endocytosis. Thus, one may use capsid proteins or fragments thereof tropic for a particular cell type, antibodies for proteins that undergo internalization in cycling, and proteins that target intracellular localization and enhance intracellular half-life.

The application of the subject therapeutics are preferably local, so as to be restricted to a histological site of interest e.g. localized inflammation, neoplasia or infection. Various techniques can be used for providing the subject compositions at the site of interest, such as injection, use of catheters, trocars, projectiles, pluronic gel, stents, sustained drug release polymers or other device which provides for internal access, or the like. Where an organ or tissue is accessible because of removal from the patient, such organ or tissue may be bathed in a medium containing the subject compositions, the subject compositions may be painted onto the organ, or may be applied in any convenient way. Alternatively, systemic administration of the RNAi using, e.g. lipofection, liposomes with tissue targeting (e.g. antibody), etc. may be practiced. Systemic administration is most applicable where the distribution of the targeted transcription factor is primarily limited to targeted cell types, e.g. virus-specific transcription factors limited to infected cells, mutant oncogenic transcription factors limited to transformed cells, etc.

Optimal treatment parameters will vary with the indication, RNAi, clinical status, etc., and are generally determined empirically, using the guidance provided herein. Several exemplary indications, routes and vehicles of administration and RNAi combinations are disclosed in the following table.

TABLE V

| INDICATION | ROUTE | VEHICLE | PLASMD/OLIGO |
|---|---|---|---|
| HIV infection | Intravenous inj. | gp160 in neutral | TAR containing oligo |
| | | liposomes | |
| solid tumor | Intratumoral inj. | Tumor specific Ab with | E2F |
| | | liposomes | |
| Inflammatory skin | topical | polymer | NFkB, E2F |
| diseases and dermatitis | | | |
| Hypercholesterolemia | Intravenous inj. Portal | Asialoglycoprotein | Responsive element to |
| | vein inj. | receptor targeting with | increase LDL receptors |
| | | liposomes | |
| vein bypass grafts | Topical/intralumina | Polymer, liposomes | E2F |
| Glomerulonephritis | Intravenous, intrarenal | Polymer, liposomes | E2F1, NFkB |
| Myocardial infarction | intracoronary | Liposomes, Polymer | NFkB, E2F1, AP-1 |
| organ transplant, esp. | Intravascular, ex vivo | Liposomes, Polymer | NFkB |
| cardiac/renal | | | |

A wide variety of indications may be treated, either prophylactically or therapeutically with the subject compositions. For example, prophylactic treatment may inhibit mitosis or proliferation or inflammatory reaction prior to a stimulus which would otherwise activate proliferation or inflammatory response, where the extent of proliferation and cellular migration may be undesirable. Similarly, a therapeutic application is provided by a situation where proliferation or the inflammatory response is about to be initiated or has already been initiated and is to be controlled.

The methods and compositions find use, particularly in acute situations, where the number of administrations and time for administration is relatively limited.

Conditions for treatment include such conditions as neoproliferative diseases including inflammatory disease states, where endothelial cells, inflammatory cells, glomerular cells may be involved, restenosis, where vascular smooth muscle cells are involved, myocardial infarction, where heart muscle cells may be involved, glomerular nephritis, where kidney cells are involved, hypersensitivity such as transplant rejection where hematopoietic cells may be involved, cell activation resulting in enhancement of expression of adhesion molecules where leukocytes are recruited, or the like. By administering the RNAi to the organ ex vivo prior to implantation and/or after implantation, upregulation of the adhesion molecules may be inhibited. Adhesion molecules include homing receptors, addressing, integrins, selecting, and the like.

Additional conditions that may be treated by the compositions of the present invention include, but are not limited to the following: disorders associated with E2F1, disorders associated with aberrant E2F1 activity and/or expression, cell cycle disorders, cell cycle disorders associated with aberrant function of the S-phase check point, disorders associated with p53-dependent apoptosis, disorders associated with p53-independent apoptosis, proliferative disorders, proliferative disorders of the pancreas, human pancreatic carcinoma, proliferative disorders of the lung, nonsmall-cell lung cancer, proliferative disorders of the colon, colon cancer, proliferative disorders of the skin, skin cancer, proliferative disorders of the stomach, proliferative disorders of the gastrointestinal system, gastric cancer, MDM2-dependent proliferative disorders, checkpoint kinase 2 related disorders, G1 cell cycle checkpoint disorders, G2 cell cycle checkpoint disorders, aberrant cell cycle checkpoint protein disorders, disorders associated with aberrant CDK2 protein expression and/or activity, proliferative disorders of the immune system, proliferative disorders of leukemic cells, malignant lymphoma, proliferative disorders of the ovary, epithelial ovarian tumors, neural disorders, neurodegenerative disorders, Alzheimers, disorders associated with aberrant amyloid-beta expression and/or activity, disorders associated with aberrant NFKB expression and/or activity, disorders associated with aberrant cytokine expression and/or activity, disorders associated with high levels of oxidant-free radicals, disorders associated with high levels of ultraviolet irradiation, inflammatory disorders, rheumatoid arthritis, aberrant immune cell development, aberrant immune cell growth, disorders associated with aberrant vascular endothelial growth factor C expression and/or activity, disorders associated with tumor lymphangiogenesis, tumor metastasis process, proliferative disorder of the breast, breast cancer, disorders associated with aberrant heregulin-beta 1 expression and/or activity, disorders associated with interleukin-1 beta activity and/or expression, disorders associated with aberrant angiogenic potential of tissues—particularly tumors, pancreatic adenocarcinoma, disorders associated with aberrant neutrophil migration, bone disorders, disorders associated with aberrant osteoblast differentiation, proliferative disorders of bone cells and tissues, osteosarcomas, disorders associated with aberrant expression and/or activity of bone morphogenic proteins (BMP) 4, disorders associated with aberrant expression and/or activity of BMP7, disorders associated with aberrant expression and/or activity of Cbfa1, disorders associated with aberrant osteoblast differentiation, autoimmune disorders, arthritis, asthma, septic shock, lung fibrosis, glomerulonephritis, atherosclerosis, AIDS, aberrant apoptosis, inappropriate immune cell development, delayed cell growth, disorders associated with aberrant expression and/or activity of cAMP-response element binding protein (CREB1), acute myeloid leukemia, reproductive disorders, spermatogenesis, major depressive disorder, neuropathies, Huntington's disease, disorders associated with aberrant N-cadherin expression and/or activity, disorders associated with aberrant G-protein coupled receptor regulation and/or expression, pain disorders, and chronic pain.

Validation of RNAi reagents as therapeutic regiments, either alone or in combination with other therapeutic agents, is known in the art. For example, Acuity Pharmaceuticals has demonstrated the efficacy of an siRNA specific to the VEGF mRNA in the treatment of age-related macular degeneration. The VEGF siRNA was able to significantly inhibit both the blood vessel overgrowth (neovascularization) and vascular leakage that are integral components leading to the incidence of AMD in a primate disease model. At the highest dose used in the study the VEGF siRNA reduced the incidence of clinically significant vascular leakage to zero by week three and for the duration of the study, and at day 35 neovascularization was inhibited by greater than 65 percent in the high dose group. The siRNA was believed to inhibit VEGF expression at levels from 100 to 1000 times greater than that observed with other treatment regimens directly against VEGF (Tolentino, et al., “Intravitreal injection of VEGF siRNA Inhibits growth and leakage in a non-human primate laser induced model of CNV”, February 2004 issue of the journal Retina, the Journal of Retinal and Vitreous Diseases; which is hereby incorporated herein by reference in its entirety; and PCT International Publication No. WO0409769, filed Jul. 18, 2003; which is hereby incorporated herein by reference in its entirety) See also Reich S J et al., Mol Vis. 2003 May 30;9:210-6.

Additional methods, processes, and uses are disclosed in U.S. Ser. No. US20020052333, filed on Apr. 19, 2001; and U.S. Ser. No. US20020128217, filed on Jun. 5, 2001; which are hereby incorporated by reference herein in their entirety.

### E2F1-Directed siRNA Reagents

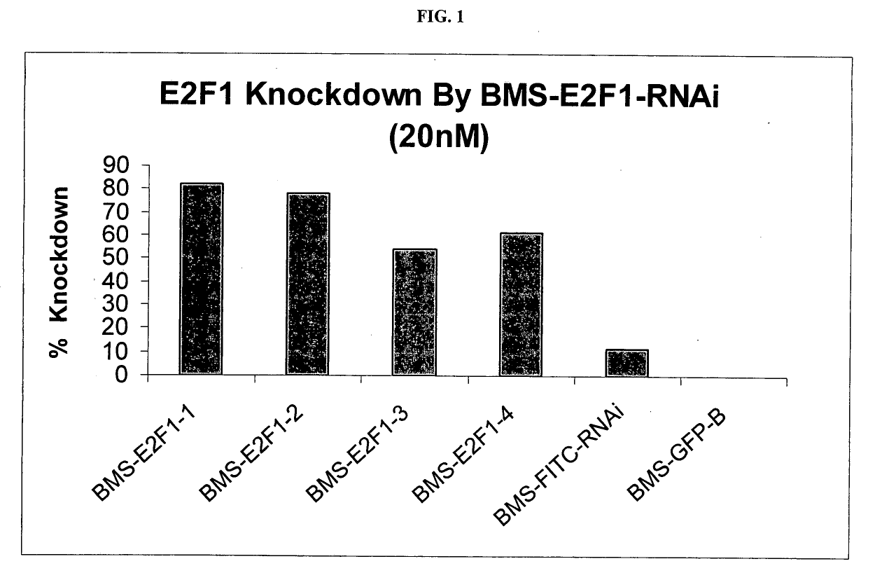

The E2F1-directed siRNA reagents of the present invention, namely BMS-E2F1-1, BMS-E2F1-2, BMS-E2F1-3, and BMS-E2F1-4, have been shown herein to directly downregulate the level of E2F1 expressed in cells transfected each of these reagents (see FIG. 1).

As a consequence, the E2F1-directed RNAi reagents have a number of uses which include, but are not limited to: disorders associated with E2F1, disorders associated with aberrant E2F1 activity and/or expression, cell cycle disorders, cell cycle disorders associated with aberrant function of the S-phase check point, disorders associated with p53-dependent apoptosis, disorders associated with p53-independent apoptosis, proliferative disorders, proliferative disorders of the pancreas, human pancreatic carcinoma, proliferative disorders of the lung, nonsmall-cell lung cancer, proliferative disorders of the colon, colon cancer, proliferative disorders of the skin, skin cancer, proliferative disorders of the stomach, proliferative disorders of the gastrointestinal system, gastric cancer, MDM2-dependent proliferative disorders, checkpoint kinase 2 related disorders, G1 cell cycle checkpoint disorders, G2 cell cycle checkpoint disorders, aberrant cell cycle checkpoint protein disorders, disorders associated with aberrant CDK2 protein expression and/or activity, proliferative disorders of the immune system, proliferative disorders of leukemic cells, malignant lymphoma, proliferative disorders of the ovary, and epithelial ovarian tumors.

Moreover, the E2F1-directed RNAi reagents are also useful for the treatment, amelioration, and/or prevention of: restenosis, restenosis of vascular smooth muscle cells, restenosis resulting from neointima formation, neointimal hyperplasia, neoplasia glomerulonephritis, angiogenesis, inflammation and proliferative lesions in a blood vessel.

Characterization of the E2F1-directed RNAi reagents of the present invention led to the discovery that cells transfected with each of these reagents not only results in downregulation of E2F1, but also to apoptosis and cell cycle disruption. These results directly support the use of these reagents for treating, preventing, and/or ameliorating proliferative disorders, and other E2F1-associated disorders described herein.

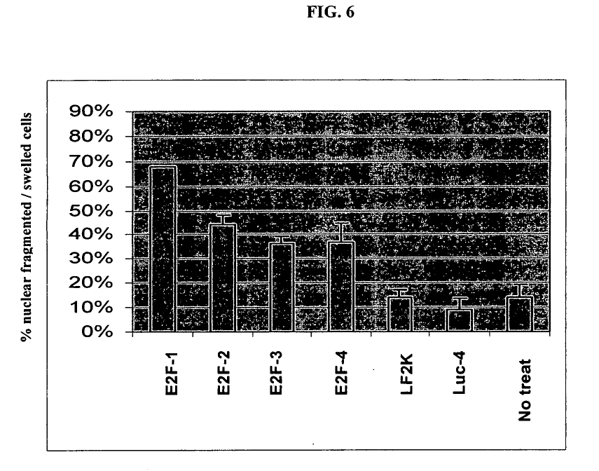

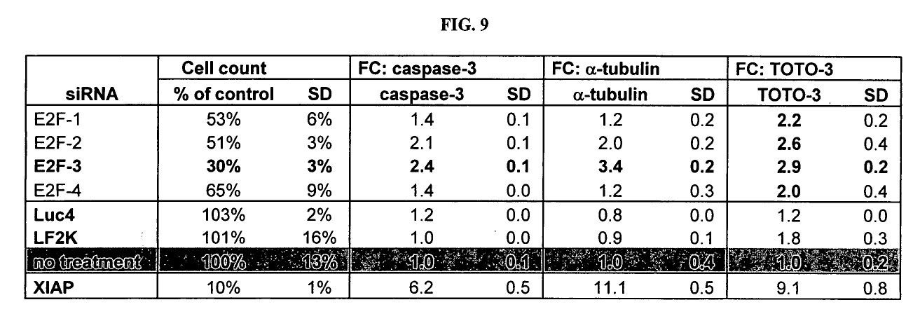

Specifically, experiments designed to assess the effect of transfecting A549 cells with one of the four E2F1-directed RNAi reagents disclosed herein (“E2F1-1”; “E2F1-2”; “E2F1-3”; “E2F1-4”) were performed (see FIGS. 6 and 9). The results demonstrated that cells transfected with the E2F1-directed RNAi reagents exhibited significant nuclear fragmentation and/or swelling relative to control cells.

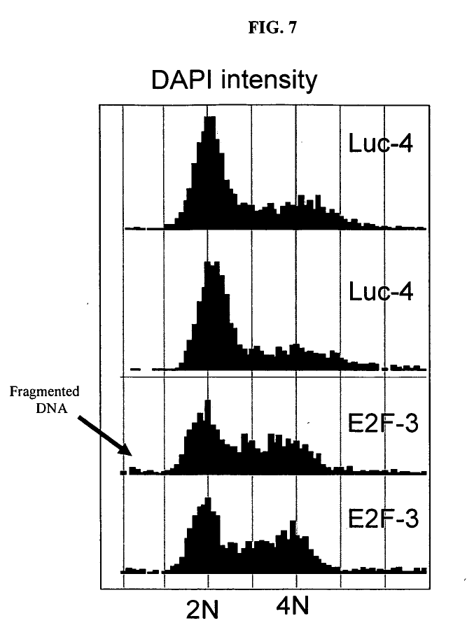

Further analysis of A549 cells transfected with one of the four E2F1-directed RNAi reagents demonstrated that there was a significant increase in the number of cells in the G2/M phase of the cell cycle, relative to control cells (see FIGS. 7 and 9). Additionally, the results also showed that the majority of the G2/M population of cells contained two nuclei which is an indication of a cytokinesis defect as a consequence of E2F1 downregulation.

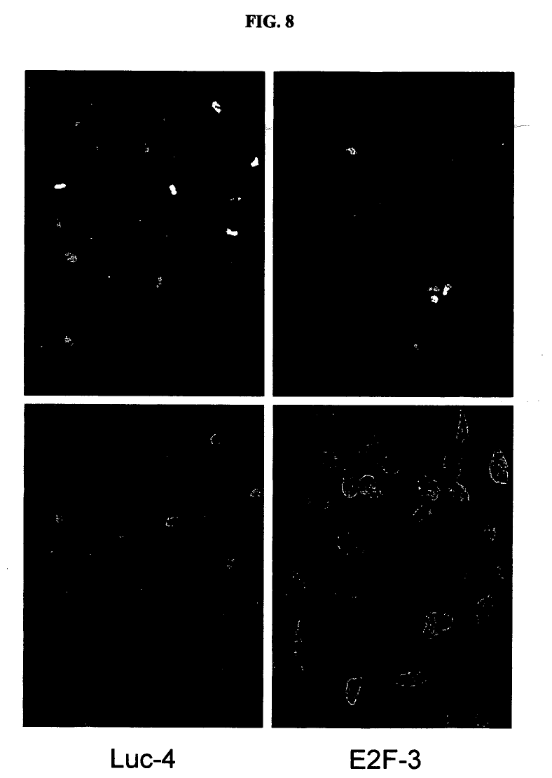

Immunocytochemistry images of A549 cells stained with the cell membrane impermeable dye TOTO-3, the minor groove DNA binding dye DAPI, and anti-x-tubulin 72 hours after transfection with a E2F1-directed RNAi reagent showed a significant increase in the number of cells exhibiting nuclear fragmentation and/or swelling relative to controls (see FIGS. 8 and 9).

Each of these experiments clearly demonstrates that the E2F1-directed RNAi reagents not only downregulate E2F1, but also induce apoptosis and inhibit cellular proliferation at the G2/M cell cycle checkpoint.

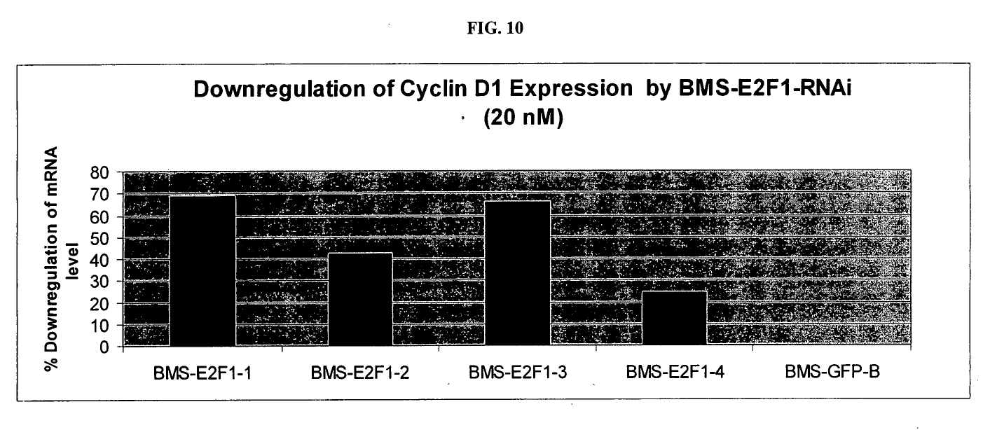

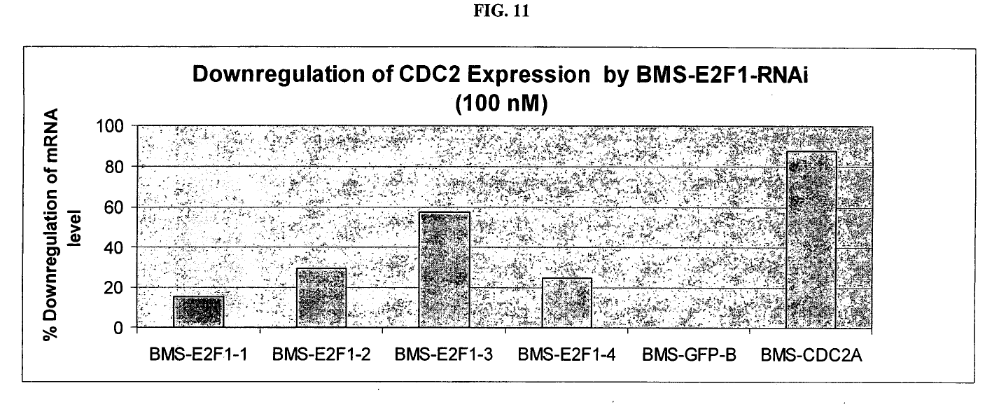

Additional experiments demonstrated that transfection of cells with one of the E2F1-directed RNAi reagents also resulted in significant downregulation of the Cyclin D1 gene (see FIG. 10), in addition to significant downregulation of the cycle regulatory cdc2 kinase gene (see FIG. 11), providing additional evidence of these reagents ability to disrupt cell cycle regulation.

These results are consistent with results obtained for E2F1-directed decoy oligonucleotides that are currently in clinical trials for the treatment of vein-graft restinosis (see U.S. Ser. No. US20020052333, filed on Apr. 19, 2001; and U.S. Ser. No. US20020128217, filed on Jun. 5, 2001).

Although it is well known that overexpression of E2F1 in cells results in the induction of apoptosis, downregulation of E2F1 has also been shown to induce apoptosis with the latter supported by the instant teachings in addition to the use of the E2F1-directed decoy oligonucleotides described supra. Moreover, Wikonkal N. M., et al (Nat Cell Biol. July 2003;5(7):587-9; and Nat Cell Biol. July 2004;6(7):565-7) have also demonstrated that E2F1 functions as an apoptosis pathway suppressor based upon experiments showing that E2f1−/− mice have increased levels of apoptosis after UVB exposure, which is repressed upon transfecting E2F1 into E2f1−/− cells.

### Polynucleotides

The present invention relates to a nucleic acid molecules that act as mediators of the RNA interference gene silencing response. Preferably, such molecules are double-stranded nucleic acid molecules. In one embodiment, the nucleic acid molecules of the present invention consist of duplexes containing about 19 base pairs between oligonucleotides comprising about 19 to about 25 nucleotides. In this context, the term “about” may be construed to represent 19, 20, 21, 22, 23, 24 or 25 nucleotides in each oligonucleotide. In yet another embodiment, the nucleic acid molecules of the present invention comprise duplexes with overhanging nucleotide ends of about 1 to about 3 nucleotides in length. In this context, the term “about” may be construed to represent 1, 2, 3, 4, 5, 6, or more nucleotides in length, and preferably 1, 2, or 3 nucleotides in length. In one embodiment, the nucleic acid molecules are 21 nucleotide duplexes with about 19 base pairs and 3′-terminal mononucleotide, dinucleotide, or trinucleotide overhangs on one or both oligonucleotides.

The present invention relates to a nucleic acid from about 8 to about 30 nucleotides in length, preferably from about 15 to about 25 nucleotides in length, more preferably from about 19 to about 23 nucleotides in length. In this context, the term “about” may be construed to represent 1, 2, 3, 4, 5, or 6 nucleotides more in either the 5′ or 3′ direction.

The present invention provides a polynucleotide comprising, or alternatively consisting of, the sequence identified as a member of the group consisting of: SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, and/or 35.

The present invention also provides polynucleotides encoding a polypeptide comprising, or alternatively consisting the sequence identified as a member of the group consisting of: SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, and/or 35, wherein said polynucleotide hybridizes to the coding region of the E2F1, NFkB, CREB-1, or CDC2A polypeptide.

The present invention also provides polynucleotides encoding a polypeptide comprising, or alternatively consisting the sequence identified as a member of the group consisting of: SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, and/or 35, wherein said polynucleotide hybridizes to the coding region of the E2F1, NFkB, CREB-1, or CDC2A polypeptide, wherein said coding region comprises one or more polymorphisms.

The present invention also provides polynucleotides encoding a polypeptide comprising, or alternatively consisting the sequence identified as a member of the group consisting of: SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, and/or 35, wherein said polynucleotide comprises one or more conservative nucleotide substitutions that are capable of hybridizing to the coding region of the E2F1, NFkB, CREB-1, or CDC2A polypeptide, wherein said coding region comprises one or more polymorphisms.

The present invention also provides polynucleotides comprising one or more chemically-modified nucleic acids having specificity for E2F1, NFkB, CREB-1, or CDC2A expressing nucleic acid molecules, such as RNA encoding E2F1, NFkB, CREB-1, or CDC2A protein. Non-limiting examples of such chemical modifications include without limitation phosphorothioate inten1ucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation. These chemical modifications, when used in various nucleic acids of the present invention, may preserve RNAi activity in cells while at the same time, dramatically increasing the serum stability of these compounds. Additional chemical modifications are provided elsewhere herein.

The present invention also encompasses polynucleotides capable of hybridizing, preferably under reduced stringency conditions, more preferably under stringent conditions, and most preferably under highly stringent conditions, to polynucleotides described herein. Examples of stringency conditions are shown in Table VI below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE VI

| | | | Hybridization | |
|---|---|---|---|---|
| Stringency | Polynucleotide | Hybrid Length | Temperature and | Wash Temperature |
| Condition | Hybrid± | (bp)‡ | Buffer† | and Buffer† |
| A | DNA:DNA | > or equal to 50 | 65° C.; 1xSSC - | 65° C.; |
| | | | or- 42° C.; | 0.3xSSC |
| | | | 1xSSC, 50% | |
| | | | formamide | |
| B | DNA:DNA | <50 | Tb*; 1xSSC | Tb*; 1xSSC |
| C | DNA:RNA | > or equal to 50 | 67° C.; 1xSSC - | 67° C.; |
| | | | or- 45° C.; | 0.3xSSC |
| | | | 1xSSC, 50% | |
| | | | formamide | |
| D | DNA:RNA | <50 | Td*; 1xSSC | Td*; 1xSSC |
| E | RNA:RNA | > or equal to 50 | 70° C.; 1xSSC - | 70° C.; |
| | | | or- 50° C.; | 0.3xSSC |
| | | | 1xSSC, 50% | |
| | | | formamide | |
| F | RNA:RNA | <50 | Tf*; 1xSSC | Tf*; 1xSSC |
| G | DNA:DNA | > or equal to 50 | 65° C.; 4xSSC - | 65° C.; |
| | | | or- 45° C.; | 1xSSC |
| | | | 4xSSC, 50% | |
| | | | formamide | |
| H | DNA:DNA | <50 | Th*; 4xSSC | Th*; 4xSSC |
| I | DNA:RNA | > or equal to 50 | 67° C.; 4xSSC - | 67° C.; |
| | | | or- 45° C.; | 1xSSC |
| | | | 4xSSC, 50% | |
| | | | formamide | |
| J | DNA:RNA | <50 | Tj*; 4xSSC | Tj*; 4xSSC |
| K | RNA:RNA | > or equal to 50 | 70° C.; 4xSSC - | 67° C.; |
| | | | or- 40° C.; | 1xSSC |
| | | | 6xSSC, 50% | |
| | | | formamide | |
| L | RNA:RNA | <50 | T1*; 2xSSC | Tl*; 2xSSC |
| M | DNA:DNA | > or equal to ° C., | 50° C.; 4xSSC - | 50° C.; |
| | | | or- 40° C. 6xSSC, | 2xSSC |
| | | | 50% formamide | |
| N | DNA:DNA | <50 | Tn*; 6xSSC | Tn*; 6xSSC |
| O | DNA:RNA | > or equal to 50 | 55° C.; 4xSSC - | 55° C.; |
| | | | or- 42° C.; | 2xSSC |
| | | | 6xSSC, 50% | |
| | | | formamide | |
| P | DNA:RNA | <50 | Tp*; 6xSSC | Tp*; 6xSSC |
| Q | RNA:RNA | > or equal to 50 | 60° C.; 4xSSC - | 60° C.; |
| | | | or- 45° C.; | 2xSSC |
| | | | 6xSSC, 50% | |
| | | | formamide | |
| R | RNA:RNA | <50 | Tr*; 4xSSC | Tr*; 4xSSC |
| ‡The “hybrid length” is the anticipated length for the hybridized region(s) of the hybridizing polynucleotides. When hybridizing a polynucleotide of unknown sequence, the hybrid is assumed to be that of the hybridizing polynucleotide of the present invention. When polynucleotides of | | | | |
| # known sequence are hybridized, the hybrid length can be determined by aligning the sequences of the polynucleotides and identifying the region or regions of optimal sequence complementarity. Methods of aligning two or more polynucleotide sequences and/or determining the percent identity between | | | | |
| # two polynucleotide sequences are well known in the art (e.g., MegAlign program of the DNA*Star suite of programs, etc). | | | | |
| †SSPE (1xSSPE is 0.15M NaCl, 10 mM NaH2PO4, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete. The hybridizations and washes may | | | | |
| # additionally include 5X Denhardt's reagent, .5-1.0% SDS, 100 ug/ml denatured, fragmented salmon sperm DNA, 0.5% sodium pyrophosphate, and up to 50% formamide. | | | | |
| *Tb-Tr: The hybridization temperature for hybrids anticipated to be less than 50 base pairs in length should be 5-10° C. less than the melting temperature Tm of the hybrids there Tm is determined according to the following equations. For hybrids less than 18 base pairs in length, | | | | |
| # Tm(° C.) = 2(# of A + T bases) + 4(# of G + C bases). For hybrids between 18 and 49 base pairs in length, Tm(° C.) = 81.5 + 16.6(log₁₀[Na+]) + 0 where N is the number of bases in the hybrid, and [Na+] is the concentration of sodium ions in the hybridization buffer ([NA+] for 1xSSC = .165 M). | | | | |

±The present invention encompasses the substitution of any one, or more DNA or RNA hybrid partners with either a PNA, or a modified polynucleotide. Such modified polynucleotides are known in the art and are more particularly described elsewhere herein.

Additional examples of stringency conditions for polynucleotide hybridization are provided, for example, in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M., Ausubel et al., eds, John Wiley and Sons, Inc., sections 2.10 and 6.3-6.4, which are hereby incorporated by reference herein.

Preferably, such hybridizing polynucleotides have at least 70% sequence identity (more preferably, at least 80% identity; and most preferably at least 90% or 95% identity) with the polynucleotide of the present invention to which they hybridize, where sequence identity is determined by comparing the sequences of the hybridizing polynucleotides when aligned so as to maximize overlap and identity while minimizing sequence gaps. The determination of identity is well known in the art, and discussed more specifically elsewhere herein.

The invention encompasses the application of PCR methodology to the polynucleotide sequences of the present invention. PCR techniques for the amplification of nucleic acids are described in U.S. Pat. No. 4,683,195 and Saiki et al., Science, 239:487-491 (1988). PCR, for example, may include the following steps, of denaturation of template nucleic acid (if double-stranded), annealing of primer to target, and polymerization. The nucleic acid probed or used as a template in the amplification reaction may be genomic DNA, cDNA, RNA, or a PNA. PCR may be used to amplify specific sequences from genomic DNA, specific RNA sequence, and/or cDNA transcribed from mRNA. References for the general use of PCR techniques, including specific method parameters, include Mullis et al., Cold Spring Harbor Symp. Quant. Biol., 51:263, (1987), Ehrlich (ed), PCR Technology, Stockton Press, NY, 1989; Ehrlich et al., Science, 252:1643-1650, (1991); and “PCR Protocols, A Guide to Methods and Applications”, Eds., Innis et al., Academic Press, New York, (1990).

The present invention encompasses polynucleotides with sequences complementary to those of the polynucleotides of the present invention disclosed herein. Such sequences may be complementary to the sequence disclosed as SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, and/or 35.

### Polynucleotide Variants

The present invention also encompasses variants (e.g., sequences containing conservative nucleotide substitutions, sequences containing nucleotide substitutions that are capable of hybridizing to known allelic variants of E2F1, NFkB, CREB-1, and/or CDC2A, fragments, sequences containing appropriate nucleotide substitutions such that they are capable of hybridizing to orthologs of E2F1, NFkB, CREB-1, and/or CDC2A, etc.) of the polynucleotide sequence disclosed herein in SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, and/or 35, and/or the complementary strand thereto.

“Variant” refers to a polynucleotide differing from the polynucleotide or polypeptide of the present invention, but retaining essential properties thereof (e.g., retaining ability to hybridize to the coding region of the E2F1, NFkB, CREB-1, and/or CDC2A polypeptides). Generally, variants are overall closely similar, and, in many regions, identical to the polynucleotide of the present invention.

Thus, one aspect of the invention provides an isolated nucleic acid molecule comprising, or alternatively consisting of, a polynucleotide having a nucleotide sequence selected from the group consisting of: (a) a sequence selected from the group consisting of: SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, and/or 35; (b) a sequence from “a” containing conservative nucleotide substitutions; (c) a sequence from “a” containing nucleotide substitutions that are capable of hybridizing to known allelic variants of E2F1, NFkB, CREB-1, and/or CDC2A; (d) fragments of “a”; (e) a sequence from “a” containing appropriate nucleotide substitutions such that they are capable of hybridizing to orthologs of E2F1, NFkB, CREB-1, and/or CDC2A; (f) a sequence from “a” that represents the complimentary strand; (g) a sequence from “a” that represents the sense strand; and/or (h) a sequence from “a” which is double stranded RNA.

The present invention is also directed to polynucleotide sequences which comprise, or alternatively consist of, a polynucleotide sequence which is at least about 80%, 85%, 90%, 91%, 92%, 93%, 93.6%, 94%, 95%, 96%, 97%, 98%, 99%, 99.1%, 99.2%, 99.3%, 99.4%, 99.5%, 99.6%, 99.7%, 99.8%, or 99.9% identical to, for example, any of the nucleotide sequences in (a), (b), (c), (d), (e), (f), (g), or (h), above. Polynucleotides encoded by these nucleic acid molecules are also encompassed by the invention. In another embodiment, the invention encompasses nucleic acid molecules which comprise, or alternatively, consist of a polynucleotide which hybridizes under stringent conditions, or alternatively, under lower stringency conditions, to a polynucleotide in (a), (b), (c), (d), (e), (f), (g), or (h),) above. Polynucleotides which hybridize to the complement of these nucleic acid molecules under stringent hybridization conditions or alternatively, under lower stringency conditions, are also encompassed by the invention, as are polypeptides encoded by these polypeptides.

By a nucleic acid having a nucleotide sequence at least, for example, 95% “identical” to a reference nucleotide sequence of the present invention, it is intended that the nucleotide sequence of the nucleic acid is identical to the reference sequence except that the nucleotide sequence may include up to five point mutations per each 100 nucleotides of the reference nucleotide sequence encoding the polypeptide. In other words, to obtain a nucleic acid having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence provided in SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, and/or 35, or any fragment specified as described herein.

As a practical matter, whether any particular nucleic acid molecule is at least about 80%, 85%, 90%, 91%, 92%, 93%, 93.6%, 94%, 95%, 96%, 97%, 98%, 99%, 99.1%, 99.2%, 99.3%, 99.4%, 99.5%, 99.6%, 99.7%, 99.8%, or 99.9% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992). In a sequence alignment the query and subject sequences are both DNA sequences. An RNA sequence can be compared by converting U's to T's. However, the CLUSTALW algorithm automatically converts U's to T's when comparing RNA sequences to DNA sequences. The result of said global sequence alignment is in percent identity. Preferred parameters used in a CLUSTALW alignment of DNA sequences to calculate percent identity via pairwise alignments are: Matrix=IUB, k-tuple=1, Number of Top Diagonals=5, Gap Penalty=3, Gap Open Penalty 10, Gap Extension Penalty=0.1, Scoring Method=Percent, Window Size=5 or the length of the subject nucleotide sequence, whichever is shorter. For multiple alignments, the following CLUSTALW parameters are preferred: Gap Opening Penalty=10; Gap Extension Parameter=0.05; Gap Separation Penalty Range=8; End Gap Separation Penalty=Off; % Identity for Alignment Delay=40%; Residue Specific Gaps:Off; Hydrophilic Residue Gap=Off; and Transition Weighting=0. The pairwise and multple alignment parameters provided for CLUSTALW above represent the default parameters as provided with the AlignX software program (Vector NTI suite of programs, version 6.0).

The present invention encompasses the application of a manual correction to the percent identity results, in the instance where the subject sequence is shorter than the query sequence because of 5′ or 3′ deletions, not because of internal deletions. If only the local pairwise percent identity is required, no manual correction is needed. However, a manual correction may be applied to determine the global percent identity from a global polynucleotide alignment. Percent identity calculations based upon global polynucleotide alignments are often preferred since they reflect the percent identity between the polynucleotide molecules as a whole (i.e., including any polynucleotide overhangs, not just overlapping regions), as opposed to, only local matching polynucleotides. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the CLUSTALW sequence alignment. This percentage is then subtracted from the percent identity, calculated by the above CLUSTALW program using the specified parameters, to arrive at a final percent identity score. This corrected score may be used for the purposes of the present invention. Only bases outside the 5′ and 3′ bases of the subject sequence, as displayed by the CLUSTALW alignment, which are not matched/aligned with the query sequence, are calculated for the purposes of manually adjusting the percent identity score.

The variants may contain alterations in the coding regions, non-coding regions, or both. Especially preferred are polynucleotide variants containing alterations which produce silent substitutions, additions, or deletions, but do not alter the properties or activities of the polynucleotide of the present invention. Nucleotide variants produced by silent substitutions due to the degeneracy of the genetic code are preferred. Moreover, nucleotide variants that correspond to the coding region of either E2F1, NFkB, CREB-1, and/or CDC2A in which 5-10, 1-5, or 1-2 amino acids are substituted, deleted, or added in any combination are also preferred. Polynucleotide variants can be produced for a variety of reasons, e.g., to optimize hybridization to an allelic variant or ortholog of E2F1, NFkB, CREB-1, and/or CDC2A, etc.).

Naturally occurring variants are called “allelic variants” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level and are included in the present invention. Alternatively, non-naturally occurring variants may be produced by mutagenesis techniques or by direct synthesis.

The present invention also encompasses polynucleotide variants that represent 5′-terminal or 3′-terminal deletion mutants. Deletion mutants of the present invention preferably comprise 1, 2, 3, 4, 5, 6, 7, 8, 9, 10, 11, 12, 13, 14, 15, 16, 17, 19, or 20 nucleotide deletions at the 5′ end of the polynucleotide; comprise 1, 2, 3, 4, 5, 6, 7, 8, 9, 10, 11, 12, 13, 14, 15, 16, 17, 19, or 20 nucleotide deletions at the 3′ end of the polynucleotide; or comprise a combination of 5′- and 3′-terminal deletions.

The present invention also encompasses polynucleotide variants that comprise one or more additional nucleotides at either the 5′-terminal or 3′-terminal end of the polynucleotide. Mutants of the present invention preferably comprise 1, 2, 3, 4, 5, 6, 7, 8, 9, 10, 11, 12, 13, 14, 15, 16, 17, 19, or 20 additional nucleotides at the 5′ end of the polynucleotide; comprise 1, 2, 3, 4, 5, 6, 7, 8, 9, 10, 11, 12, 13, 14, 15, 16, 17, 19, or 20 additional nucleotides at the 3′ end of the polynucleotide; or comprise a combination of additional nucleotides at either the 5′- and 3′-terminal end. The additional nucleotides to be added to the polynucleotides of the present invention may be determined by mapping the location of where the polynucleotide would be expected to hybridize to the coding region of either the E2F1, NFkB, CREB-1, and/or CDC2A by using a sequence alignment program (e.g., CLUSTALW), and determining the identity of however many nucleotides are indented to be added in either the 5′ or 3′ direction, and adding these nucleotides to the sequence.

As discussed supra, the present invention encompasses polynucleotides having a lower degree of identity but having sufficient similarity so as to still hybridize to the coding region of either E2F1, NFkB, CREB-1, and/or CDC2A and inhibit the expression and/or activity of E2F1, NFkB, CREB-1, and/or CDC2A. Similarity may be determined by conserved amino acid substitution of the encoded polypeptide. Such substitutions are those that substitute a given amino acid in a polypeptide by another amino acid of like characteristics (e.g., chemical properties). According to Cunningham et al above, such conservative substitutions are likely to be phenotypically silent. Additional guidance concerning which amino acid changes are likely to be phenotypically silent are found in Bowie et al., Science 247:1306-1310 (1 990).

Tolerated conservative amino acid substitutions of the encoding polynucleotides of the present invention involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

In addition, the present invention also encompasses the conservative substitutions provided in Table VII below.

TABLE VII

| For Amino | | |
|---|---|---|
| Acid | Code | Replace with any of: |
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-1-thioazolidine-4-carboxylic acid, D- |
| | | or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Both identity and similarity can be readily calculated by reference to the following publications: Computational Molecular Biology, Lesk, A. M., ed., Oxford University Press, New York, 1988; Biocomputing: Informatics and Genome Projects, Smith, D. W., ed., Academic Press, New York, 1993; Informatics Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991.

In addition, the present invention also encompasses substitution of nucleotides based upon the probability of an amino acid substitution resulting in conservation of hybridizational function. Such probabilities are determined by aligning multiple genes with related function and assessing the relative penalty of each substitution to proper gene function. Such probabilities are often described in a matrix and are used by some algorithms (e.g., BLAST, CLUSTALW, GAP, etc.) in calculating percent similarity wherein similarity refers to the degree by which one nucleotide may substitute for another nucleotide without lose of function. An example of such a matrix is the PAM250 or BLOSUM62 matrix.

Besides conservative nucleotide substitution, additional variants of the present invention include, but are not limited to, the following: (i) substitutions with one or more nucleotides that do not encode conserved amino acid residues, where the substituted amino acid residues may or may not be one encoded by the genetic code, or (ii) substitution with one or more nucleotide residues that have a substituent group, or (iii) fusion of polynucleotide to another compound, such as a compound to increase the stability and/or solubility of the polynucleotide. Such variant polypeptides are deemed to be within the scope of those skilled in the art from the teachings herein.

In one embodiment, a nucleic acid molecule of the present invention comprises modified nucleotides while maintaining the ability to mediate RNAi. The modified nucleotides can be used to improve in vitro or in viva characteristics such as ability, activity, and/or bioavailability. For example, a nucleic acid molecule of the present invention can comprise modified nucleotides as a percentage of the total number of nucleotides present in the nucleic acid molecule. As such, a nucleic acid molecule of the present invention can generally comprise about 5%, 10%, 15%, 20%, 25%, 30%, 35%, 40%, 45%, 50%, 55%, 60%, 65%, 70%, 75%, 80%, 85%, 90%, 95%, to about 100% modified nucleotides. In this context, the term “about” shall be construed to represent 1, 2, 3, 4, or 5% more or less modified nucleotides at each percent noted.

The actual percentage of modified nucleotides present in a given nucleic acid molecule will depend on the total number of nucleotides present in the nucleic acid. If the nucleic acid molecule is single stranded, the percent modification can be based upon the total number of nucleotides present in the single stranded nucleic acid molecules. Likewise, if the nucleic acid molecule is double stranded, the percent modification can be based upon the total number of nucleotides present in the sense strand, antisense strand, or both the sense and antisense strands.

In a non-limiting example, the introduction of chemically-modified nucleotides into nucleic acid molecules provides a powerful tool in overcoming potential limitations of in vivo stability and bioavailability inherent to native RNA molecules that are delivered exogenously. For example, the use of chemically-modified nucleic acids molecules can enable a lower dose of a particular nucleic acid molecule for a given RNAi effect, including therapeutic effects, since chemically-modified nucleic acids molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acids molecule is reduced as compared to a native nucleic acid molecule, for example, when compared to an all-RNA nucleic acid molecule, the overall activity of the modified nucleic acids molecule can be greater than that of the native molecule due to improved stability and/or delivery of the molecule. Unlike native unmodified nucleic acids, chemically-modified nucleic acids can also minimize the possibility of activating interferon activity in humans.

The antisense region of a nucleic acid molecule of the present invention can comprise a phosphorothioate internucleotide linkage at the -3′-end of said antisense region. The antisense region can comprise about one to about five phosphorothioate internucleotide linkages at the 5′-end of said antisense region. The 3′-terminal nucleotide overhangs of a nucleic acid molecule of the present invention can comprise ribonucleotides or deoxyribonucleotides that are chemically-modified at a nucleic acid sugar, base, or backbone. The 3′-terminal nucleotide overhangs can comprise one or more universal base ribonucleotides. The 3′-terminal nucleotide overhangs can comprise one or more acyclic nucleotides.

In one embodiment, the invention features a chemically-modified short interfering nucleic acid (nucleic acid) molecule capable of mediating RNA interference (RNAi) against either E2F1, NFkB, CREB-1, or CDC2A inside a cell or reconstituted in vitro system, wherein the chemical modification comprises one or more (e. g. about 1, 2, 3, 4, 5, 6, 7, 8, 9, 10, or more) nucleotides comprising a backbone modified internucleotide linkage having Formula I: 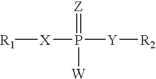
wherein each R1 and R2 is independently any nucleotide, non-nucleotide, or polynucleotide which can be naturally-occurring or chemically-modified, each X and Y is independently O, S, N, alkyl, or substituted alkyl, each Z and W is independently O, S, N, alkyl, substituted alkyl, O-alkyl, S-alkyl, alkaryl, or aralkyl, and wherein W, X, Y, and Z are optionally not all O. The chemically-modified internucleotide linkages having Formula I, for example, wherein any Z, W, X, and/or Y independently comprises a sulphur atom, can be present in one or both oligonucleotide strands of the nucleic acid duplex, for example, in the sense strand, the antisense strand, or both strands. The nucleic acid molecules of the present invention can comprise one or more (e.g. about 1, 2, 3, 4, 5, 6, 7, 8, 9, 10, or more) chemically modified internucleotide linkages having Formula I at the 3′-end, the 5′-end, or both of the 3′ and 5′-ends of the sense strand, the antisense strand, or both strands. For example, an exemplary nucleic acid molecule of the present invention can comprise about 1 to about 5 or more (e.g. about 1, 2, 3, 4, 5, or more) chemically-modified internucleotide linkages having Formula I at the 5′-end of the sense strand, the antisense strand, or both strands. In another non-limiting example, an exemplary nucleic acid molecule of the present invention can comprise one or more (e.g. about 1, 2, 3, 4, 5, 6, 7,. 8, 9, 10, or more) pyrimidine nucleotides with chemically-modified internucleotide linkages having Formula I in the sense strand, the antisense strand, or both strands. In yet another non-limiting example, an exemplary nucleic acid molecule of the present invention can comprise one or more (e.g. about 1, 2, 3, 4, 5, 6, 7, 8, 9, 10, or more) purine nucleotides with chemically-modified internucleotide linkages having Formula I in the sense strand, the antisense strand, or both strands. In another embodiment, a nucleic acid molecule of the present invention having internucleotide linkage(s) of Formula I also comprises a chemically-modified nucleotide or non-nucleotide having any of Formulae I-VII.

In one embodiment, the invention features a chemically-modified short interfering nucleic acid (nucleic acid) molecule capable of mediating RNA interference (RNAi) against a E2F1, NFkB, CREB-1, or CDC2A inside a cell, such cell may be subjected to RNAi in vivo, in vitro, or ex vivo, or in reconstituted in vitro system, wherein the chemical modification comprises one or more (e. g. about 1, 2, 3, 4, 5, 6, 7, 8, 9, 10, or more) nucleotides or non-nucleotides having Formula II: 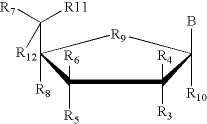
wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base such as adenine, guanine, uracil, cytosine, thymine, aminoadenosine, 5-methylcytosine, 2,6-diaminopurine, or any other non-naturally occurring base that can be complementary or non-complementary to a target RNA or a non-nucleosidic base such as phenyl, naphthyl, 3-nitropyrrole, 5-nitroindole, nebularine, pyridone, pyridinone, or any other non-naturally occurring universal base that can be complementary or non-complementary to target RNA.

The chemically-modified nucleotide or non-nucleotide of Formula II can be present in one or both oligonucleotide strands of the nucleic acid duplex, for example in the sense strand, the antisense strand, or both strands. The nucleic acid molecules of the present invention can comprise one or more chemically-modified nucleotide or non-nucleotide of Formula II at the 3′-end, the 5′-end, or both of the 3′ and 5′ ends of the sense strand, the antisense strand, or both strands. For example, an exemplary nucleic acid molecule of the present invention can comprise about 1 to about 5 or more (e.g. about 1, 2, 3, 4, 5, or more) chemically modified nucleotides or non-nucleotides of Formula II at the 5′-end of the sense strand, the antisense strand, or both strands. In anther non-limiting example, an exemplary nucleic acid molecule of the present invention can comprise about 1 to about 5 or more (e.g. about 1, 2, 3, 4, 5, or more) chemically-modified nucleotides or non-nucleotides of Formula II at the 3′ end of the sense strand, the antisense strand, or both strands.

In one embodiment, the invention features a chemically-modified short interfering nucleic acid (nucleic acid) molecule capable of mediating RNA interference (RNAi) against E2F1, NFkB, CREB-1, or CDC2A inside a cell inside a cell, such cell may be subjected to RNAi in vivo, in vitro, or ex vivo, or in reconstituted in vitro system, wherein the chemical modification comprises one or more (e. g. about 1, 2, 3, 4, 5, 6, 7, 8, 9, 10, or more) nucleotides or non-nucleotides having Formula III: 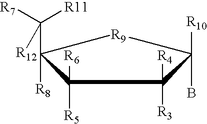
wherein each R3, R4, R5, R6, R7, R8, R10, R 1I and R12 is independently H. OH, alkyl, substituted alkyl, alkaryl or aralkyl, F. Cl, Br, ON, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, 20 O-alkyl-SH, S-alkyl-OH, S-alkyl-SH, alkyl-S-alkyl, alkyl-O-alkyl, ON02, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base such as adenine, guanine, uracil, cytosine, thymine, 2 25 aminoadenosine, 5-methylcytosine, 2,6-diaminopunne, or any other non-naturally occurring base that can be employed to be complementary or non-complementary to target RNA or a non-nucleosidic base such as phenyl, naphthyl, 3-nitropyrrole, 5-nitroindole, nebularine, pyridone, pyridinone, or any other non-naturally occurring universal base that can be complementary or non-complementary to target RNA.

The chemically-modified nucleotide or non-nucleotide of Formula III can be present in one or both oligonucleotide strands of the nucleic acid duplex, for example, in the sense strand, the antisense strand, or both strands. The nucleic acid molecules of the present invention can comprise one or more chemically-modified nucleotide or non-nucleotide of Formula III at the 3′-end, the 5′-end, or both of the 3′ and 5′ ends of the sense strand, the antisense strand, or both strands. For example, an exemplary nucleic acid molecule of the present invention can comprise about 1 to about 5 or more (e.g., about 1, 2, 3, 4, 5, or more) chemically modified nucleotide(s) or non-nucleotide(s) of Formula III at the 5′-end of the sense strand, the antisense strand, or both strands. In another non-limiting example, an exemplary nucleic acid molecule of the present invention can comprise about 1 to about 5 or more (e.g., about 1, 2, 3, 4, 5, or more) chemically-modified nucleotide or non-nucleotide of Formula III at the 3′-end of the sense strand, the antisense strand, or both strands.

In another embodiment, a nucleic acid molecule of the present invention comprises a nucleotide having Formula II or III, wherein the nucleotide having Formula II or III is in an inverted configuration. For example, the nucleotide having Formula II or III is connected to the nucleic acid construct in a 3′-3′, 3′-2′, 2′-3′, or 5′-5′ configuration, such as at the 3′-end, the 5′-end, or both of the 3′ and 5′-ends of one or both nucleic acid strands.

In one embodiment, the invention features a chemically-modified short interfering nucleic acid (nucleic acid) molecule capable of mediating RNA interference (RNAi) against a E2F1, NFkB, CREB-1, or CDC2A inside a cell, such cell may be subjected to RNAi in vivo, in vitro, or ex vivo, or in reconstituted in vitro system, wherein the chemical modification comprises a 5′-tenninal phosphate group having Formula IV: 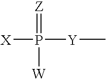
wherein each X and Y is independently O, S, N, alkyl, substituted alkyl, or alkylhalo, wherein each Z and W is independently O, S, N, alkyl, substituted alkyl, O-alkyl, S alkyl, alkaryl, aralkyl, or alkylhalo; and wherein W. X, Y and Z are not all O. In one embodiment, the invention features a nucleic acid molecule having a 5′-terminal 5 phosphate group having Formula IV on the target-complementary strand, for example, a strand complementary to a target RNA, wherein the nucleic acid molecule comprises an all RNA nucleic acid molecule. In another embodiment, the invention features a nucleic acid molecule having a 5′-terminal phosphate group having Formula IV on the target-complementary strand wherein the nucleic acid molecule also comprises about 1 to about 3 (e.g. about 1, 2, or 3) nucleotide 3′-terminal nucleotide overhangs having about 1 to about 4 (e.g. about 1, 2, 3, or 4) deoxyribonucleotides on the 3′-end of one or both strands. In another embodiment, a 5′-tenninal phosphate group having Formula IV is present on the target complementary strand of a nucleic acid molecule of the present invention, for example a nucleic acid molecule having chemical modifications having any of Formulae I-VII.

In one embodiment, the invention features a chemically-modified short interfering nucleic acid (nucleic acid) molecule capable of mediating RNA interference (RNAi) against E2F1, NFkB, CREB-1, or CDC2A inside a cell, such cell may be subjected to RNAi in vivo, in vitro, or ex vivo, or in reconstituted in vitro system, wherein the chemical modification comprises one or more phosphorothioate internucleotide linkages. For example, in a non-limiting example, the invention features a chemically-modified short interfering nucleic acid (nucleic acid) having about 1, 2, 3, 4, 5, 6, 7, 8 or more phosphorothioate internucleotide linkages in one nucleic acid strand. In yet another embodiment, the invention features a chemically-modified short interfering nucleic acid (nucleic acid) individually having about 1, 2, 3, 4, 5, 6, 7, 8 or more phosphorothioate internucleotide linkages in both nucleic acid strands. The phosphorothioate internucleotide linkages can be present in one or both oligonucleotide strands of the nucleic acid duplex, for example in the sense strand, the antisense strand, or both strands. The nucleic acid molecules of the present invention can comprise one or more phosphorothioate internucleotide linkages at the 3′-end, the 5′-end, or both of the 3′- and 5′-ends of the sense strand, the antisense strand, or both strands. For example, an exemplary nucleic acid molecule of the present invention can comprise about 1 to about 5 or more (e.g. about 1, 2, 3, 4, 5, or more) consecutive phosphorothioate internucleotide linkages at the 5′-end of the sense strand, the antisense strand, or both strands. In another non-limiting example, an exemplary nucleic acid molecule of the present invention can comprise one or more (e.g. about 1, 2, 3, 4, 5, 6, 7, 8, 9, 10, or more) pyrimidine phosphorothioate internucleotide linkages in the sense strand, the antisense strand, or both strands. In yet another non-limiting example, an exemplary nucleic acid molecule of the present invention can comprise one or more (e.g. about 1, 2, 3, 4, 5, 6, 7, 8, 9, 10, or more) purine phosphorothioate 5 internucleotide linkages in the sense strand, the antisense strand, or both strands.

In one embodiment, the invention features a nucleic acid molecule, wherein the sense strand comprises one or more, for example, about 1, 2, 3, 4, 5, 6, 7, 8, 9, 10, or more phosphorothioate internucleotide linkages, and/or one or more (e.g. about 1, 2, 3, 4, 5, 6, 7, 8, 9, 10 or more) 2′-deoxy, 2′-O-methyl, 2′-deoxy-2′-fluoro, and/or about one or more (e.g. about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and wherein the antisense strand comprises about 1 to about 10 or more, specifically about 1, 2, 3, 4, 5, 6, 7, 8, 9, 10, or more phosphorothioate internucleotide linkages, and/or one or more (e.g., about 1, 2, 3, 4, 5, 6, 7, 8, 9, 10 or 15 more) 2′-deoxy, 2′-O-methyl, 2′-deoxy-2′-fluoro, and/or one or more (e.g. about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In another embodiment, one or more, for example about 1, 2, 3, 4, 5, 6, 7, 8, 9, 10, or more, pyrimidine nucleotides of the sense and/or antisense nucleic acid strand are chemically-modified with 2′-deoxy, 2′-O-methyl and/or 2′-deoxy-2′-fluoro nucleotides, with or without one or more, for example about 1, 2, 3, 4, 5, 6, 7, 8, 9, 10, or more, phosphorothioate internucleotide linkages and/or a terminal cap molecule at the 3′-end, the 5′-end, or both of the 3′- and 5′-ends, being present in the same or different strand.

In another embodiment, the invention features a nucleic acid molecule, wherein the sense strand comprises about 1 to about 5, specifically about 1, 2, 3, 4, or 5 phosphorothioate internucleotide linkages, and/or one or more (e.g. about 1, 2, 3, 4, 5, or more) 2′-deoxy, 2′-O-methyl, 2′-deoxy-2′-fluoro, and/or one or more (e.g. about 1, 2, 3, 4, 5, or more) universal base modified nucleotides, and optionally a terminal cap molecule at the 3-end, the 5′-end, or both of the 3′- and 5′-ends of the sense strand; and wherein the antisense strand comprises about 1 to about 5 or more, specifically about 1, 2, 3, 4, 5, or more phosphorothioate internucleotide linkages, and/or one or more (e.g. about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In another embodiment, one or more, for example about 1, 2, 3, 4, 5, 6, 7, 8, 9, 10, or more, pyrimidine nucleotides of the sense and/or antisense nucleic acid strand are chemically-modified with 2′-deoxy, 2′-O-methyl and/or 2′-deoxy-2′-fluoro nucleotides, with or without about 1 to about 5 or more, for example about 1, 2, 3, 4, 5, or more phosphorothioate internucleotide linkages and/or a terminal cap molecule at the 3′-end, the 5′-end, or both of the 3′- and 5′-ends, being present in the same or different strand. In one embodiment, the invention features a nucleic acid molecule, wherein the antisense strand comprises one or more, for example, about 1, 2, 3, 4, 5, 6, 7, 8, 9, 10, or more phosphorothioate internucleotide linkages, and/or about one or more (e.g. about 1, 2, 3, 4, 5, 6, 7, 8, 9, or more) 2′-deoxy, 2′-O-methyl, 2′-deoxy-2′-fluoro, and/or one or more (e.g. about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and wherein the antisense strand comprises about 1 to about 10 or more, specifically about 1, 2, 3, 4, 5, 6, 7, 8, 9, 10 or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g. about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In another embodiment, one or more, for example about 1, 2, 3, 4, 5, 6, 7, 8, 9, 10 or more pyrimidine nucleotides of the sense and/or antisense nucleic acid strand are chemically-modified with 2′-deoxy, 2′-O-methyl and/or 2′-deoxy-2′-fluoro nucleotides, 25 with or without one or more, for example, about 1, 2, 3, 4, 5, 6, 7, 8, 9, 10 or more phosphorothioate internucleotide linkages and/or a terminal cap molecule at the 3′-end, the 5′-end, or both of the 3′ and 5′-ends, being present in the same or different strand.

In another embodiment, the invention features a nucleic acid molecule, wherein the antisense strand comprises about 1 to about 5 or more, specifically about 1, 2, 3, 4, 5 or more phosphorothioate internucleotide linkages, and/or one or more (e.g. about 1, 2, 3, 4, 5, 6, 7, 8, 9, 10 or more) 2′-deoxy, 2′-O-methyl, 2′-deoxy-2′-fluoro, and/or one or more (e.g. about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and wherein the antisense strand comprises about 1 to about 5 or more, specifically about 1, 2, 3, 4, 5 or more phosphorothioate internucleotide linkages, and/or one or more (e.g. about 1, 2, 3, 4, 5, 6, 7, 8, 9, 10 or more) 2′-deoxy, 2′-O-methyl, 5 2′-deoxy-2′-fluoro, and/or one or more (e.g. about 1, 2, 3, 4, 5, 6, 7, 8, 9, or more) universal base modified nucleotides, and optionally a terminal cap molecule at the 3′ end, the 5′-end, or both of the 3′- and 5′-ends of the antisense strand. In another embodiment, one or more, for example about 1, 2, 3, 4, 5, 6, 7, 8, 9, 10 or more pyrimidine nucleotides of the sense and/or antisense nucleic acid strand are chemically modified with 2′-deoxy, 2′-O-methyl and/or 2′-deoxy-2′-fluoro nucleotides, with or without about 1 to about 5, for example about 1, 2, 3, 4, 5 or more phosphorothioate internucleotide linkages and/or a terminal cap molecule at the 3′-end, the 5′-end, or both of the 3′- and 5′-ends, being present in the same or different strand.

In one embodiment, the invention features a chemically-modified short interfering nucleic acid (siNA) molecule having about 1 to about 5, specifically about 1, 2, 3, 4, 5 or more phosphorothioate internucleotide linkages in each strand of the siNA molecule. In another embodiment, the invention features a siNA molecule comprising 2′-5′ internucleotide linkages. The 2′-5′ internucleotide linkage(s) can be at the 3′-end, the 5′ end, or both of the 3′- and 5′-ends of one or both siNA sequence strands. In addition, the 2′-5′ internucleotide linkage(s) can be present at various other positions within one or both siNA sequence strands, for example, about 1, 2, 3, 4, 5, 6, 7, 8, 9, 10, or more including every internucleotide linkage of a pyrimidine nucleotide in one or both strands of the siNA molecule can comprise a 2′-5′ internucleotide linkage, or about 1, 2, 3, 4, 5, 6, 7, 8, 9, 10, or more including every internucleotide linkage of a purine nucleotide in 25 one or both strands of the siNA molecule can comprise a 2′-5′ internucleotide linkage.

In another embodiment, a chemically-modified siNA molecule of the present invention comprises a duplex having two strands, one or both of which can be chemically modified, wherein each strand is about 18 to about 27 (e.g. about 18, 19, 20, 21, 22, 23, 24, 25, 26, or 27) nucleotides in length, wherein the duplex has about 18 to about 23 (e.g. about 18, 19, 20, 21, 22, or 23) base pairs, and wherein the chemical modification comprises a structure having any of Formulae I-VII. For example, an exemplary chemically-modified nucleic acids molecule of the present invention comprises a duplex having two strands, one or both of which can be chemically-modified with a chemical modification having any of Formulae I-VII or any combination thereof, wherein each strand consists of about 21 nucleotides, each having a 2-nucleotide 3′-terminal nucleotide overhang, and 5 wherein the duplex has about 19 base pairs. In another embodiment, a nucleic acid molecule of the present invention comprises a single stranded hairpin structure, wherein the nucleic acid is about 36 to about 70 (e.g., about 36, 40, 45, 50, 55, 60, 65, or 70) nucleotides in length having about 18 to about 23 (e.g., about 18, 19, 20, 21, 22, or 23) base pairs, and wherein the nucleic acid can include a chemical modification comprising a structure having any of Formulae I-VII or any combination thereof. For example, an exemplary chemically modified nucleic acid molecule of the present invention comprises a linear oligonucleotide having about 42 to about 50 (e.g. about 42, 43, 44, 45, 46, 47, 48, 49, or 50) nucleotides that is chemically-modified with a chemical modification having any of Formulae I-VII or any combination thereof, wherein the linear oligonucleotide forms a hairpin structure having about 19 base pairs and a 2-nucleotide 3′-terminal nucleotide overhang. In another embodiment, a linear hairpin nucleic acid molecule of the present invention contains a stem loop motif, wherein the loop portion of the nucleic acid molecule is biodegradable. For example, a linear hairpin nucleic acid molecule of the present invention is designed such that degradation of the loop portion of the nucleic acid molecule in vivo can generate a double-stranded nucleic acid molecule with 3′-terminal overhangs, such as 3′-terminal-nucleotide overhangs comprising about 2 nucleotides. In another embodiment, a nucleic acid molecule of the present invention comprises a circular nucleic acid molecule, wherein the nucleic acid is about 38 to about 70 (e.g., about 38, 40, 45, 50, 55, 60, 65, or 70) nucleotides in length having about 18 to about 23 (e.g. about 18, 19, 20, 21, 22, or 23) base pairs, and wherein the nucleic acid can include a chemical modification, which comprises a structure having any of Formulae I-VII or any combination thereof. For example, an exemplary chemically-modified nucleic acid molecule of the invention comprises a circular oligonucleotide having about 42 to about 50 (e.g. about 42, 43, 44, 45, 46, 47, 48, 49, or 50) nucleotides that is chemically-modified with a chemical modification having any of Formulae I-VII or any combination thereof, wherein the circular oligonucleotide forms a dumbbell shaped structure having about 19 base pairs and 2 loops.

In another embodiment, a circular nucleic acid molecule of the present invention contains two loop motifs, wherein one or both loop portions of the nucleic acid molecule is biodegradable. For example, a circular nucleic acid molecule of the present invention is designed such that degradation of the loop portions of the nucleic acid molecule in viva can generate a double-stranded nucleic acid molecule with 3′-terminal overhangs, such as 3′-terminal nucleotide overhangs comprising about 2 nucleotides.

In one embodiment, a nucleic acid molecule of the present invention comprises at least one (e.g. about 1, 2, 3, 4, 5, 6, 7, 8, 9, 10, or more) abasic moiety, for example a compound having Formula V: 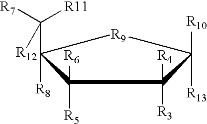
wherein each R3, R4, R5, R6, R7, R8, R10, R11, R12, and R13 is independently H, OH, alkyl, substituted alkyl, alkaryl or aralkyl, F. Cl, Br, ON, CF3, OCF3, OCN, O-alkyl, S alkyl, N-alkyl, O-alkenyl, S-alkenyl, N-alkenyl, SO-alkyl, alkyl-OSH, alkyl-OH, O alkyl-OH, O-alkyl-SH, S-alkyl-OH, S-alkyl-SH, alkyl-S-alkyl, alkyl-O-alkyl, ONO2, 15 NO2, N3, NH2, aminoalkyl, aminoacid, aminoacyl, ONH2, O-aminoalkyl, O-aminoacid, O-amino acyl, hetero cyclo alkyl, hetero cyclo alkaryl, amino alkylamino, polyalklylamino, substituted silyl, or group having Formula I; R9 is O, S, CH2, S═O, CHF, or CF2.

In one embodiment, a nucleic acid molecule of the present invention comprises at least one (e.g. about 1, 2, 3, 4, 5, 6, 7, 8, 9, 10, or more) inverted abasic moiety, for example a compound having Formula VI: 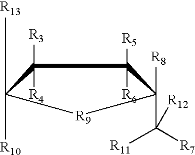
wherein each R3, R4, R5, R6, R7, R8, R10, R11, R12, and R13 is independently H, OH, alkyl, substituted alkyl, alkaryl or aralkyl, F. Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and either R2, R3, R8 or R13 serve as points of attachment to the nucleic acid molecule of the invention.

In another embodiment, a nucleic acid molecule of the present invention comprises at least one (e.g. about 1, 2, 3, 4, 5, 6, 7, 8, 9, 10, or more) substituted polyalkyl moieties, for example a compound having Formula VII: 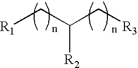
wherein each “n” is independently an integer from 1 to 12, each R1, R2 and R3 is independently H. OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O 20 aminoalkyl, O-aminoacid, O-aminoacyl, heterocycloalkyl, heterocycloalkaryl, aminoalkylamino, polyalklylamino, substituted silyl, or a group having Formula I, and R1, R2 or R3 serves as points of attachment to the nucleic acid molecule of the present invention.

In another embodiment, the invention features a compound having Formula VII, wherein R1 and R2 are hydroxyl (OH) groups, n=1, and R3 comprises O and is the point of attachment to the 3′-end, the 5′-end, or both of the 3′ and 5′-ends of one or both strands of a double-stranded nucleic acid molecule of the present invention or to a single-stranded nucleic acid molecule of the present invention. This modification is referred to herein as “glyceryl”.

In another embodiment, a moiety having any of Formula V, VI or VII of the present invention is at the 3′-end, the 5′-end, or both of the 3′ and 5′-ends of a nucleic acid molecule of the present invention. For example, a moiety having Formula V, VI or VII can be present at the 10 3′-end, the 5′-end, or both of the 3′ and 5′-ends of the antisense strand, the sense strand, or both antisense and sense strands of the nucleic acid molecule. In addition, a moiety having Formula VII can be present at the 3′-end or the 5′-end of a hairpin nucleic acid molecule as described herein.

In another embodiment, a nucleic acid molecule of the present invention comprises an abasic residue having Formula V or VI, wherein the abasic residue having Formula VI or VI is connected to the nucleic acid construct in a 3′-3′, 3′-2′, 2′-3′, or 5′-5′ configuration, such as at the 3′-end, the 5′-end, or both of the 3′ and 5′-ends of one or both nucleic acid strands.

In one embodiment, a nucleic acid molecule of the present invention comprises one or more (e.g., about 1, 2, 3, 4, 5, 6, 7, 8, 9, 10, or more) locked nucleic acid (LNA) nucleotides, for example at the 5′-end, the 3′-end, both of the 5′ and 3′-ends, or any combination thereof, of the nucleic acid molecule.

In another embodiment, a nucleic acid molecule of the present invention comprises one or more (e.g. about 1, 2, 3, 4, 5, 6, 7, 8, 9, 10, or more) acyclic nucleotides, for example at the 5′-end, the 3′-end, both of the 5′ and 3′ends, or any combination thereof, of the nucleic acid molecule.

In one embodiment, the invention features a chemically-modified short interfering nucleic acid (nucleic acid) molecule of the present invention, wherein the chemically-modified nucleic acid comprises a sense region, where any (e.g., one or more or all) pyrimidine nucleotides present in the sense region are 2′-deoxy-2′-fluoro pyrimidine nucleotides (e.g. wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where any (e.g. one or more or all) purine nucleotides present in the sense region are 2′-deoxy purine nucleotides (e.g. wherein all purine nucleotides are 2′-deoxy purine nucleotides or alternately a plurality of purine nucleotides are 2′-deoxy purine nucleotides).

In one embodiment, the invention features a chemically-modified short interfering nucleic acid (nucleic acid) molecule of the present invention, wherein the chemically-modified nucleic acid comprises a sense region, where any (e.g. one or more or all) pyrimidine nucleotides present in the sense region are 2′-deoxy-2′-fluoro pyrimidine nucleotides (e.g. wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where any (e.g. one or more or all) purine nucleotides present in the sense region are 2′-deoxy purine nucleotides (e.g. wherein all purine nucleotides are 2′-deoxy purine nucleotides or alternately a plurality of purine nucleotides are 2′-deoxy purine nucleotides), wherein any nucleotides comprising a 3′-terminal nucleotide overhang that are present in said sense region are 2′-deoxy nucleotides.

In one embodiment, the invention features a chemically-modified short interfering nucleic acid (nucleic acid) molecule of the present invention, wherein the chemically-modified nucleic acid comprises an antisense region, where any (e.g. one or more or all) pyrimidine nucleotides present in the antisense region are 2′-deoxy-2′-fluoro pyrimidine nucleotides (e.g. wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any (e.g. one or more or all) purine nucleotides present in the antisense region are 2′-O-methyl purine nucleotides (e.g. wherein all purine nucleotides are 2′-O-methyl purine nucleotides or alternately a plurality of purine nucleotides are 2′-O-methyl purine nucleotides).

In one embodiment, the invention features a chemically-modified short interfering nucleic acid (nucleic acid) molecule of the present invention, wherein the chemically-modified nucleic acid comprises an antisense region, where any (e.g. one or more or all) pyrimidine nucleotides present in the antisense region are 2′-deoxy-2′-fluoro pyrimidine nucleotides (e.g. wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any (e.g., one or more or all) purine nucleotides present in the antisense region are 2′-O-methyl purine nucleotides (e.g., wherein all purine nucleotides are 2′-O-methyl purine nucleotides or alternately a plurality of purine nucleotides are 2′ 5 O-methyl purine nucleotides), wherein any nucleotides comprising a 3′-terminal nucleotide overhang that are present in said antisense region are 2′-deoxy nucleotides.

In one embodiment, the invention features a chemically-modified short interfering nucleic acid (nucleic acid) molecule of the present invention, wherein the chemically-modified nucleic acid comprises an antisense region, where any (e.g. one or more or all) pyrimidine nucleotides present in the antisense region are 2′-deoxy-2′-fluoro pyrimidine nucleotides (e.g. wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where any (e.g., one or more or all) purine nucleotides present in the antisense region are 2′-deoxy purine nucleotides (e.g. wherein all purine nucleotides are 2′-deoxy purine nucleotides or alternately a plurality of purine nucleotides are 2′-deoxy purine nucleotides).

In one embodiment, the invention features a chemically-modified short interfering nucleic acid (nucleic acid) molecule of the present invention capable of mediating RNA interference (RNAi) against a E2F1, NFkB, CREB-1, or CDC2A inside a cell, such cell may be subjected to RNAi in vivo, in vitro, or ex vivo, or in reconstituted in vitro system, wherein the chemically-modified nucleic acids comprises a sense region, where one or more pyrimidine nucleotides present in the sense region are 2′-deoxy-2′-fluoro pyrimidine nucleotides (e.g. wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where one or more purine nucleotides present in the sense region are 2′ 25 deoxy purine nucleotides (e.g. wherein all purine nucleotides are 2′-deoxy purine nucleotides or alternately a plurality of purine nucleotides are 2′-deoxy purine nucleotides), and inverted deoxy abasic modifications that are optionally present at the 3′-end, the 5′-end, or both of the 3′ and 5′-ends of the sense region, the sense region optionally further comprising a 3′-terminal overhang having about 1 to about 4 (e.g., about 1, 2, 3, or 4) 2′-deoxyribonucleotides; and wherein the chemically-modified short interfering nucleic acid molecule comprises an antisense region, where one or more pyrimidine nucleotides present in the antisense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein one or more purine nucleotides present in the antisense region are 2′-O-methyl purine nucleotides (e.g., wherein all purine nucleotides 5 are 2′-O-methyl purine nucleotides or alternately a plurality of purine nucleotides are 2′-O-methyl purine nucleotides), and a terminal cap modification, such as any modification described herein, that is optionally present at the 3′-end, the 5′-end, or both of the 3′ and 5′-ends of the antisense sequence, the antisense region optionally further comprising a 3′-terminal nucleotide overhang having about 1 to about 4 (e.g., about 1, 2, 3, or 4) 2′-deoxynucleotides, wherein the overhang nucleotides can further comprise one or more (e.g., 1, 2, 3, or 4) phosphorothioate internucleotide linkages.

In one embodiment, the invention features a chemically-modified short interfering nucleic acid (nucleic acid) molecule of the present invention capable of mediating RNA interference (RNAi) against E2F1, NFkB, CREB-1, or CDC2A inside a cell, such cell may be subjected to RNAi in vivo, in vitro, or ex vivo, or in reconstituted in vitro system, wherein the nucleic acid comprises a sense region, where one or more pyrimidine nucleotides present in the sense region are 2′-deoxy-2′-fluoro pyrimidine nucleotides (e.g., wherein all pyrimidine nucleotides are 2′-deoxy-2′-fluoro pyrimidine nucleotides or alternately a plurality of 20 pyrimidine nucleotides are 2′-deoxy-2′-fluoro pyrimidine nucleotides), and where one or more purine nucleotides present in the sense region are purine ribonucleotides (e.g., wherein all purine nucleotides are purine ribonucleotides or alternately a plurality of purine nucleotides are purine ribonucleotides), and inverted deoxy abasic modifications that are optionally present at the 3′-end, the 5′-end, or both of the 3′ and 5′-ends of the sense region, the sense region optionally further comprising a 3′-terminal overhang having about 1 to about 4 (e.g., about 1, 2, 3, or 4) 2′-deoxyribonucleotides; and wherein the nucleic acid comprises an antisense region, where one or more pyrimidine nucleotides present in the antisense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides), and a terminal cap modification, such as any modification described herein, that is optionally present at the 3′-end, the 5′-end, or both of the 3′ and 5′-ends of the antisense sequence, the antisense region optionally further comprising a 3′-terminal nucleotide overhang having about 1 to about 4 (e.g., about 1, 2, 3, or 4) 2′-deoxynucleotides, wherein the overhang nucleotides can further comprise one or more (e.g., 1, 2, 3, or 4) phosphorothioate internucleotide linkages.

In one embodiment, the invention features a chemically-modified short interfering nucleic acid (nucleic acid) molecule of the present invention capable of mediating RNA interference (RNAi) against E2F1, NFkB, CREB-1, or CDC2A inside a cell, such cell may be subjected to RNAi in vivo, in vitro, or ex vivo, or in reconstituted in vitro system, wherein the chemically-modified nucleic acids comprises a sense region, where one or more pyrimidine nucleotides present in the sense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and for example where one or more purine nucleotides present in the sense region are selected from the group consisting of 2′-deoxy nucleotides, locked nucleic acid (LNA) nucleotides, 2′-methoxyethyl nucleotides, 4′-thionucleotides, and 2′-O-methyl nucleotides (e.g. wherein all purine nucleotides are selected from the group consisting of 2′-deoxy nucleotides, locked nucleic acid (LNA) nucleotides, 2′-methoxyethyl nucleotides, 4′-thionucleotides, and 2′-O-methyl nucleotides or alternately a plurality of purine nucleotides are selected from the group consisting of 2′-deoxy nucleotides, locked nucleic acid (LNA) nucleotides, 2′-methoxyethyl nucleotides, 4′-thionucleotides, and 2′-O-methyl nucleotides), and wherein inverted deoxy abasic modifications are optionally present at the 3′-end, the 5′-end, or both of the 3′ and 5′-ends of the sense region, the sense region optionally further comprising a 3′-terminal overhang having about 1 to about 4 (e.g., about 1, 2, 3, or 4) 2′-deoxyribonucleotides, and wherein the chemically-modified short interfering nucleic acid molecule comprises an antisense region, where one or more pyrimidine nucleotides present in the antisense region are 2′ deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein one or more purine nucleotides present in the antisense region are selected from the group consisting of 2′-deoxy nucleotides, locked nucleic acid (LNA) nucleotides, 2′-methoxyethyl nucleotides, 4′-thionucleotides, and 2′-0-methyl nucleotides (e.g., wherein all purine nucleotides are selected from the group consisting of 2′-deoxy nucleotides, locked nucleic acid (LNA) nucleotides, 2′-methoxyethyl nucleotides, 4′-thionucleotides, and 2′ 5 O-methyl nucleotides or alternately a plurality of purine nucleotides are selected from the group consisting of 2′-deoxy nucleotides, locked nucleic acid (LNA) nucleotides, 2′-methoxyethyl nucleotides, 4′-thionucleotides, and 2′-0-methyl nucleotides), and a terminal cap modification, such as any modification described herein, that is optionally present at the 3′-end, the 5′-end, or both of the 3′ and 5′-ends of the antisense sequence, the antisense region optionally farther comprising a 3′-terminal nucleotide overhang having about 1 to about 4 (e.g., about 1, 2, 3, or 4) 2′-deoxynucleotides, wherein the overhang nucleotides can further comprise one or more (e.g., 1, 2, 3, or 4) phosphorothioate internucleotide linkages.

In another embodiment, any modified nucleotides present in the nucleic acid molecules of the invention, preferably in the antisense strand of the nucleic acid molecules of the invention, but also optionally in the sense and/or both antisense and sense strands, comprise modified nucleotides having properties or characteristics similar to naturally occurring ribonucleotides. For example, the invention features nucleic acid molecules including modified nucleotides having a Northern conformation (e.g., Northern pseudorotation cycle, see for example Saenger, Principles of Nucleic Acid Structure, Springer-Verlag ea., 1984). As such, chemically modified nucleotides present in the nucleic acid molecules of the present invention, preferably in the antisense strand of the nucleic acid molecules of the present invention, but also optionally in the sense and/or both antisense and sense strands, are resistant to nuclease degradation while at the same time maintaining the capacity to mediate RNAi. Non limiting examples of nucleotides having a northern configuration include locked nucleic acid (LNA) nucleotides (e.g., 2′-0, 4′-C-methylene-(D-ribofuranosyl) nucleotides); 2′-methoxyethoxy (MOE) nucleotides; 2′-methyl-thio-ethyl, 2′-deoxy-2′-fluoro nucleotides, 2′-deoxy-2′-chloro nucleotides, 2′-azido nucleotides, and 2′-0-methyl nucleotides. In one embodiment, the invention features a chemically-modified short interfering nucleic acid molecule (nucleic acid) capable of mediating RNA interference (RNAi) against a E2F1, NFkB, CREB-1, or CDC2A inside a cell, such cell may be subjected to RNAi in vivo, in vitro, or ex vivo, or in reconstituted in vitro system, wherein the chemical modification comprises a conjugate covalently attached to the chemically-modified nucleic acids molecule. In another embodiment, the conjugate is covalently attached to the chemically-modified nucleic acids molecule via a biodegradable linker. In one embodiment, the conjugate molecule is attached at the 3′-end of either the sense strand, the antisense strand, or both strands of the chemically-modified nucleic acids molecule. In another embodiment, the conjugate molecule is attached at the 5′ or 3′ end of either the sense strand, the antisense strand, or both strands of the chemically-modified nucleic acids molecule. In yet another embodiment, the conjugate molecule is attached both the 3′end and 5′-end of either the sense strand, the antisense strand, or both strands of the chemically-modified nucleic acids molecule, or any combination thereof. In one embodiment, a conjugate molecule of the present invention comprises a molecule that facilitates delivery of a chemically-modified nucleic acids molecule into a biological system, such as a cell. In another embodiment, the conjugate molecule attached to the chemically-modified nucleic acids molecule is a poly ethylene glycol, human serum albumin, or a ligand for a cellular receptor that can mediate cellular uptake.

Examples of specific conjugate molecules contemplated by the instant invention that can be attached to chemically-modified nucleic acids molecules are described in Vargeese et al., U.S. Ser. No. 10/201,394, incorporated by reference herein. The type of conjugates used and the extent of conjugation of nucleic acid molecules of the invention can be evaluated for improved pharmacokinetic profiles, bioavailability, and/or stability of nucleic acid constructs while at the same time maintaining the ability of the nucleic acid to mediate RNAi activity. As such, one skilled in the art can screen nucleic acid constructs that are modified with various conjugates to determine whether the nucleic acid conjugate complex possesses improved properties while maintaining the ability to mediate RNAi, for example in animal models as are generally known in the art.

In one embodiment, the invention features a short interfering nucleic acid (nucleic acid) molecule of the present invention, wherein the nucleic acid further comprises a nucleotide, non nucleotide, or mixed nucleotide/non-nucleotide linker that joins the sense region of the nucleic acid to the antisense region of the nucleic acid. In one embodiment, a nucleotide linker of the present invention can be a linker of 2 nucleotides in length, for example 3, 4, 5, 6, 7, 8, 9, or 10 nucleotides in length. In another embodiment, the nucleotide linker can be a nucleic acid aptamer. By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art. (See, for example, Gold et al., 1995, Annul Rev. Biochem., 64, 763; Brody and Gold, 2000, J. Biotechnol., 74, 5; Sun, 2000, Curr. Opin. Mol. Ther., 2, 100; Fusser, 2000, J; Biotechnol., 74, 27; Hermann and Patel, 2000, Science, 287, 820; and Jayasena, 1999, Clinical Chemistry, 45, 1628.) In yet another embodiment, a non-nucleotide linker of the present invention comprises abasic nucleotide, polyether, polyamine, polyamide, peptide, carbohydrate, lipid, polyhydrocarbon, or other polymeric compounds (e.g. polyethylene glycols such as those having between 2 and 100 ethylene glycol units). Specific examples include those described by Seela and Kaiser, Nucleic Acids Res. 1990, 18:6353 and Nucleic Acids Res. 1987, 15:3113; Cload and Schepartz, J. Am. Chem. Soc. 1991,113:6324; Richardson and Schepartz, J; Am. Sheen. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., 20 Tetrahedron Lett. 1993, 34:301; Ono et al., Biochemistry 1991, 30:9914; An1old et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine, for example at the C-1 position of the sugar.

In one embodiment, the invention features a short interfering nucleic acid (nucleic acid) molecule capable of mediating RNA interference (RNAi) inside a cell, such cell may be subjected to RNAi in vivo, in vitro, or ex vivo, or in reconstituted in vitro system, wherein one or both strands of the nucleic acid molecule that are assembled from two separate oligonucleotides do not comprise any ribonucleotides. All positions within the nucleic acid can include chemically modified nucleotides and/or non-nucleotides such as nucleotides and or non-nucleotides having Formula I, II, III, IV, V, VI, or VII or any combination thereof to the extent that the ability of the nucleic acid molecule to support RNAi activity in a cell is maintained.

In one embodiment, a nucleic acid molecule of the present invention is a single stranded nucleic acid molecule that mediates RNAi activity in a cell or reconstituted in vitro system, wherein the nucleic acid molecule comprises a single stranded polynucleotide having complementarily to a target nucleic acid sequence. In another embodiment, the single stranded nucleic acid molecule of the present invention comprises a 5′-tenninal phosphate group. In another embodiment, the single stranded nucleic acid molecule of the present invention comprises a 5′-terminal phosphate group and a 3′-terminal phosphate group (e.g., a 2′,3′-cyclic phosphate). In another embodiment, the single stranded nucleic acid molecule of the present invention comprises about 19 to about 29 nucleotides. In yet another embodiment, the single stranded nucleic acid molecule of the present invention comprises one or more chemically modified nucleotides or non-nucleotides described herein.

For example, all the positions within the nucleic acid molecule can include chemically-modified nucleotides such as nucleotides having any of Formulae I-VII, or any combination thereof to the extent that the ability of the nucleic acid molecule to support RNAi activity in a cell is maintained.

In one embodiment, a nucleic acid molecule of the present invention is a single stranded nucleic acid molecule that mediates RNAi activity in a cell, such cell may be subjected to RNAi in vivo, in vitro, or ex vivo, or in reconstituted in vitro system, wherein the nucleic acid molecule comprises a single stranded polynucleotide having complementarily to a target nucleic acid sequence, and wherein one or more pyrimidine nucleotides present in the nucleic acid are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides), and a terminal cap modification, such as any modification described herein, that is optionally present at the 3′-end, the 5′-end, or both of the 3′ and 5′-ends of the antisense sequence, the nucleic acid optionally further comprising about 1 to about 4 (e.g., about 1, 2, 3, or 4) terminal 2′-deoxynucleotides at tile 3′-end of the nucleic acid molecule, wherein the terminal nucleotides can further comprise one or more (e.g., 1, 2, 3, or 4) phosphorothioate internucleotide linkages, and wherein the nucleic acid optionally further comprises a terminal phosphate group, such as a 5′-terminal phosphate group.

In one embodiment, a nucleic acid molecule of the present invention is a single stranded-nucleic acid molecule that mediates RNAi activity in a cell, such cell may be subjected to RNAi in vivo, in vitro, or ex vivo, or in reconstituted in vitro system, wherein the nucleic acid molecule comprises a single stranded polynucleotide having complementarily to a target nucleic acid sequence, and wherein one or more pyrimidine nucleotides present in the nucleic acid are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any purine nucleotides present in the antisense region are 2′-deoxy purine nucleotides (e.g., wherein all purine nucleotides are 2′-deoxy purine nucleotides or alternately a plurality of purine nucleotides are 2′-deoxy purine nucleotides), and a terminal cap modification, such as any modification described herein, that is optionally present at the 3′-end, the 5′-end, or both of the 3′ and 5′-ends of the antisense sequence, the nucleic acid optionally further comprising about 1 to about 4 (e.g., about 1, 2, 3, or 4) terminal 2′-deoxynucleotides at the 3′-end of the nucleic acid molecule, wherein the terminal nucleotides can further comprise one or more (e.g., 1, 2, 3, or 4) phosphorothioate internucleotide linkages, and wherein the nucleic acid optionally further comprises a terminal phosphate group, such as a 5′-terminal phosphate group.

In one embodiment, a nucleic acid molecule of the present invention is a single stranded nucleic acid molecule that mediates RNAi activity in a cell or reconstituted in vitro system, wherein the nucleic acid molecule comprises a single stranded polynucleotide having complementarily to a target nucleic acid sequence, and wherein one or more pyrimidine nucleotides present in the nucleic acid are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any purine nucleotides present in the antisense region are locked nucleic acid (LNA) nucleotides (e.g., wherein all purine nucleotides are LNA nucleotides or alternately a plurality of purine nucleotides are LNA nucleotides), and a terminal cap modification, such as any modification described herein, that is optionally present at the 3′-end, the 5′-end, or both of the 3′ and 5′ends of the antisense sequence, the nucleic acid optionally further comprising about 1 to about 4 (e.g., about 1, 2, 3, or 4) terminal 2′-deoxynucleotides at the 3′-end of the nucleic acid molecule, wherein the terminal nucleotides can further comprise one or more (e.g., 1, 2, 3, or 4) 5 phosphorothioate internucleotide linkages, and wherein the nucleic acid optionally further comprises a terminal phosphate group, such as a 5′-terminal phosphate group.

In one embodiment, a nucleic acid molecule of the present invention is a single stranded nucleic acid molecule that mediates RNAi activity in a cell, such cell may be subjected to RNAi in vivo, in vitro, or ex vivo, or in reconstituted in vitro system, wherein the nucleic acid molecule comprises a single stranded polynucleotide having complementarily to a target nucleic acid sequence, and wherein one or more pyrimidine nucleotides present in the nucleic acid are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any purine nucleotides present in the antisense region are 2′-methoxyethyl purine nucleotides (e.g., wherein all purine nucleotides are 2′-methoxyethyl purine nucleotides or alternately a plurality of purine nucleotides are 2′-methoxyethyl phone nucleotides), and a terminal cap modification, such as any modification described herein, that is optionally present at the 3′-end, the 5′-end, or both of the 3′ and 5′-ends of the antisense sequence, the nucleic acid optionally further comprising about 1 to about 4 (e.g., about 1, 2, 3, or 4) terminal 2′-deoxynucleotides at the 3′-end of the nucleic acid molecule, wherein the terminal nucleotides can further comprise one or more (e.g., 1, 2, 3, or 4) phosphorothioate internucleotide linkages, and wherein the nucleic acid optionally further comprises a terminal phosphate group, such as a 5′-terminal phosphate group.

In another embodiment, any modified nucleotides present in the single stranded nucleic acid molecules of the present invention comprise modified nucleotides having properties or -characteristics similar to naturally occurring ribonucleotides. For example, the invention features nucleic acid molecules including modified nucleotides having a Northern conformation (e.g., Northern pseudorotation cycle, see for example Saenger, Principles of Nucleic Acid Structure, Springer-Verlag ea., 1984). As such, chemically modified nucleotides present in the single stranded nucleic acid molecules of the present invention are preferably resistant to nuclease degradation while at the same time maintaining the capacity to mediate RNAi.

### Vectors, and Host Cells

The present invention also relates to vectors containing the polynucleotide of the present invention, and host cells comprising the same. The vector may be, for example, a phage, plasmid, viral, or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells. Methods of introducing RNAi reagents into cells using retroviral vectors are described in U.S. patent application Publication No. US20040033974, filed Aug. 19, 2002; which is hereby incorporated by reference herein in its entirety.

One embodiment of the invention provides an expression vector comprising a at least one nucleic acid sequence of the present invention in a manner that allows expression of the nucleic acid molecule.

Another embodiment of the present invention provides a mammalian cell comprising such an expression vector. The mammalian cell can be a human cell. The nucleic acid molecule of the expression vector can comprise a sense region and an antisense region. The antisense region can comprise sequence complementary to a RNA or DNA sequence encoding E2F1, NFkB, CREB-1, or CDC2A and the sense region can comprise sequence complementary to the antisense region. The nucleic acid molecule can comprise two distinct strands having complementary sense and antisense regions. The nucleic acid molecule can comprise a single strand having complementary sense and antisense regions.

In another aspect of the invention, nucleic acid molecules of the invention are expressed from transcription units inserted into DNA or RNA vectors. The recombinant vectors can be DNA plasmids or viral vectors. Nucleic acid expressing viral vectors can be constructed based on, but not limited to, adeno-associated virus, retrovirus, adenovirus, or alphavirus.

The recombinant vectors capable of expressing the nucleic acid molecules can be delivered as described herein, and persist in target cells. Alternatively, viral vectors can be used that provide for transient expression of nucleic acid molecules.

The present invention also encompasses vectors comprising the double stranded form, the sense strand, or the antisense strand of each of the RNAi reagents of the present invention. Specifically, the present invention encompasses the vectors disclosed in the following published patent applications:. U.S. Ser. No. 09/821,832, Filed Mar. 30, 2001; U.S. Ser. No. 10/255,568, filed Sep. 26, 2002; PCT International Application No. PCT/EP01/13968, Filed Nov. 29, 2001; and U.S. Publication No. US20030084471, filed Jan. 22, 2002. Additional methods and methods of use disclosed by these applications are hereby incorporated by reference herein in their entirety.

Yet another aspect of the present invention provides a method for attenuating expression of a target gene in cells, such cells may be cells in vitro, in vivo, or ex vivo, comprising introducing an expression vector having a “coding sequence” which, when transcribed, produces double stranded RNA (dsRNA) in the cell in an amount sufficient to attenuate expression of the target gene, wherein the dsRNA comprises a nucleotide sequence that hybridizes under stringent conditions to a nucleotide sequence of the target gene. In certain embodiments, the vector includes a single coding sequence for the dsRNA which is operably linked to (two) transcriptional regulatory sequences which cause transcription in both directions to form complementary transcripts of the coding sequence. In other embodiments, the vector includes two coding sequences which, respectively, give rise to the two complementary sequences which form the dsRNA when annealed. In still other embodiments, the vector includes a coding sequence which forms a hairpin. In certain embodiments, the vectors are episomal, e.g., and transfection is transient. In other embodiments, the vectors are chromosomally integrated, e.g., to produce a stably transfected cell line. Preferred vectors for forming such stable cell lines are described in U.S. Pat. No. 6,025,192 and PCT publication WO/9812339, which are incorporated by reference herein in their entirety.

Another aspect of the present invention provides a method for attenuating expression of a target gene in cells, such cells may be cells in vitro, in vivo, or ex vivo, comprising introducing an expression vector having a “non-coding sequence” which, when transcribed, produces double stranded RNA (dsRNA) in the cell in an amount sufficient to attenuate expression of the target gene. The non-coding sequence may include intronic or promoter sequence of the target gene of interest, and the dsRNA comprises a nucleotide sequence that hybridizes under stringent conditions to a nucleotide sequence of the promoter or intron of the target gene. In certain embodiments, the vector includes a single sequence for the dsRNA which is operably linked to (two) transcriptional regulatory sequences which cause transcription in both directions to form complementary transcripts of the sequence. In other embodiments, the vector includes two sequences which, respectively, give rise to the two complementary sequences which form the dsRNA when annealed. In still other embodiments, the vector includes a coding sequence which forms a hairpin. In certain embodiments, the vectors are episomal, e.g., and transfection is transient. In other embodiments, the vectors are chromosomally integrated, e.g., to produce a stably transfected cell line. Preferred vectors for forming such stable cell lines are described in U.S. Pat. No. 6,025,192 and PCT publication WO/9812339, which are incorporated by reference herein in their entirety.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., coding sequence), and/or to include genetic material (e.g., heterologous polynucleotide sequences) that is operably associated with the polynucleotides of the invention, and which activates, alters, and/or amplifies endogenous polynucleotides. For example, techniques known in the art may be used to operably associate heterologous control regions (e.g., promoter and/or enhancer) and endogenous polynucleotide sequences via homologous recombination, resulting in the formation of a new transcription unit (see, e.g., U.S. Pat. No. 5,641,670, issued Jun. 24, 1997; U.S. Pat. No. 5,733,761, issued Mar. 31, 1998; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

Oligonucleotides (e.g. certain modified oligonucleotides or portions of oligonucleotides lacking ribonucleotides) are synthesized using protocols known in the art, for example as described in Caruthers et al., 1992, Methods in Enzymology 211, 3 19, Thompson et al., International PCT Publication No. WO 99/54459, Wincott et al., 1995, Nucleic Acids Res. 23, 2677-2684, Wincott et al., 1997, Methods Mol. Bio., 74, 20 59, Brian et al., 1998, Biotechnol Bioeng, 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non limiting example, small scale syntheses are conducted on a 394 Applied Biosystems, Inc. synthesizer using a 0.2, umol scale protocol with a 2.5 min coupling step for 2′-O methylated nucleotides and a 45 sec. coupling step for 2′-deoxy nucleotides or 2′-deoxy 2′-fluoro nucleotides.

Alternatively, syntheses at the 0.2 Wool scale can be performed on a 96-well plate synthesizer, such as the instrument produced by Protogene (Palo Alto, Calif.) with minimal modification to the cycle. A 33-fold excess (60 AL of 0.11 M=6.6 drool) of 2′-O-methyl phosphoramidite and a 105-fold excess of S-ethyl tetrazole (60 AL of 0.25 M=15 drool) can be used in each coupling cycle of 2′-O-methyl residues relative to polymer-bound 5′-hydroxyl. A 22-fold excess (40 I1L of 0.11 M=4.4 drool) of deoxy phosphoramidite and a 70-fold excess of S-ethyl tetrazole (40 AL of 0.25 M=10 drool) can be used in each coupling cycle of deoxy residues relative to polymer-bound 5′-hydroxyl. Average coupling yields on the 394 Applied Biosystems, Inc. synthesizer, determined by calorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); and oxidation solution 10 is 16.9 mM I2, 49 mM pyridine, 9% water in THF (PERSEPTIVE_). Burdick & Jackson Synthesis Grade acetonitrile is used directly from the reagent bottle. S Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1, 1-dioxide, 15 0.05 M in acetonitrile) is used.

Deprotection of the DNA-based oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is 20 washed three times with 1.0 mL of EtOH:MeCN:H20/3:1:1, vortexed and the supernatant is then added to the first supernatant. The combined supernatants, containing the oligoribonucleotide, are dried to a white powder.

The method of synthesis used for RNA including certain nucleic acid molecules of the invention follows the procedure as described in Usman et al., 1987, J. Am. Chem. Soc., 25 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 umol scale protocol with a 7.5 min coupling step for alkylsilyl protected nucleotides and a 2.5 min coupling step for 2′-O-methylated nucleotides.

Alternatively, syntheses at the 0.2 Wool scale can be done on a 96-well plate synthesizer, such as the instrument produced by Protogene (Palo Alto, Calif.) with minimal modification to the cycle. A 33-fold excess (60 I1L of 0.11 M=6.6 drool) of 2′-O-methyl phosphoramidite and a 75-fold excess of S-ethyl tekazole (60 ILL of 0.25 M=15 5 drool) can be used in each coupling cycle of 2′-O-methyl residues relative to polymer-bound 5′-hydroxyl. A 66-fold excess (120 AL of 0.11 M=13.2 drool) of alkylsilyl (ribo) protected phosphoramidite and a 150-fold excess of S-ethyl tetrazole (120 I1L of 0.25 M=30 Stool) can be used in each coupling cycle of ribo residues relative to polymer-bound 5′-hydroxyl. Average coupling yields on the 394 Applied Biosystems, Inc. synthesizer, determined by calorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16. 9 nM I2, 49 mM pyridine, 9% water in THF (PERSEPTIVE_). Burdick & Jackson Synthesis Grade acetonitrile is used directly from the reagent bottle. S-Ethyltetrazole solution (0.25 M in acetonitrile) is made up from the solid obtained from American International Chemical, c. Alternately, for the introduction of phosphorothioate linkages, Beaucage reagent (3H-1,2-Benzodithiol-3-one 1,1-dioxideO.0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of 25 EtOH:MeCN:H20/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 AL of a solution of 1.5 mL N-methylpyrrolidinone, 750 pL TEA and 1 mL TEA.3HF to provide a 1.4 M HF concentration) and heated to 65° C.

After 1.5 h, the oligomer is quenched with 1.5 M NH4HCO3. Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 rnL) at 65° C. for 15 min. The vial is brought to rt. TEA.3HP (0.1 mL) is added and the vial is heated at 65° C. for 15 min. The sample is cooled at −20° C. and then quenched with 1.5 M NH4HCO3.

For purification of the trityl-on oligomers, the quenched NH4HCO3 solution is 5 loaded onto a C-18 containing cartridge that had been prewashed with acetonitrile followed by 50 mM TEAA. After washing the loaded cartridge with water, the RNA is detritylated with 0.5% TEA for 13 min. The cartridge is then washed again with water, salt exchanged with 1 M NaCl and washed with water again. The oligonucleotide is then eluted with 30% acetonitrile.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96-well format.

Alternatively, the nucleic acid molecules of the present invention can be synthesized separately and joined together post-synthetically, for example, by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Clean. 8, 204), or by hybridization following synthesis and/or deprotection.

The siNA molecules of the invention can also be synthesized via a tandem synthesis methodology, wherein both siNA strands are synthesized as a single contiguous oligonucleotide fragment or strand separated by a cleavable linker which is subsequently cleaved to provide separate siNA fragments or strands that hybridize and permit purification of the siNA duplex. The linker can be a polynucleotide linker or a non-nucleotide linker. The tandem synthesis of siNA as described herein can be readily adapted to both multiwell/multiplate synthesis platforms such as 96 well or similarly larger multi-well platforms.

The tandem synthesis of siNA as described herein can also be readily adapted to large scale synthesis platforms employing batch reactors, synthesis columns and the like.

A siRNA molecule can also be assembled from two distinct nucleic acid strands or fragments wherein one fragment includes the sense region and the second fragment includes the antisense region of the RNA molecule.

The nucleic acid molecules of the present invention can be modified extensively to 5 enhance stability by modification with nuclease resistant groups, for example, 2′-amino, 2′-C-allyl, 2′-fluoro, 2′-O-methyl, 2′-H (for a review see Usman and Cedergren, 1992, TIB5 17, 34; Usman et al., 1994, Nucleic Acids Symp. Ser. 31, 163). siRNA constructs can be purified by gel electrophoresis using general methods or can be purified by high pressure liquid chromatography (HPLC; see Wincott et al., supra, the totality of which is hereby incorporated herein by reference) and re-suspended in water.

Chemically synthesizing nucleic acid molecules with modifications (base, sugar 20 and/or phosphate) can prevent their degradation by serum ribonucleases, which can increase their potency (see e.g. Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,30O,074; and Burgin et al., supra; all of which are incorporated by reference herein). All of the above references describe various chemical modifications that can be made to the base, phosphate and/or sugar moieties of the nucleic acid molecules described herein.

Modifications that enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with 5 nuclease resistant groups, for example, 2′-amino, 2′-C-allyl, 2′-fluoro, 2′-O-methyl, 2′-O-allyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. 10 WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al.: International Publication PCT No. WO 93/15187; Sproat, U.S. Pat. No. 5,334,711 and Beigelman et al., 1995, J. Biol. Chew., 270, 25702; Beigelnan et al., International PCT publication No. WO 97/26270; Beigelman et al., U.S. Pat. No. 15 5,716,824; Usman et al., U.S. Pat. No. 5,627,053; Woolf et. al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Eanshaw and Gait, 1998, Biopolymers (Nucleic Acid Sciences), 48, 39-55; Verma and Eckstein, 1998, Ann. Rev. Biochem., 67, 99-134; and Burlina et al., 1997, Bioorg Med. Chem., 5, 1999-2010; 20 all of the references are hereby incorporated in their totality by reference herein). Such publications describe general methods and strategies to determine the location of incorporation of sugar, base and/or phosphate modifications and the like into nucleic acid molecules without modulating catalysis, and are incorporated by reference herein. In view of such teachings, similar modifications can be used as described herein to modify 25 the siRNA nucleic acid molecules of the instant invention so long as the ability of siRNA to promote RNAi is cells is not significantly inhibited.

While chemical modification of oligonucleotide internucleotide linkages with phosphorothioate, phosphorodithioate, and/or 5′-methylphosphonate linkages improves stability, excessive modifications can cause some toxicity or decreased activity.

Therefore, when designing nucleic acid molecules, the amount of these internucleotide linkages should be minimized. The reduction in the concentration of these linkages should lower toxicity, resulting in increased efficacy and higher specificity of these molecules.

Short interfering nucleic acid (siRNA) molecules having chemical modifications that maintain or enhance activity are provided. Such a nucleic acid is also generally more resistant to nucleases than an unmodified nucleic acid. Accordingly, the in vitro and/or in viva activity should not be significantly lowered. In cases in which modulation is the goal, therapeutic nucleic acid molecules delivered exogenously should optimally be stable within cells until translation of the target RNA has been modulated long enough to reduce the levels of the undesirable protein. This period of time varies between hours to days depending upon the disease state. Improvements in the chemical synthesis of RNA and DNA (Wincott et al., 1995, Nucleic Acids Res. 23, 2677; Caruthers et al., 1992, 10 Methods in Enzymology 211,3-19 (incorporated by reference herein)) have expanded the ability to modify nucleic acid molecules by introducing nucleotide modifications to enhance their nuclease stability, as described above.

In one embodiment, nucleic acid molecules of the invention include one or more (e.g. about 1, 2, 3, 4, 5, 6, 7, 8, 9, 10, or more) G-clamp nucleotides. A G-clamp 15 nucleotide is a modified cytosine analog wherein the modifications confer the ability to hydrogen bond both Watson-Crick and Hoogsteen faces of a complementary guanine within a duplex, see for example Lin and Matteucci, 1998, J: Am. Chem. Soc., 120, 8531-8532. A single G-clamp analog substitution within an oligonucleotide can result in substantially enhanced helical thermal stability and mismatch discrimination when hybridized to complementary oligonucleotides. The inclusion of such nucleotides in nucleic acid molecules of the invention results in both enhanced affinity and specificity to nucleic acid targets, complementary sequences, or template strands. In another embodiment, nucleic acid molecules of the invention include one or more (e.g. about 1, 2, 3, 4, 5, 6, 7, 8, 9, 10, or more) LNA “locked nucleic acid” nucleotides such as a 2′, 4′ 25 C methylene bicycle nucleotide (see for example Wengel et al., International PCT Publication No. WO 00/66604 and WO 99/14226).

In another embodiment, the invention features conjugates and/or complexes of siRNA molecules of the invention. Such conjugates and/or complexes can be used to facilitate delivery of siRNA molecules into a biological system, such as a cell. The conjugates and complexes provided by the instant invention can impart therapeutic activity by transferring therapeutic compounds across cellular membranes, altering the pharmacokinetics, and/or modulating the localization of nucleic acid molecules of the invention. The present invention encompasses the design and synthesis of novel conjugates and complexes for the delivery of molecules, including, but not limited to, small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers.

These compounds are expected to improve delivery and/or localization of nucleic acid molecules of the invention into a number of cell types originating from different tissues, in the presence or absence of serum (see Sullenger and Cech, U.S. Pat. No. 5,854,038).

Conjugates of the molecules described herein can be attached to biologically active molecules via linkers that are biodegradable, such as biodegradable nucleic acid linker molecules. The term “biodegradable linker” as used herein, refers to a nucleic acid or non nucleic acid linker molecule that is designed as a biodegradable linker to connect one molecule to another molecule, for example, a biologically active molecule to a siRNA molecule of the invention or the sense and antisense strands of a siRNA molecule of the invention. The biodegradable linker is designed such that its stability can be modulated for a particular purpose, such as delivery to a particular tissue or cell type. The stability of a nucleic acid-based biodegradable linker molecule can be modulated by using various chemistries, for example combinations of ribonucleotides, deoxyribonucleotides, and chemically-modified nucleotides, such as 2′-O-methyl, 2′-fluoro, 2′-amino, 2′-O-amino, 2′-C-allyl, 2′-O-allyl, and other 2′-modified or base modified nucleotides The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

Non-limiting examples of biologically active siRNA molecules either alone or in combination with other molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras,. siRNA, dsRNA, allozymes, aptamers, decoys and analogs thereof. Biologically active molecules of the invention also include molecules capable of modulating the pharmacokinetics and/or pharmacodynamics of other biologically active molecules, for example, lipids and polymers such as polyamides, polyamides, polyethylene glycol and other polyethers.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the sequence listing submitted herewith and the corresponding computer readable form are both incorporated herein by reference in their entireties.

The following examples are offered by way of illustration and not by way of limitation.

### EXAMPLES

### DESCRIPTION OF THE PREFERRED EMBODIMENTS

### Example 1

### Method of Selecing and Synthesizing the RNAi Regents of the present Invention

The sequence of the RNAi reagents of the present invention were chosen based upon the application of proprietary algorithms to the coding region of each of the target genes (e.g., E2F1, NFkB, CREB-1, and CDC2A) that incorporate both known rules and proprietary rules for designing such reagents. Identified RNAi reagents were cross-checked using BLAST searches against publicly available sequences databases to ensure each sequence was specific to the target sequence. At least four RNAi reagents were created for each target sequence. The efficacy of each RNAi reagent for inhibiting the expression of each target sequence was assessed as outlined elsewhere herein.

Each HPP Grade RNAi reagent was synthesized by QIAGEN (Valencia, Calif.) using proprietary TOM amidites at 20 nmol scale using. Each RNAi reagent of the present invention was synthesized in accordance with the methods outlined in the following U.S. patent: U.S. Pat. No. 5,986,084; which is hereby incorporated by reference herein in its entirety.

### Example 2

### Method of Transfecting Cultured Cells with the RNAi Reagents of the Present Invention

### RNAi transfection

RNAi transfection was done in 96 well plate. Briefly, Hela cell (ATCC) were seeded the day before the transfection at 26,000 cell/well in 125 ul of MEM *media plus 10% of FBS. Before transfection, a dilution of the Lipofectamine™ 2000 (INVITROGEN) was prepared. From the stock tube, a 1:25 dilution in Opti-MEM was made. The mixture was allowed to stand at room temperature for about 15 minutes. At the same time, a dilution of the siRNA duplexes from the 20 uM stock tube was prepared. The dilution was further diluted in Opti-MEM to make a final concentration of 240 nM. After the lipid was diluted for 15 minutes, equal volumes of the diluted lipid and the diluted siRNA duplexes were mixed together and incubated at room temperature for 20 minutes to allow the siRNA and the lipid to form complexes. Then, 25 μl of the mixed solution was added to the appropriate wells, pipetted up and down, and incubated at 37° C. for 48 hours.

### Example 3

### Method of Measuring the Effect of Transfecting Cultured Cells with the RNAi Reagents of the Present Invention on the Transcript Levels of each Target mRNA

### mRNA Isolation

mRNA was isolated according to the manufacturers instructions for the mRNA Catcher™ Protocol from SEQUITUR (Natick, Mass.).

### cDNA Synthesis

cDNA synthesis was performed by using a modified procedure outlined in the ABI TaqMan reverse transcription kit, No. N808-0234 from Applied Biosystems, Inc. (Foster City, Calif.). Briefly, the modified method was as follows: 19.25 ul of mRNA solution was used for cDNA synthesis. The reaction was performed in an ABI thermal cycler 9600 with one cycle as follows: 25° C., 10 min; 48° C., 40 min; and 95° C. for 5 min. The cDNA was keep at −20° C. until use.

### Quantitative RT-PCR

Oligonucleotide primers and TaqMan® probes for the E2F1, CREB, NFkB-p65, and CDC2A genes for the quantitative PCR experiments were purchased from Taqman® Assays-on-Demand™ Gene Expression Products (Applied Biosystems Inc.; Foster City, Calif.). The Taqman probe was labeled with Fluorescence dyes FAM and NFQ, respectively. To determine the relative expression levels of each gene in RNAi treated cell lines, 7.5 ul of each cDNA was subjected to Hot GoldDNA polymerase in 1× qPCR master mix (EUROGENTEC; Philadelphia, Pa.) in a final volume of 25 ul containing dNTPs, 5 mM MgCl2, Uracil-N-glycosylase, stabilizers, passive-reference, 0.9 M of each pair of primers, and 250 nM TaqMan® MGB probe. The thermal cycling conditions used were 50° C. for 2 min, 95° C. for 10 min, followed by 40 cycles at 95° C. for 15 s and at 60° C. for 1 min. All the reactions were performed at least in duplicate and analyzed using ABI Prism 7900HT detection system (Applied Biosystems; Foster City, Calif.). Data analysis of quantitative real-time RT-PCR values was as described according to the manufacturers instructions. The relative amount of mRNA in RNAi treated sample, normalized to internal control Human cyclophilin A (PPIA) or Human glyceraldehyde-3-phosphate dehydrogenase (GAPDH) and relative to a calibrator (GFP-B treated sample), was calculated by software SDS2.1 (Applied Biosystems).

The PPIA receptor-specific TaqMan primer set was obtained from Applied Biosystems (Foster City, Calif.) as an Assays-on-Demand(TM) Gene Expression Product (Assay ID No. Hs99999904_m1). The Assays-on-Demand(TM) Gene Expression Product for the PPIA receptor includes both forward and reverse primers specific to the PPIA transcript, in addition to a TaqMan probe that hybridizes to the resulting amplification product. The fluorescent reporter used for quantitation by the TaqMan probe was FAM. The context sequence to which the TaqMan probe was directed for the PPIA transcript was as follows:

CTGCACTGCCAAGACTGAGTGGTTG. (SEQ ID NO:39)

### Example 4

### Method of Assessing the Effect of Transfecting Cultured Cells with RNAi Reagents Directed Against the E2F1 Receptor on E2F1 Transcript Levels Using RT-PCR

The level of human E2F1 transcription factor 1 (E2F1; Genbank Accession No. NM_{—}005225) transcript in HeLa cells subsequent to transfection with E2F1-specific RNAi reagents was assessed using RT-PCR. HeLa cells were transfected with one of four E2F1-specific RNAi reagents according to the method outlined in Example 2 herein. The sequence of the plus and minus strand of each double-stranded E2F1-specific RNAi reagent is provided in Table 1 below. The intended target sequence within the E2F1 transcript is also provided for each RNAi reagent.

RT-PCR was performed as outlined in Example 3. The E2F1 receptor-specific TaqMan primer set was obtained from Applied Biosystems (Foster City, Calif.) as an Assays-on-Demand(TM) Gene Expression Product (Assay ID No. Hs00153451_m1). The Assays-on-Demand(TM) Gene Expression Product for the E2F1 receptor includes both forward and reverse primers specific to the E2F1 transcript, in addition to a TaqMan probe that hybridizes to the resulting amplification product. The fluorescent reporter used for quantitation by the TaqMan probe was FAM. The context sequence to which the TaqMan probe is directed was as follows:

TCCAGTGGCTGGGCAGCCACACCAC. (SEQ ID NO:36)

TABLE 1
Sequences of E2F1 RNAi reagents

| RNAi Reagent | | | |
|---|---|---|---|
| Name | E2F1 Target Sequence | Plus Strand Sequence | Minus Strand Sequence |
| BMS-E2F1-1* | AAACAAGGCCCGATC | r(ACAAGGCCCGAUCG | r(AACAUCGAUCGGG |
| | GATGTT (SEQ ID | AUGUU)d(TT) (SEQ ID | CCUUGU)d(TT) (SEQ |
| | NO:1) | NO:5) | ID NO:9) |
| BMS-E2F1-2* | AAGTCACGCTATGAG | r(GUCACGCUAUGAG | r(UGAGGUCUCAUAG |
| | ACCTCA (SEQ ID | ACCUCA)d(TT) (SEQ | CGUGAC)d(TT) (SEQ |
| | NO:2) | ID NO:6) | ID NO:10) |
| BMS-E2F1-3 | AAGCGGAGGCTGGA | r(GCGGAGGCUGGAC | r(UUCCAGGUCCAGCC |
| | CCTGGAA (SEQ ID | CUGGAA)d(TT) (SEQ | UCCGC)d(TT) (SEQ ID |
| | NO:3) | ID NO:7) | NO:11) |
| BMS-E2F1-4 | AAGCCGTGGACTCTT | r(GCCGUGGACUCUUC | r(UCUCCGAAGAGUCC |
| | CGGAGA (SEQ ID | GGAGA)d(TT) (SEQ ID | ACGGC)d(TT)(SEQ ID |
| | NO:4) | NO:8) | NO:12) |

*Asterisks indicate those RNAi reagents that were most efficient in knocking down the E2F1 transcript.

The results of the E2F1-specific RNAi reagent transfection on E2F1 transcript levels is provided in FIG. 1. As shown, the E2F1-specific RNAi reagents resulted in significant knockdown of E2F1 transcript levels. Reagents BMS-E2F1-1 and BMS-E2F1-2 were most effective in knocking down E2F1 transcript levels. These results indicate that the E2F1-specific RNAi reagents of the present invention are efficacious agents for inhibiting E2F1 expression and E2F1 function.

### Example 5

### Method of Assessing the Effect of Transfecting Cultured Cells with RNAi Reagents Directed Against the NFkB-p65 Polypeptide on NFkB-p65 Protein Levels Using Western Blots

The level of the Homo sapiens v-rel reticuloendotheliosis viral oncogene homolog A, nuclear factor of kappa light polypeptide gene enhancer in B-cells 3, p65 (avian) (RELA) (NFkB-p65; Genbank Accession No. NM_{—}006509) in HeLa cells subsequent to transfection with NFkB-p65-specific RNAi reagents was assessed at the protein level using western blotting. HeLa cells were transfected with one of four NFkB-p65-specific RNAi reagents according to the method outlined in Example 2 herein. The sequence of the plus and minus strand of each double-stranded NFkB-p65-specific RNAi reagent is provided in Table 2 below. The intended target sequence within the NFkB-p65 transcript is also provided for each RNAi reagent.

TABLE 2
Sequences of NFkB-p65 RNAi reagents

| RNAi Reagent | | |
|---|---|---|
| Name | Plus Strand Sequence | Minus Strand Sequence |
| BMS-NFkBp65-1 | GAUCAAUGGCUACACAGGATT | UCCUGUGUAGCCAUUGAUCTT |
| | (SEQ ID NO:13) | (SEQ ID NO:17) |
| BMS-NFkBp65-2* | CCUGGGAAUCCAGUGUGUGTT | CACACACUGGAUUCCCAGGTT |
| | (SEQ ID NO:14) | (SEQ ID NO:18) |
| BMS-NFkBp65-3* | GAGCAUCAUGAAGAAGAGUTT | ACUCUUCUUCAUGAUGCUCTT |
| | (SEQ ID NO:15) | (SEQ ID NO:19) |
| BMS-NFkBp65-4 | CAGCACAGACCCAGCUGUGTT | CACAGCUGGGUCUGUGCUGTT |
| | (SEQ ID NO:16) | (SEQ ID NO:20) |

*Asterisks indicate those RNAi reagents that were most efficient in knocking down the NFkB-p65 transcript.

### Transfection

One day prior to transfection, 2×10⁵ HeLa cells per well were seeded into a 24 well dish. The following day, cells were grown to 90-95% confluence. 4.5 uL of 20 uM stock RNAi was diluted in 50 uL Optimem in a polystyrene tube for each RNAi to be transfected (“tube A”) and gently mixed by tapping. In another polystyrene tube 2 uL Lipofectamine 2000 was combined with 50 uL Optimem (“tube B”) and mixed by gently tapping. Both mixtures were allowed to incubate at RT for 5′. 50 uL tube B was combined with the 50 uL for each tube A and mixed by gentle tapping. The mixtures were incubated at RT for 15′. 500 uL serum/antibiotic-free MEM was added to each tube to give a final RNAi concentration of 20-150 nM.

For cotransfections of RNAi with plasmid, 1 uL of 20 uM stock RNAi (final concentration of 33 nM) was used along with 1 ug vector DNA in tube A, and then the remainder of the transfection procedure was followed as outlined above.

The media from HeLa plates was removed and replaced with the 600 uL transfection mix and placed in a CO2 incubator at 37° C. with 5% C02 for 4-5 hours. The media was replaced with MEM containing 10% FBS after the incubation period.

Controls that were used in the transfection included a fluorescent control (1U/uL=20 uM) for assessing transfection efficiency (e.g., florescent BMS-FITC-RNAi), GFP B as a non-specific negative control (e.g., BMS-GFP-B), CDC2A as a normalizing knockdown control (e.g., BMS-CDC2A1-4), and an untransfected control receiving no DNA.

### Lysis

48 hours post-transfection, the media was aspirated and cells were washed 1× with approx. 500 uL cold 1× PBS per well. The wells were aspirated again and replaced with 100 uL cold RIPA containing protease inhibitors (1 mini BM protease inhibitor tablet/10 mL 1× RIPA). The plate was rocked and tapped a few times and placed at 4° C. for 10-15 minutes. The plate was tapped/rocked several more times.

Using a 200 uL pipetteman, the wells were aspirated 5-10 times and washed to ensure complete lysis and transfer of all cellular material. The lysate was transferred to an eppendorf tube and pipetted up and down 5-10 times. In cases where the sample was still viscous, pipetting was repeated several more times. Samples were spun down for 10′ at 14000 RPM 4° C., and either stored at −20° C. or prepared for loading.

### Western Blotting/Novex

The sample was prepared by combining 20 uL lysate with 3 uL reducing reagent and 7 uL 4× gel loading dye, heated at 70° C. for 10′, and then placed on ice. While samples were heating, the gel was prepared (e.g., 4-12% Bis-Tris gel) by removing comb and sealing tape. The gels were placed in a gel box and both the inner and outer chambers were filled with desired buffer (e.g., either 1× MES or MOPS-Add 50 mL 20× buffer to 950 mL dH20 for each gel box). (Note: Different gel percentages combined with either MES or MOPS allow for the desired separation of bands as outlined on the Invitrogen website or product manual).

600 uL of Oxidizing reagent was added to the inner chamber. Each well was washed out by blasting with 500 uL buffer. In well one, 5 uL Invitrogen's SeeBlue Plus2 (Invitrogen, Carlsbad, Calif.) marker was added. Samples were loaded in subsequent lanes, and the gel was run 200V for 45-50 minutes.

1× transfer buffer was prepared. The transfer buffer consisted of 50 mL 20× transfer buffer, Methanol (100 mL if transferring one gel, 200 mL if transferring 2 gels in the same apparatus) and dH20 to 1000 mL. The blotting pads were soaked in dH20 and then transfer buffer. The precut Hybond-ECL membrane (Amersham nitrocellulose) was presoaked in dH20 and then in transfer buffer. The end of a Biorad filter paper was cut off to match the size of the transfer membrane. In the instance where one gel was transferred, two blotting pads were placed into the blotting chamber. In the instance where two gels were transferred, only one blotting pad was placed in the blotting chamber. The filter paper was soaked in transfer buffer and carefully placed on blotting pad.

The gel cassette was opened using a cracking tool, and the top, bottom, and sides of the gel were cut off. The gel was briefly rinsed in transfer buffer and then placed on filter paper making sure that no air bubbles were present. The transfer membrane was placed on top again being careful to get rid of all bubbles. The filter paper was placed on top of the membrane and in addition to two blotting pads in the instance where one gel is being transferred, or one blotting pad in the instance where two gels are being transferred. Additional details may be obtained by referring to the Novex product manual. Gels are now ready for transfer.

The gel sandwich was squeezed together and placed in the transfer apparatus. The inner and outer chambers were filled with transfer buffer. The gel was transferred for 1 hour at 30V.

The membranes were removed and placed in Superblock (Pierce; Rockford, Ill.) and rocked at RT for a minimum of 1 hour to overnight. Primary antibody and normalizing antibody were diluted in a 1:10 mix of Superblock:1× PBS/0.3% Tween-20. The primary antibody for NFkB-p65 was obtained from BD transduction Laboratories (Catalog No. 610868). Membranes were incubated and rocked at RT in primary antibody for a minimum of 1 hour to overnight. Membranes were then washed thoroughly in 1× PBS/0.3% Tween-20, and rinsed 3× for 5′ in 1× PBS/0.3% Tween-20. During the final wash, HRP-conjugated secondary antibody was diluted in 1× PBS/0.3% Tween-20 and added to the membrane and rocked at RT for a minimum of 30′. The membranes were washed thoroughly 3× for 5′ in 1× PBS/0.3% Tween-20.

Membranes were removed from wash buffer and the excess buffer drained by holding the edge of the membranes on a paper towel. Membranes were placed on Saran Wrap that has been smoothed on the benchtop to remove air bubbles. Enough ECL reagent was added to cover the membrane for 1 minute. ECL was prepared by combining equal volumes of reagents 1 and 2. Membranes were removed and drained of excess ECL on a paper towel. Membranes were placed in-between two transparency sheets, being careful to smooth out air bubbles.

### Quantitation

Membranes were exposed using FluorS-Max. In addition to saving the PDQuest image, black, white, and contrast were adjusted. The image was cropped to get the desired area of each image and exported as a x.TIF image. The image was quantitated using Quantity One software (Bio-Rad; Hercules, Calif.). Using the volume tool, boxes were drawn around the bands of interest, in addition to background areas corresponding to an area adjacent to each band. Each band was normalized relative to the background area for each lane. Percent inhibition was determined by comparing the normalized band level of the control relative to each test band.

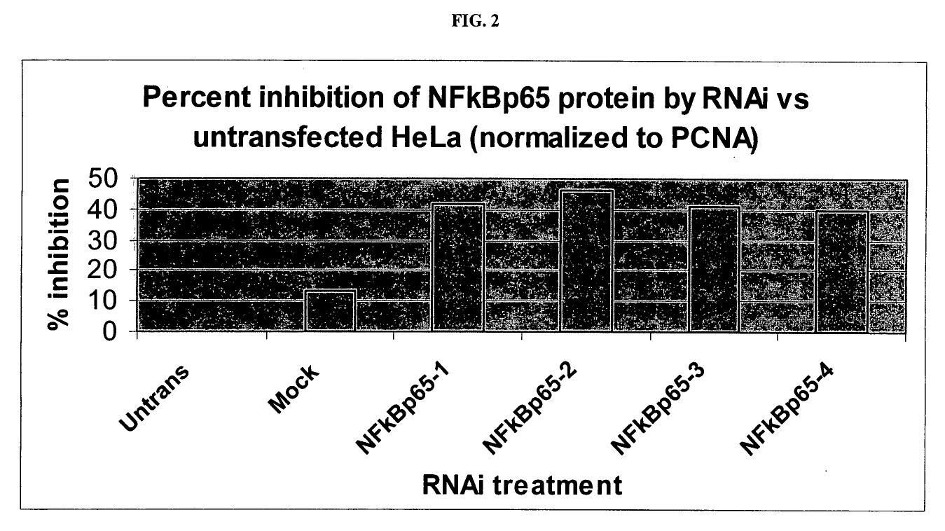

The results of the NFkB-p65-specific RNAi reagent transfection on NFkB-p65 transcript levels is provided in FIG. 2. As shown, the NFkB-p65-specific RNAi reagents resulted in significant knockdown of NFkB-p65 transcript levels in HeLa cells. Reagents BMS-NFkBp65-2 and BMS-NFkBp65-3 were most effective in knocking down NFkB-p65 transcript levels. These results indicate that the NFkB-p65-specific RNAi reagents of the present invention are efficacious agents for inhibiting NFkB-p65 expression and NFkB-p65 function.

### Example 6

### Method of Assessing the Effect of Transfecting Cultured Cells with RNAi Reagents Directed Against the CREB1 Polypeptide on CREB1 Transcript Levels Using RT-PCR

The level of human cAMP responsive element binding protein 1 (CREB1; Genbank Accession No. NM_{—}004379) transcript in HeLa cells subsequent to transfection with CREB1-specific RNAi reagents was assessed using RT-PCR. HeLa cells were transfected with one of four CREB1-specific RNAi reagents according to the method outlined in Example 2 herein. The sequence of the plus and minus strand of each double-stranded CREB1-specific RNAi reagent is provided in Table 3 below. The intended target sequence within the CREB1 transcript is also provided for each RNAi reagent.

RT-PCR was performed as outlined in Example 3. The CREB1-specific TaqMan primer set was obtained from Applied Biosystems (Foster City, Calif.) as an Assays-on-Demand(TM) Gene Expression Product (Assay ID No. Hs00231713_m1). The Assays-on-Demand(TM) Gene Expression Product for the CREB1 receptor includes both forward and reverse primers specific to the CREB1 transcript, in addition to a TaqMan probe that hybridizes to the resulting amplification product. The fluorescent reporter used for quantitation by the TaqMan probe was FAM. The context sequence to which the TaqMan probe is directed was as follows:

CCTTCCTACAGGAAAATTTTGAATG. (SEQ ID NO:37)

TABLE 3
Sequences of CREB1 RNAi reagents

| RNAi Reagent | | | |
|---|---|---|---|
| Name | CREB1 Target Sequence | Plus Strand Sequence | Minus Strand Sequence |
| BMS-CREB1-1 | AAGCCCAGCCACAGAU | R(GCCCAGCCACAGAU | R(UGGCAAUCUGUGGC |
| | UGCCA (SEQ ID | UGCCA)d(TT) (SEQ ID | UGGGC)d (TT) (SEQ ID |
| | NO:21) | NO:25) | NO:29) |
| BMS-CREB1-2* | AAUGGGCAGACAGUU | r(UGGGCAGACAGUUG | R(GACUUGAACUGUCU |
| | CAAGUC (SEQ ID | AAGUC)d(TT) (SEQ ID | GCCCA)d(TT) (SEQ ID |
| | NO:22) | NO:26) | NO:30) |
| BMS-CREB1-3 | AACUGAUUCCCAAAAG | r(CUGAUUCCCAAAAGC | R(CUUCGCUUUUGGGA |
| | CGAAG (SEQ ID | GAAG)d(TT) (SEQ ID | AUCAG)d(TT) (SEQ ID |
| | NO:23) | NO:27) | NO:31) |
| BMS-CREB1-4* | AACCAAGUUGUUGUU | r(CCAAGUUGUUGUUC | R(AGCUUGAACAACAA |
| | CAAGCU (SEQ ID | AAGCU)d(TT) (SEQ ID | CUUGG)d(TT) (SEQ ID |
| | NO:24) | NO:28) | NO:32) |

*Asterisks indicate those RNAi reagents that were most efficient in knocking down the CREB1 transcript.

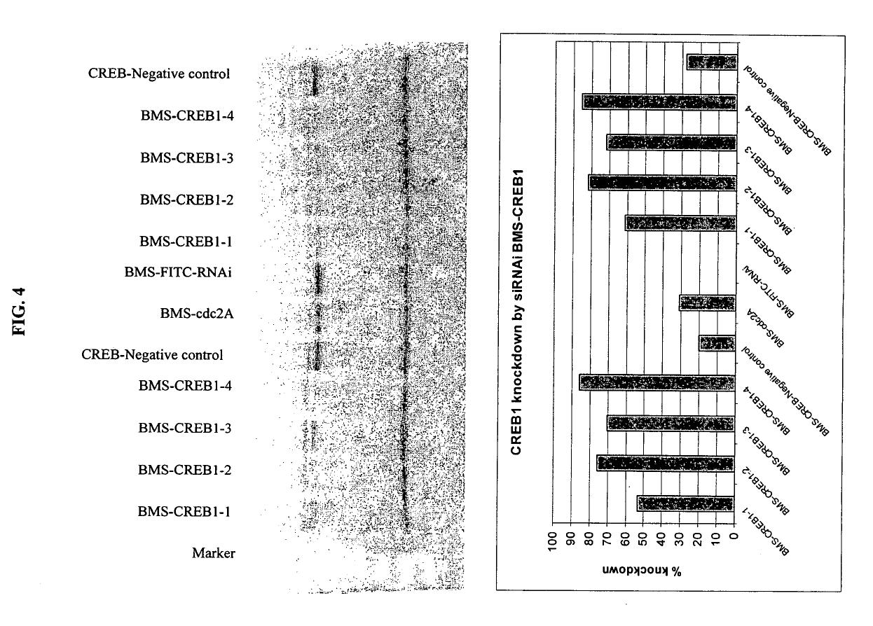

The results of the CREB1-specific RNAi reagent transfection on CREB1 transcript levels is provided in FIG. 4. As shown, the CREB1-specific RNAi reagents resulted in significant knockdown of CREB1 transcript levels in HeLa cells. Reagents BMS-CREB1-2 and BMS-CREB1-4 were most effective in knocking down CREB1 transcript levels.

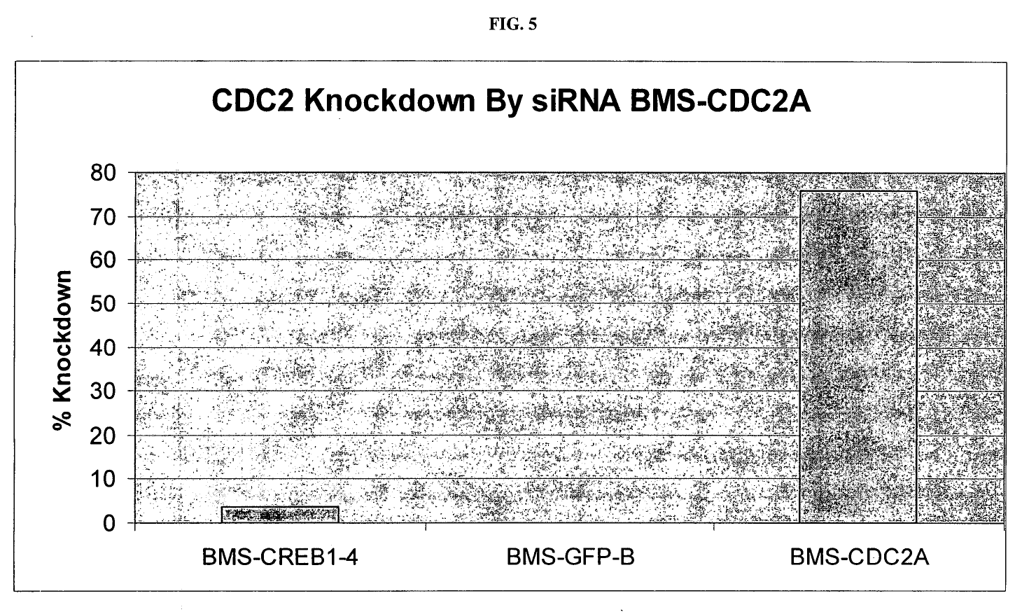

Importantly, the specificity of the RNAi reagents to CREB1 and CDC2A is noted. Inclusion of the CDC2-1-4 RNAi reagent with the CREB RT-PCR experiment illustrates that the CDC2-1-4 reagent had no effect on CREB transcript level. Moreover, the CDC2-1-4 RNAi reagent is capable of specifically knocking-down the level of CDC2A transcript levels as shown in FIG. 5 and discussed in Example 8. These results indicate that the CREB1-specific RNAi reagents of the present invention are efficacious agents for inhibiting CREB1 expression and CREB1 function.

### Example 7

### Method of Assessing the Effect of Transfecting Cultured Cells with RNAi Reagents Directed Against the CREB1 Polypeptide on CREB1 Protein Levels Using Western Blots

The level of the cAMP responsive element binding protein 1 (CREB1; Genbank Accession No. NM_{—}004379) in HeLa cells subsequent to transfection with CREB-specific RNAi reagents was also assessed at the protein level using western blotting. HeLa cells were transfected with one of four CREB-specific RNAi reagents according to the method outlined in Example 2 herein. The sequence of the plus and minus strand of each double-stranded CREB-specific RNAi reagent is provided in Table 2 as shown in Example 6.

### Transfection

One day prior to transfection, 2×10⁵ HeLa cells per well were seeded into a 24 well dish. The following day, cells were grown to 90-95% confluence. 4.5 uL of 20 uM stock RNAi was diluted in 50 uL Optimem in a polystyrene tube for each RNAi to be transfected (“tube A”) and gently mixed by tapping. In another polystyrene tube 2 uL Lipofectamine 2000 was combined with 50 uL Optimem (“tube B”) and mixed by gently tapping. Both mixtures were allowed to incubate at RT for 5′. 50 uL tube B was combined with the 50 uL for each tube A and mixed by gentle tapping. The mixtures were incubated at RT for 15′. 500 uL serum/antibiotic-free MEM was added to each tube to give a final RNAi concentration of 20-150 nM.

For cotransfections of RNAi with plasmid, 1 uL of 20 uM stock RNAi (final concentration of 33 nM) was used along with lug vector DNA in tube A, and then the remainder of the transfection procedure was followed as outlined above.

The media from HeLa plates was removed and replaced with the 600 uL transfection mix and placed in a CO2 incubator at 37° C. with 5% C02 for 4-5 hours. The media was replaced with MEM containing 10% FBS after the incubation period.

Controls that were used in the transfection included a fluorescent control (1U/uL=20 uM) for assessing transfection efficiency (e.g., florescent BMS-FITC-RNAi), GFP B as a non-specific negative control (e.g., BMS-GFP-B), CDC2A as a normalizing knockdown control (e.g., BMS-CDC2A1-4), and an untransfected control receiving no DNA.

### Lysis

48 hours post-transfection, the media was aspirated and cells were washed 1× with approx. 500 uL cold 1× PBS per well. The wells were aspirated again and replaced with 100 uL cold RIPA containing protease inhibitors (1 mini BM protease inhibitor tablet/10 mL 1× RIPA). The plate was rocked and tapped a few times and placed at 4° C. for 10-15 minutes. The plate was tapped/rocked several more times.

Using a 200 uL pipetteman, the wells were aspirated 5-10 times and washed to ensure complete lysis and transfer of all cellular material. The lysate was transferred to an eppendorf tube and pipetted up and down 5-10 times. In cases where the sample was still viscous, pipetting was repeated several more times. Samples were spun down for 10′ at 14000 RPM 4° C., and either stored at −20° C. or prepared for loading.

### Western Blotting/Novex

The sample was prepared by combining 20 uL lysate with 3 uL reducing reagent and 7 uL 4× gel loading dye, heated at 70° C. for 10′, and then placed on ice. While samples were heating, the gel was prepared (e.g., 4-12% Bis-Tris gel) by removing comb and sealing tape. The gels were placed in a gel box and both the inner and outer chambers; were filled with desired buffer (e.g., either 1× MES or MOPS-Add 50 mL 20× buffer to 950 mL dH20 for each gel box). (Note: Different gel percentages combined with either MES or MOPS allow for the desired separation of bands as outlined on the Invitrogen website or product manual).

600 uL of Oxidizing reagent was added to the inner chamber. Each well was washed out by blasting with 500 uL buffer. In well one, 5 uL Invitrogen's SeeBlue Plus2 (Invitrogen, Carlsbad, Calif.) marker was added. Samples were loaded in subsequent lanes, and the gel was run 200V for 45-50 minutes.

1× transfer buffer was prepared. The transfer buffer consisted of 50 mL 20× transfer buffer, Methanol (100 mL if transferring one gel, 200 mL if transferring 2 gels in the same apparatus) and dH20 to 1000 mL. The blotting pads were soaked in dH20 and then transfer buffer. The precut Hybond-ECL membrane (Amersham nitrocellulose) was presoaked in dH20 and then in transfer buffer. The end of a Biorad filter paper was cut off to match the size of the transfer membrane. In the instance where one gel was transferred, two blotting pads were placed into the blotting chamber. In the instance where two gels were transferred, only one blotting pad was placed in the blotting chamber. The filter paper was soaked in transfer buffer and carefully placed on blotting pad.

The gel cassette was opened using a cracking tool, and the top, bottom, and sides of the gel were cut off. The gel was briefly rinsed in transfer buffer and then placed on filter paper making sure that no air bubbles were present. The transfer membrane was placed on top again being careful to get rid of all bubbles. The filter paper was placed on top of the membrane and in addition to two blotting pads in the instance where one gel is being transferred, or one blotting pad in the instance where two gels are being transferred. Additional details may be obtained by referring to the Novex product manual. Gels are now ready for transfer.

The gel sandwich was squeezed together and placed in the transfer apparatus. The inner and outer chambers were filled with transfer buffer. The gel was transferred for 1 hour at 30V.

The membranes were removed and placed in Superblock (Pierce; Rockford, Ill.) and rocked at RT for a minimum of 1 hour to overnight. Primary antibody and normalizing antibody were diluted in a 1:10 mix of Superblock:1× PBS/0.3% Tween-20. Membranes were incubated and rocked at RT in primary antibody for a minimum of 1 hour to overnight. Membranes were then washed thoroughly in 1× PBS/0.3% Tween-20, and rinsed 3× for 5′ in 1× PBS/0.3% Tween-20. During the final wash, HRP-conjugated secondary antibody was diluted in 1× PBS/0.3% Tween-20 and added to the membrane and rocked at RT for a minimum of 30′. The membranes were washed thoroughly 3× for 5′ in 1× PBS/0.3% Tween-20.

Membranes were removed from wash buffer and the excess buffer drained by holding the edge of the membranes on a paper towel. Membranes were placed on Saran Wrap that has been smoothed on the benchtop to remove air bubbles. Enough ECL reagent was added to cover the membrane for 1 minute. ECL was prepared by combining equal volumes of reagents 1 and 2. Membranes were removed and drained of excess ECL on a paper towel. Membranes were placed in-between two transparency sheets, being careful to smooth out air bubbles.

### Quantitation

Membranes were exposed using FluorS-Max. In addition to saving the PDQuest image, black, white, and contrast were adjusted. The image was cropped to get the desired area of each image and exported as a x.TIF image. The image was quantitated using Quantity One software (Bio-Rad; Hercules, Calif.). Using the volume tool, boxes were drawn around the bands of interest, in addition to background areas corresponding to an area adjacent to each band. Each band was normalized relative to the background area for each lane. Percent inhibition was determined by comparing the normalized band level of the control relative to each test band.

The results of the CREB1-specific RNAi reagent transfection on CREB1 protein levels is provided in FIG. 4. As shown, the CREB1-specific RNAi reagents resulted in significant knockdown of CREB1 protein levels in HeLa cells, with the BMS-CREB1-2 and BMS-CREB1-4 reagents being most effective in knocking down CREB1 protein levels. These results further indicate that the CREB1-specific RNAi reagent of the present invention is an efficacious agent for inhibiting CREB1 expression and CREB1 function.

### Example 8

### Method of Assessing the Effect of Transfecting Cultured Cells with RNAi Reagents Directed Against the CDC2A Polypeptide on CDC2A Transcript Levels Using RT-PCR

The level of human cell division cycle 2, G1 to S and G2 to M protein (CDC2A; Genbank Accession No. NM_{—}001786) transcript in HeLa cells subsequent to transfection with CDC2A-specific RNAi reagents was assessed using RT-PCR. HeLa cells were transfected with a CDC2A-specific RNAi reagent according to the method outlined in Example 2 herein. The sequence of the plus and minus strand of the double-stranded CDC2A-specific RNAi reagent is provided in Table 4 below. The intended target sequence within the CDC2A transcript is also provided for each RNAi reagent.

RT-PCR was performed as outlined in Example 3. The CDC2A-specific TaqMan primer set was obtained from Applied Biosystems (Foster City, Calif.) as an Assays-on-Demand(TM) Gene Expression Product (Assay ID No. Hs00176469_m1). The Assays-on-Demand(TM) Gene Expression Product for the CDC2A receptor includes both forward and reverse primers specific to the CDC2A transcript, in addition to a TaqMan probe that hybridizes to the resulting amplification product. The fluorescent reporter used for quantitation by the TaqMan probe was FAM. The context sequence to which the TaqMan probe is directed was as follows:

AATAAGCCGGGATCTACCATACCCA. (SEQ ID NO:38)

TABLE 4
Sequences of CDC2A RNAi reagents.

| RNAi | | | |
|---|---|---|---|
| Reagent | | | |
| Name | CDC2A Target Sequence | Plus Strand Sequence | Minus Strand Sequence |
| BMS- | AAG GGG TTC CTA | r(GGG GUU CCU AGU | r(UUG CAG UAC UAG |
| CDC2A1-4* | GTA CTG CAA (SEQ ID | ACU GCA A)d(TT) (SEQ | GAA CCC C)d(TT) (SEQ |
| | NO:33) | ID NO:34) | ID NO:35) |

*Asterisks indicate those RNAi reagents that were most efficient in knocking down the CDC2A transcript.

The results of the CDC2A-specific RNAi reagent transfection on CDC2A transcript levels is provided in FIG. 5. As shown, the CDC2A-specific RNAi reagents resulted in significant knockdown of CDC2A transcript levels in HeLa cells. The BMS-CDC2A1-4 reagent was most effective in knocking down CDC2A transcript levels. These results indicate that the CDC2A-specific RNAi reagent of the present invention is an efficacious agent for inhibiting CDC2A expression and CDC2A function.

### Example 9

### Method of Assessing the Effect of Transfecting Cultured Cells with RNAi Reagents Directed Against the E2F1 Polypeptide on Nuclear Fragmentation Using DAPI-Staining on a Cellomics Platform

Although the ability of the E2F1-directed RNA reagents E2F1-1, E2F1-2, E2F1-3, and E2F1-4, to downregulate the level of E2F1 expressed in cells has been demonstrated (see FIG. 1), the inventors sought to assess whether downregulation of E2F1 by these reagents resulted in the same cellular manifestations (e.g., cell cycle arrest at the G2/M checkpoint, apoptosis, etc.) as has been observed by other E2F1 inhibiting reagents.

Thus, assays were designed to detect the percentage of cells undergoing nuclear fragmentation and/or nuclear swelling subsequent to transfecting A549 cells with each of the E2F1-directed RNAi reagents.

The assay was performed as follows:

### siRNA Transfections with E2F1-directed RNAi Reagents

1500 A549 cells per well were plated in a volume of 80 ul with RPMI 1640 medium containing 3.3% FBS on 96-well-plates one day prior to transfection. Transfections were set up in quadruplicates with siRNA at 25 nM, Lipofectamine 2000 (“LF2K”) at 0.8 ug/ml and FBS at 2.6%. siRNA to Luciferase-4 (Luc-4) served as a negative control, while wells containing Lipofectamine 2000 and wells receiving no treatment at all were included to monitor for transfection toxicity. Briefly, stock solutions of 0.25 uM siRNA (10×) were prepared using OPTI-MEM I followed by incubation for 5-10 minutes at room temperature. Meanwhile, stock solution of Lipofectamine 2000, 8 ug/ml LF2K (10×), was prepared with OPTI-MEM I followed by incubation for 5-10 minutes at room temperature. Equal volumes of 10× siRNA and 10× LF2K were mixed together, and incubated for 25 minutes at room temperature. 20 ul of the mixture was added to each well containing the A549 cells. Cells were incubated with each siRNA reagent for 72 hours prior to analysis.

### Fixation and Immuno-Cytochemistry

Live cells were stained for 10 minutes with TOTO-3 iodide at a final concentration of 0.25 uM at 37 degree subsequent to transfection. Cells were then fixed with pre-warmed formaldehyde (final 2%) for 15 minutes at room temperature. Cells were washed three times with 200 ul DPBS per well to remove the formaldehyde. Cells were blocked overnight at 4 degrees in blocking buffer. Cells were incubated with primary antibodies for 45 minutes at room temperature (dilutions were as follows: 1:2000 with anti-alpha-tubulin and 1:300 anti-active caspase-3). Cells were washed 3 times with 200 ul DPBS per well to remove excess antibody. Cells were then incubated with secondary antibodies (dilutions were as follows: 1:1200 for both Alexa-488 goat anti-rabbit IgG and Alexa-555 goat anti-mouse IgG), and DAPI (4 ug/ml) for 45 minutes at room temperature in the blocking buffer. Cells were washed 3 times with 200 ul DPBS per well to remove the excess antibodies and dye. The final 200 ul DPBS was maintained in the wells and then each plate was sealed for imaging.

### Reagents Utilized

Blocking buffer: 1× DPBS+1% BSA+0.25% Triton X-100

1× DPBS: Cat. No 21-031-CV, Cellgro

BSA: Cat. No. A-7906, Sigma

Triton X-100: cat. No. T8787, Sigma

10% Formaldehyde, Ultra-pure EM grade: Cat. No. 04018 , Polgysciences, Inc.

Anti-alpha-tubulin (clone DM IA): Cat. No. T-9026, Sigma

Anti-ACTIVE Caspase-3 pAb: Cat. No. G748 1, Promega

Alexa Fluor 488, goat anti-rabbit IgG(H+L): Cat. No. A11034, Invitrogen

Alexa Fluor 555 F(ab′)2 fragment of goat anti-mouse IgG(H+L): Cat. No. A-21425, Invitrogen

TOTO-3 iodide 642/660 nM: cat. No. T3604, Invitrogen

DAPI: Cat. No. D-21490, Invitrogen

OPTI-MEM I: Cat. No. 31985-088, Invitrogen

Lipofectamine 2000: Cat. No. 1232557, Invitrogen

RPMI-1640: cat. No. 11875-085, Invitrogen

### Image Acquisition

Images of fixed/stained cells from 4 different fluorescent channels simultaneously were obtained for each well using ArrayScan4.0 software (Cellomics, Inc., Pittsburgh) on an ArrayScan HCS Reader (Cellomics, Inc., Pittsburgh). Around 1800-2200 cells were collected from each well using 10× objective lens and “Target Activation Application” using the following channel parameters:
Ch1 (nucleus): 0.117 second exposure with XF93-Hoechst filter
Ch1 (a-tubulin): 0.2 second exposure with XF93-TRITC filter
Ch3 (active caspase-3): 0.6 second exposure with XF100-GFP filter
Ch4 (TOTO-3): 1 second exposure with XF110-Cye5 sensitive filter

### Gating Criteria for Analysis of Acquired Images

Live cells: caspase-3 signal<69.7, TOTO3 signal<350;

Live cells with nucleus Area: 130-350, P2A=1-2.5; LWR=1=2;

Fragmented and/or swelled nucleus: Nucleus Area: 130-350, P2A:1-2.5; LWR: 1-22, DAPI total intensity<51000.

The percent of total cells exhibiting fragmentation and swelling of the nucleus cells were defined as the population of cells that were capase-3 negative and TOTO-3 negative yet displayed an enlarged nucleus. These cells did not display an increase in DNA content. The mean signals of all parameters (e.g., % fragmentation, caspase-3 level, alpha-tubulin level, TOTO-3 level, DAPI level) from each well were normalized to the mean of 15 no treatment wells. DAPI is a stain that binds to the minor groove of DNA and can be used to directly measure the level of DNA in a sample since DAPI intensity is correlative with the amount of intact DNA. TOTO-3 iodide is a cell impermeable dye that is useful in assessing the integrity of the cell membrane since aberrations of the latter permit TOTO-3 into the cell resulting in significant increases in TOTO-3 measured relative to cells with intact cell membranes. TOTO-3 is useful for detecting apoptotic cells since such cells undergo significant cell membrane and nuclear fragmentation, with the former contributing to high levels of TOTO-3 staining. Caspase-3 is a key protein involved in the initiation of events leading up to the induction of apoptosis. The higher the level of caspase-3 in a cell, the further along the cell is in the apoptotic pathway.

The results of this experiment are provided in FIG. 6. As shown, transfection of cells with each of the E2F1-directed RNAi reagents resulted in a significant increase in the number of cells exhibiting nuclear fragmentation and/or swelling relative to the negative controls. The results clearly indicate that E2F1 has been downregulated as a consequence of transfection with the E2F1-directed RNAi reagents of the present invention on account of the increased percent of cells exhibiting nuclear fragmentation and/or nuclear swelling—both of the latter being classic markers of apoptosis. Note that the results for the E2F1 RNAi reagent in FIG. 6 were based upon the measurement from a single well, while all other results were performed in quadruplicate.

Dark field illumination and fluorescent images of cells transfected with E2F1-directed RNA reagent (“E2F1-3”) and negative control RNAi reagent (“Luc-4”) are provided in FIG. 8. The images clearly show several cells with 2 nuclei, nuclear swelling, and/or nuclear fragmentation in the E2F1-directed RNA reagent (“E2F1-3”), while no cells with these aberrations were detected in the negative control cells. Additionally, the nuclei of cells transfected with E2F1-directed RNA reagent (“E2F1-3”) also exhibited weak DAPI intensity compared to nuclei of the Luc-4 RNAi transfected cells. Results from the other E2F1-directed RNA reagents were similar to that observed for E2F1-3. Images of the latter results are not shown, but the results are summarized in FIG. 9.

FIG. 9 also provides a table with a quantitative summery of the results of each of these experiments. The table contains the Mean fold change or % of control and their standard deviation (“SD”) in each parameter, using “no treatment” wells as base line. In addition to the experiments described above and elsewhere herein, A549 cells were also transfected with XIAP (X-linked Inhibitor of Apoptosis Protein) according to the same conditions described above. XIAP-directed RNAi reagent was used as a positive control, since cells that have lost XIAP are known to undergo apoptosis (LaCasse and Reed: IAP family proteins-suppressors of apoptosis. Genes Dev 1999:13239-52). In general, all of the cells transfected with the E2F1-directed RNAi reagents exhibited significant increases in the level of caspase-3, alpha-tubulin, and TOTO-3 detected—all of which are consistent with the induction of apoptosis. Cells transfected with the E2F1-3 RNAi reagent exhibited the greatest level of apoptotic induction. Cell counts for E2F1-3 RNAi reagent transfected cells were less than ⅓ of the “no treatment” well and displayed a huge increase in the capase-3, TOTO-3 and α-tubulin signals. Cells transfected with E2F1-1, E2F1-2, and E2F1-4 RNAi reagents displayed more moderate apoptosis phenotypes.

Combined with the increased number of cells exhibiting nuclear fragmentation and swelling, in addition to the downregulation of cyclinD1 and CDC2 (see FIGS. 10 and 11), it is clear that the cells transfected with the E2F1-directed RNAi reagent induce apoptosis through the disruption of the cell cycle.

### Example 10

### Method of Assessing the Effect of Transfecting Cultured Cells with RNAi Reagents Directed Against the E2F1 Polypeptide on Nuclear Fragmentation Using DAPI-Staining on a Cellomics Platform

In an effort to provide additional supporting evidence that the E2F1-directed RNA reagents E2F1-1, E2F1-2, E2F1-3, and E2F1-4, were capable of inhibiting cell cycle arrest at the G2/M checkpoint, as a consequence of downregulating E2F1, the inventors sought to quantitate the DNA density of cells transfected with the E2F1-directed RNA reagents.

The assay utilized the same siRNA transfection, fixation, immuno-cytochemistry, imaging, and gating protocols described in Example 9, and involved measuring the total intensity of DAPI staining in the nucleus, which is directly correlative to the level of DNA in the nucleus on account of DAPI binding specifically to the minor groove of DNA. Experiments were performed in duplicate, and the quantitative level of DAPI staining measured in each nucleus was used to create a histogram. E2F1-3 RNAi reagent was tested, along with the negative control RNAi reagent, Luc-4.

The results of this experiment are provided in FIG. 7. As shown, transfection of cells with E2F1-directed RNAi reagent resulted in a significant increase in the number of cells exhibiting nuclear fragmentation and/or swelling relative to the negative control. The results clearly indicates cell cycle disruption in A549-cells treated with E2F1-directed RNAi reagent as a consequence of the loss of E2F1 function. Additionally, the results indicate that cells transfected with the E2F1-directed RNAi reagent caused a large increase in the G2/M populations compared to the Luc-4 controls. Specifically, the majority of the G2/M population of cells contained 2 nuclei which is an indication of a cytokinesis defect. This result is similar to that observed with other methods of disrupting E2F1 such as observed using an oligo decoy as described in U.S. Ser. No. US20020052333, filed on Apr. 19, 2001; and U.S. Ser. No. US20020128217.

Results from the other E2F1-directed RNA reagents of the present invention were similar to that observed for E2F1-3 (data not shown).

### Example 11

### Method of Assessing the Effect of Transfecting Cultured Cells with RNAi Reagents Directed Against the E2F1 Receptor on Cyclin D1 Transcript Levels Using RT-PCR

The level of human Cyclin D1 (Cyclin D1; Genbank Accession No. NM_{—}053056) transcript in HeLa cells subsequent to transfection with E2F1-specific RNAi reagents was assessed using RT-PCR. HeLa cells were transfected with one of four E2F1-specific RNAi reagents according to the method outlined in Example 1 herein. The sequence of the plus and minus strand of each double-stranded E2F1-specific RNAi reagent is provided in Table 1, supra. The intended target sequence within the E2F1 transcript is also provided for each RNAi reagent.

RT-PCR was performed as outlined in Example 2. The Cyclin D1-specific TaqMan primer set was obtained from Applied Biosystems (Foster City, Calif.) as an Assays-on-Demand(TM) Gene Expression Product (Assay ID No. Hs00277039_m1). The Assays-on-Demand(TM) Gene Expression Product for the Cyclin D1 includes both forward and reverse primers specific to the cyclin D1 transcript, in addition to a TaqMan probe that hybridizes to the resulting amplification product. The fluorescent reporter used for quantitation by the TaqMan probe was FAM. The context sequence to which the TaqMan probe was directed is as follows:

CCGAGGAGCTGCTGCAAATGGAGCT. (SEQ ID NO:40)

The results of the E2F1-specific RNAi reagent transfection on cell cycle regulatory gene CyclinD1 expression levels is provided in FIG. 10. As shown, the E2F1-specific RNAi reagents inhibit cell cycle regulatory gene CyclinD1 expression levels significantly. Reagents BMS-E2F1-1 and BMS-E2F1-3 were most effective in inhibition of Cyclin D1 transcript levels. These results indicate that the E2F1-specific RNAi reagents of the present invention are efficacious agents for inhibiting E2F1 expression and selectively inhibited the expression of targeted cell cycle regulatory gene CyclinD1 transactivated by E2F1.

### Example 12

### Method of Assessing the Effect of Transfecting Cultured Cells with RNAi Reagents Directed Against the E2F1 Receptor on CDC2 Transcript Levels Using RT-PCR

The level of human cell division cycle 2, G1 to S and G2 to M protein (CDC2A; Genbank Accession No. NM_{—}001786) transcript in HeLa cells subsequent to transfection with E2F1-specific RNAi reagents was assessed using RT-PCR. HeLa cells were transfected with one of four E2F1-specific RNAi reagents according to the method outlined in Example 1 herein. The sequence of the plus and minus strand of each double-stranded E2F1-specific RNAi reagent is provided in Table 1, supra. The intended target sequence within the E2F1 transcript is also provided for each RNAi reagent.

RT-PCR was performed as outlined in Example 2. The CDC2A-specific TaqMan primer set was obtained from Applied Biosystems (Foster City, Calif.) as an Assays-on-Demand(TM) Gene Expression Product (Assay ID No. Hs00176469_m1). The Assays-on-Demand(TM) Gene Expression Product for the CDC2A receptor includes both forward and reverse primers specific to the CDC2A transcript, in addition to a TaqMan probe that hybridizes to the resulting amplification product. The fluorescent reporter used for quantitation by the TaqMan probe was FAM. The context sequence to which the TaqMan probe is directed was as follows:

CTGCACTGCCAAGACTGAGTGGTTG. (SEQ ID NO:38)

The results of the E2F1-specific RNAi reagent transfection on cell cycle regulatory gene cdc2 expression levels is provided in FIG. 11. As shown, the E2F1-specific RNAi reagents inhibit cell cycle regulatory gene cdc2 expression levels significantly. Reagent BMS-E2F1-3 was most effective in inhibition of cdc2 transcript levels. As a positive control, Cdc2 specific RNAi (SEQ ID NO: 33, SEQ ID NO:34, SEQ ID NO:35) was included to show similar downregulation of cdc2 expression by RNAi reagent specific to cdc2 These results indicate that the E2F1-specific RNAi reagents of the present invention are efficacious agents for inhibiting E2F1 expression and selectively inhibited the expression of targeted cell cycle regulatory gene cdc2 transactivated by E2F1.

1. Method of inhibiting the expression of an E2F1 polypeptide in a cell or tissue comprising the step of contacting said cell or tissue with an RNAi reagent member selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12; under conditions in which the expression level of said E2F1 polypeptide is inhibited.

2. Method of inhibiting the expression of a NFkB polypeptide in a cell or tissue comprising the step of contacting said cell or tissue with an RNAi reagent member selected from the group consisting of: SEQ ID NO:13, SEQ ID NO:14, SEQ ID NO:15, SEQ ID NO:16, SEQ ID NO:17, SEQ ID NO:18, SEQ ID NO:19, and SEQ ID NO:20; under conditions in which the expression level of said NFkB polypeptide is inhibited.

3. Method of inhibiting the expression of a CREB-1 polypeptide in a cell or tissue comprising the step of contacting said cell or tissue with an RNAi reagent member selected from the group consisting of: SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, and SEQ ID NO:32; under conditions in which the expression level of said CREB-1 polypeptide is inhibited.

4. Method of inhibiting the expression of a CDC2A polypeptide in a cell or tissue comprising the step of contacting said cell or tissue with an RNAi reagent member selected from the group consisting of: SEQ ID NO:33, SEQ ID NO:34, and SEQ ID NO:35; under conditions in which the expression level of said CDC2A polypeptide is inhibited.

5. The method according to a member of the group consisting of: claim 1, claim 2, claim 3, and claim 4; wherein said RNAi reagent is a member of group consisting of: single stranded; and double stranded.

6. The method according to a member of the group consisting of: claim 1, claim 2, claim 3, and claim 4; wherein said RNAi reagent is comprised of a member of the group consisting of: deoxyribonucleotides; ribonucleotides; both deoxyribonucleotides and ribonucleotides; DNA; RNA; and RNA/DNA chimera.

7. The method according to a member of the group consisting of: claim 1, claim 2, claim 3, and claim 4; wherein said contacting step is performed according to a condition selected from a member of the group consisting of: in vitro, in vivo, and ex vivo.

8. The method according to a member of the group consisting of: claim 1, claim 2, claim 3, and claim 4; wherein said cell or tissue is selected from a member of the group consisting of: mammalian, and human.

9. A method of treating, ameliorating, or preventing a disorder comprising the step of administering to a cell, tissue, and/or subject, a pharmaceutically effective amount of an RNAi reagent selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO: 14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, and SEQ ID NO:35; wherein said cell, tissue, and/or subject is mammalian and/or human.

10. The method according to claim 9 wherein said disorder is selected from the group consisting of: inflammatory disorders, intimal hyperplasia, angiogenesis, neoplasia, immune disorders, neurological disorders, viral infections, disorders associated with E2F, disorders associated with aberrant E2F1 activity and/or expression, cell cycle disorders, cell cycle disorders associated with aberrant function of the S-phase check point, cell cycle disorders associated with aberrant function of the G2/S-phase check point, disorders associated with p53-dependent apoptosis, disorders associated with p53-independent apoptosis, cell cycle disorders associated with aberrant cyclin D1 regulation and/or function, cell cycle disorders associated with aberrant CDC2A regulation and/or function, cell cycle disorders associated with aberrant caspase-3 regulation and/or function, proliferative disorders, proliferative disorders of the pancreas, human pancreatic carcinoma, proliferative disorders of the lung, nonsmall-cell lung cancer, proliferative disorders of the colon, colon cancer, proliferative disorders of the skin, skin cancer, proliferative disorders of the stomach, proliferative disorders of the gastrointestinal system, gastric cancer, MDM2-dependent proliferative disorders, checkpoint kinase 2 related disorders, G1 cell cycle checkpoint disorders, G2 cell cycle checkpoint disorders, aberrant cell cycle checkpoint protein disorders, disorders associated with aberrant CDK2 protein expression and/or activity, proliferative disorders of the immune system, proliferative disorders of leukemic cells, malignant lymphoma, proliferative disorders of the ovary, epithelial ovarian tumors, neural disorders, neurodegenerative disorders, Alzheimers, disorders associated with aberrant amyloid-beta expression and/or activity, disorders associated with aberrant NFKB expression and/or activity, disorders associated with aberrant cytokine expression and/or activity, disorders associated with high levels of oxidant-free radicals, disorders associated with high levels of ultraviolet irradiation, inflammatory disorders, rheumatoid arthritis, aberrant immune cell development, aberrant immune cell growth, disorders associated with aberrant vascular endothelial growth factor C expression and/or activity, disorders associated with tumor lymphangiogenesis, tumor metastasis process, proliferative disorder of the breast, breast cancer, disorders associated with aberrant heregulin-beta 1 expression and/or activity, disorders associated with interleukin-1 beta activity and/or expression, disorders associated with aberrant angiogenic potential of tissues, tumors, pancreatic adenocarcinoma, disorders associated with aberrant neutrophil migration, bone disorders, disorders associated with aberrant osteoblast differentiation, proliferative disorders of bone cells and tissues, osteosarcomas, disorders associated with aberrant expression and/or activity of bone morphogenic proteins (BMP) 4, disorders associated with aberrant expression and/or activity of BMP7, disorders associated with aberrant expression and/or activity of Cbfa1, disorders associated with aberrant osteoblast differentiation, autoimmune disorders, arthritis, asthma, septic shock, lung fibrosis, glomerulonephritis, atherosclerosis, AIDS, aberrant apoptosis, inappropriate immune cell development, delayed cell growth, disorders associated with aberrant expression and/or activity of cAMP-response element binding protein (CREB1), acute myeloid leukemia, reproductive disorders, spermatogenesis, major depressive disorder, neuropathies, Huntington's disease, disorders associated with aberrant N-cadherin expression and/or activity, disorders associated with aberrant G-protein coupled receptor regulation and/or expression, pain disorders, chronic pain, restinosis, restinosis of vascular smooth muscle cells, disorders associated with neointima formation, proliferative lesions, and proliferative lesions in mammalian blood vessels.

11. A method of treating restenosis in a host, wherein said method comprises the step of introducing an RNAi reagent selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12; into vascular smooth muscle cells at the site of a vascular lesion, wherein said cells are capable of resulting in restenosis as a result of neointima formation, in an effect amount to inhibit said neointima formation.

12. A method of inhibiting proliferative lesion formation in a blood vessel, said method comprising the step of introducing into vascular smooth muscle cells of said blood vessel an RNAi reagent selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11; in an amount sufficient to inhibit proliferative lesion formation in said blood vessel.

13. The method according to a member of the group consisting of: claim 11, and claim 12; wherein said RNAi reagent is a member of group consisting of: single stranded; and double stranded.

14. The method according to a member of the group consisting of: claim 11, and claim 12; wherein said RNAi reagent is comprised of a member of the group consisting of: deoxyribonucleotides; ribonucleotides; both deoxyribonucleotides and ribonucleotides; DNA; RNA; and RNA/DNA chimera.

15. The method according to claim 11, wherein said host is a member of the group consisting of: mammalian host, and human host.

16. The method according to claim 11 wherein said step is performed according to a condition selected from a member of the group consisting of: in vitro, in vivo, and ex vivo.

17. The method according to claim 12, wherein said blood vessel is a member of the group consisting of: mammalian blood vessel, and human blood vessel.

18. The method according to claim 12 wherein said step is performed according to a condition selected from a member of the group consisting of: in vitro, in vivo, and ex vivo.

19. A method of identifying a compound that modulates the biological activity of a polypeptide member of the group consisting of: E2F1, NFkB, CDC2A, and CREB-1, or a biological pathway of said polypeptide member, or a downstream effector of said polypeptide member; comprising the steps of, (a) combining a candidate modulator compound with said polypeptide member in the presence of a nucleic acid that antagonizes the expression and/or activity of said polypeptide member wherein said nucleic acid is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, and SEQ ID NO:35, and (b) identifying candidate compounds that reverse the antagonizing effect of said nucleic acid member.

20. The method according to claim 9, wherein said method comprises the administration of at least one said RNAi reagent, any combination of said RNAi reagents thereof, or combination of said RNAi reagent or reagents with a modulator of one or more transcription factors.


SEQUENCE LISTING (40 sequences)

SEQ ID NO: 1 - DNA, 21 bp, Homo sapiens
aaacaaggcccgatcgatgtt

SEQ ID NO: 2 - DNA, 21 bp, Homo sapiens
aagtcacgctatgagacctca

SEQ ID NO: 3 - DNA, 21 bp, Homo sapiens
aagcggaggctggacctggaa

SEQ ID NO: 4 - DNA, 21 bp, Homo sapiens
aagccgtggactcttcggaga

SEQ ID NO: 5 - DNA, 21 bp, Artificial Sequence
  Synthesized Oligonucleotide.
acaaggcccgaucgauguutt

SEQ ID NO: 6 - DNA, 21 bp, Artificial Sequence
  Synthesized Oligonucleotide.
gucacgcuaugagaccucatt

SEQ ID NO: 7 - DNA, 21 bp, Artificial Sequence
  Synthesized Oligonucleotide.
gcggaggcuggaccuggaatt

SEQ ID NO: 8 - DNA, 21 bp, Artificial Sequence
  Synthesized Oligonucleotide.
gccguggacucuucggagatt

SEQ ID NO: 9 - DNA, 21 bp, Artificial Sequence
  Synthesized Oligonucleotide.
aacaucgaucgggccuugutt

SEQ ID NO: 10 - DNA, 21 bp, Artificial Sequence
  Synthesized Oligonucleotide.
ugaggucucauagcgugactt

SEQ ID NO: 11 - DNA, 21 bp, Artificial Sequence
  Synthesized Oligonucleotide.
uuccagguccagccuccgctt

SEQ ID NO: 12 - DNA, 21 bp, Artificial Sequence
  Synthesized Oligonucleotide.
ucuccgaagaguccacggctt

SEQ ID NO: 13 - DNA, 21 bp, Artificial Sequence
  Synthesized Oligonucleotide.
gaucaauggcuacacaggatt

SEQ ID NO: 14 - DNA, 21 bp, Artificial Sequence
  Synthesized Oligonucleotide.
ccugggaauccagugugugtt

SEQ ID NO: 15 - DNA, 21 bp, Artificial Sequence
  Synthesized Oligonucleotide.
gagcaucaugaagaagagutt

SEQ ID NO: 16 - DNA, 21 bp, Artificial Sequence
  Synthesized Oligonucleotide.
cagcacagacccagcugugtt

SEQ ID NO: 17 - DNA, 21 bp, Artificial Sequence
  Synthesized Oligonucleotide.
uccuguguagccauugauctt

SEQ ID NO: 18 - DNA, 21 bp, Artificial Sequence
  Synthesized Oligonucleotide.
cacacacuggauucccaggtt

SEQ ID NO: 19 - DNA, 21 bp, Artificial Sequence
  Synthesized Oligonucleotide.
acucuucuucaugaugcuctt

SEQ ID NO: 20 - DNA, 21 bp, Artificial Sequence
  Synthesized Oligonucleotide.
cacagcugggucugugcugtt

SEQ ID NO: 21 - DNA, 21 bp, Homo sapiens
aagcccagccacagauugcca

SEQ ID NO: 22 - DNA, 21 bp, Homo sapiens
aaugggcagacaguucaaguc

SEQ ID NO: 23 - DNA, 21 bp, Homo sapiens
aacugauucccaaaagcgaag

SEQ ID NO: 24 - DNA, 21 bp, Homo sapiens
aaccaaguuguuguucaagcu

SEQ ID NO: 25 - DNA, 21 bp, Artificial Sequence
  Synthesized Oligonucleotide.
gcccagccacagauugccatt

SEQ ID NO: 26 - DNA, 21 bp, Artificial Sequence
  Synthesized Oligonucleotide.
ugggcagacaguucaaguctt

SEQ ID NO: 27 - DNA, 21 bp, Artificial Sequence
  Synthesized Oligonucleotide.
cugauucccaaaagcgaagtt

SEQ ID NO: 28 - DNA, 21 bp, Artificial Sequence
  Synthesized Oligonucleotide.
ccaaguuguuguucaagcutt

SEQ ID NO: 29 - DNA, 21 bp, Artificial Sequence
  Synthesized Oligonucleotide.
uggcaaucuguggcugggctt

SEQ ID NO: 30 - DNA, 21 bp, Artificial Sequence
  Synthesized Oligonucleotide.
gacuugaacugucugcccatt

SEQ ID NO: 31 - DNA, 21 bp, Artificial Sequence
  Synthesized Oligonucleotide.
cuucgcuuuugggaaucagtt

SEQ ID NO: 32 - DNA, 21 bp, Artificial Sequence
  Synthesized Oligonucleotide.
agcuugaacaacaacuuggtt

SEQ ID NO: 33 - DNA, 21 bp, Homo sapiens
aaggggttcctagtactgcaa

SEQ ID NO: 34 - DNA, 21 bp, Artificial Sequence
  Synthesized Oligonucleotide.
gggguuccuaguacugcaatt

SEQ ID NO: 35 - DNA, 21 bp, Artificial Sequence
  Synthesized Oligonucleotide.
uugcaguacuaggaacccctt

SEQ ID NO: 36 - DNA, 25 bp, Homo sapiens
tccagtggctgggcagccacaccac

SEQ ID NO: 37 - DNA, 25 bp, Homo sapiens
aataagccgggatctaccataccca

SEQ ID NO: 38 - DNA, 25 bp, Homo sapiens
ctgcactgccaagactgagtggttg

SEQ ID NO: 39 - DNA, 25 bp, Homo sapiens
ctgcactgccaagactgagtggttg

SEQ ID NO: 40 - DNA, 25 bp, Homo sapiens
ccgaggagctgctgcaaatggagct